(19) United States
(12) Patent Application Publication
(10) Pub. No.: US 2025/0090695 A1
(43) Pub. Date: Mar. 20, 2025

(54) IMMUNOSTIMULANT-CYTOTOXIC CONJUGATE COMPOSITION AND METHODS FOR CANCER TREATMENT

(71) Applicant: The Research Foundation for the State University of New York, Albany, NY (US)

(72) Inventors: Labros MEIMETIS, Stony Brook, NY (US); Eszter BOROS, Stony Brook, NY (US)

(21) Appl. No.: 18/828,267

(22) Filed: Sep. 9, 2024

Related U.S. Application Data
(63) Continuation of application No. PCT/US2023/064121, filed on Mar. 10, 2023.
(60) Provisional application No. 63/318,439, filed on Mar. 10, 2022.

(51) Int. Cl. A61K 51/04; A61K 51/08; A61P 35/00

(52) U.S. Cl. CPC A61K 51/0455; A61K 51/0497; A61K 51/088; A61P 35/00

(57) ABSTRACT

Provided herein are compounds and compositions for the treatment of diseases or conditions such as cancer. Also provided herein methods of treating diseases and conditions and methods of making compounds and compositions.

### CROSS REFERENCE TO RELATED APPLICATION

This application is a continuation of PCT/US2023/064121, filed Mar. 10, 2023, which claims the benefit of U.S. Provisional Application No. 63/318,439, filed Mar. 10, 2022, the disclosures of which are incorporated herein by reference in their entirety.

### INCORPORATION BY REFERENCE

All publications, patents, and patent applications mentioned in this specification are herein to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Prostate cancer is one of the leading causes of cancer death, with 1 in 10 men diagnosed in their lifetime and 1 in 41 dying from the disease. When diagnosed early, the five-year survival remains high. Unfortunately, the disease often progresses to castrate resistant prostate cancer (CRPC) and if left untreated, will progress to the metastatic castration resistant prostate cancer (mCRPC) in up to 50% of cases for which there is no cure. The prostate-specific membrane antigen (PSMA) is an attractive, well-validated target for this indication as it is highly expressed in poorly differentiated, metastatic and hormone-refractory adenocarcinomas.

Prostate cancer is one of the leading causes of cancer death, with 1 in 10 men diagnosed in their lifetime and 1 in 41 dying from the disease. When diagnosed early there is a good survival rate, unfortunately, the disease often progresses to castrate resistant prostate cancer (CRPC) and left untreated will progress to a metastatic form (mCRPC) in up to 50% of cases for which there is no cure. The prostate-specific membrane antigen (PSMA) is an attractive target for this indication as it is highly expressed in poorly differentiated, metastatic and hormone-refractory-refractory adenocarcinomas. This has been leveraged in clinical trials using the radiotherapeutic 117Lu-PSMA-617. Unfortunately, 30% of these patients are non-responders, with the major contributors being tumor heterogeneity and low PSMA expression. Additionally, in patients that do respond, the disease will eventually re-emerge and be refractory to all current treatments. There is thus a need to develop novel treatment paradigms. One approach would be to combine the cytotoxic benefits of a radionuclide with an immunostimulant to illicit a long term adaptive immune response. This could improve the likelihood of rescuing the non-responder patient population and provide enhanced clinical outcomes in the remaining patient population.

Globally there are 1.3 million new cases of prostate cancer every year and 360,000 associated deaths. Up to 50% of patients will eventually become refractory to androgen deprivation therapy and progress to castrate resistant prostate cancer (CRPC) and its metastatic form (mCRPC), eventually succumbing to the disease or die from complications. We believe that our approach will benefit multiple stake holders: 1) Patients will have better clinical outcomes, which will result in reduced suffering, 2) Clinicians will have a unique therapeutic to utilize in their armamentarium and 3) The payer (insurance companies) will benefit from limited rounds of therapy and longer symptom free survival, thus de-burdening the disease associated costs. The evidence to support our proposed product/solution is that externally administered localized radiation therapy in combination with an intratumoral injection of an immunostimulant has been shown to have a profound effect on tumor regression in patients. (4) Unfortunately, this therapy is limited by tumor accessibility and has limited clinical use. A solution is to deliver an immunostimulant and radiation to all tumor sites. A proposed therapeutic described herein is a novel small molecule drug conjugate that incorporates: 1) A PSMA targeting motif, 2) The cytotoxic radiometal ¹⁷⁷Lu already employed in FDA-approved targeted radiotherapeutic applications (t112=6.65d, W=497 keV (78.6%), 384 keV (9.1%) and 176 keV (12.2%) and 3). A small molecule immunostimulant that is released in the tumor microenvironment.

Specifically, an approach described herein incorporates a small molecule agonist of toll-like receptors (TLRs) which are important pattern recognition receptors of the innate immune system.

To address this unmet need, several clinical candidates have been approved or are under clinical investigation including: 1) Small molecule cytotoxins such as cabazitaxel, enzalutamide, apalutamide, and darolutamide, 2) Radiotherapy with Radium-223 to treat patients with mCRPC with extensive bone metastasis, 3) ¹⁷⁷Lu-PSMA-617, a prostate-specific membrane antigen (PSMA) targeting agent that incorporates the cytotoxic radiometal ¹⁷⁷Lu (t_{1/2}=6.65 d, β⁻=497 keV), and 4) The immunotherapy cancer vaccine, Sipuleucel-T, activates a patient's own immune system to mount an adaptive immune response against the tumor associated antigen, prostatic acid phosphatase. While these recent advances in treating CRPC and mCRPC have provided improvements in patient outcomes, none provide long term remission of the disease. In the case of treating mCRPC, contemporary immuno-oncology is shifting towards leveraging immunostimulants to reap the benefits of innate and adaptive immune responses.

### BRIEF SUMMARY

The present disclosure relates to compounds, compositions, and pharmaceutical compositions for treating metastatic castration resistant prostate cancer (mCRPC), and methods thereof. Methods described herein include administering a compound described herein to a patient in need thereof. In another embodiment, the disclosure includes preparing a therapeutic radio-immunostimulant. In another embodiment, the disclosure includes methods of administering a radio-immunostimulant. One class of compounds (radio-immunostimulants) described herein includes compounds of the following formula:

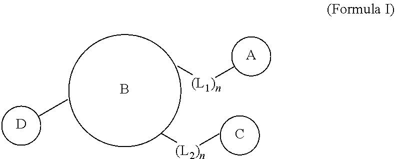

a diastereomer or enantiomer thereof, or a pharmaceutically acceptable salt of any of the foregoing, or a deuterated derivative or any of the forgoing, or a radioisotope of any of the forgoing. In some embodiments, A, L₁, B, D, L₂, and C groups are each independently covalently connected as one molecule; A comprises a: taxoid, vinca alkaloid, anthracycline, maytansinoid, tubulysin, auristatin, exatecan, duocarmycin, Seco-Cyclopropabenzindol-4-One dimer or monomer, pyrrolobenzodiazepine dimer or monomer, hemiasterlin, ²¹²Pb-DOTA, ¹⁷⁷Lu-DOTA (1,4,7,10-Tetraazacyclododecane-1,4,7,10-tetraacetic acid), ⁹⁰Y-DOTA, ²²⁵Ac-DOTA, ⁴⁷Sc-DOTA, ⁶⁷Cu-DOTA, ¹³¹I-L-Tyrosine, or any combination thereof; when A is ¹⁷⁷Lu-DOTA (1,4,7,10-Tetraazacyclododecane-1,4,7,10-tetraacetic acid), ⁹⁰Y-DOTA, ²¹²Pb-DOTA, ²²⁵Ac-DOTA, ⁴⁷Sc-DOTA, ⁶⁷Cu-DOTA, ¹³¹I-L-Tyrosine, or any combination thereof, A is covalently bound to L₁ by a C—C or C—N bond; L₁ and L₂ are each independently selected from: (CH₂)ₙ, a dipeptide, a tripeptide, a disulfide, a hydrazone, a beta glucan,

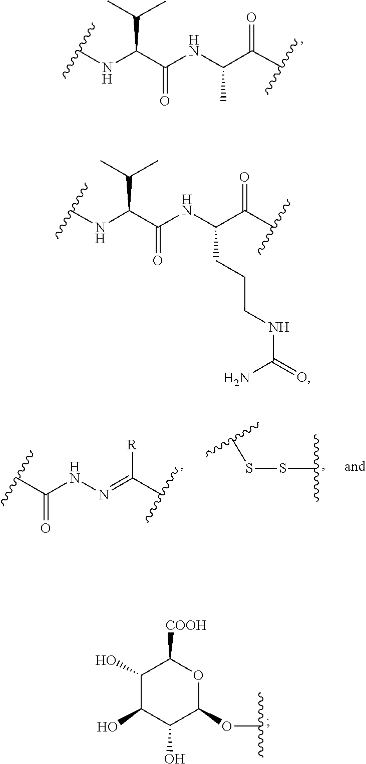

R is an aliphatic or unsaturated C1-C10, or an aromatic C6 or C10 group, wherein each R is optionally substituted with an amide, an ester, a C3-C10 cycloalkyl, an ether, or C1-C4 group; each n is independently an integer 0-8; B is selected from the group consisting of:

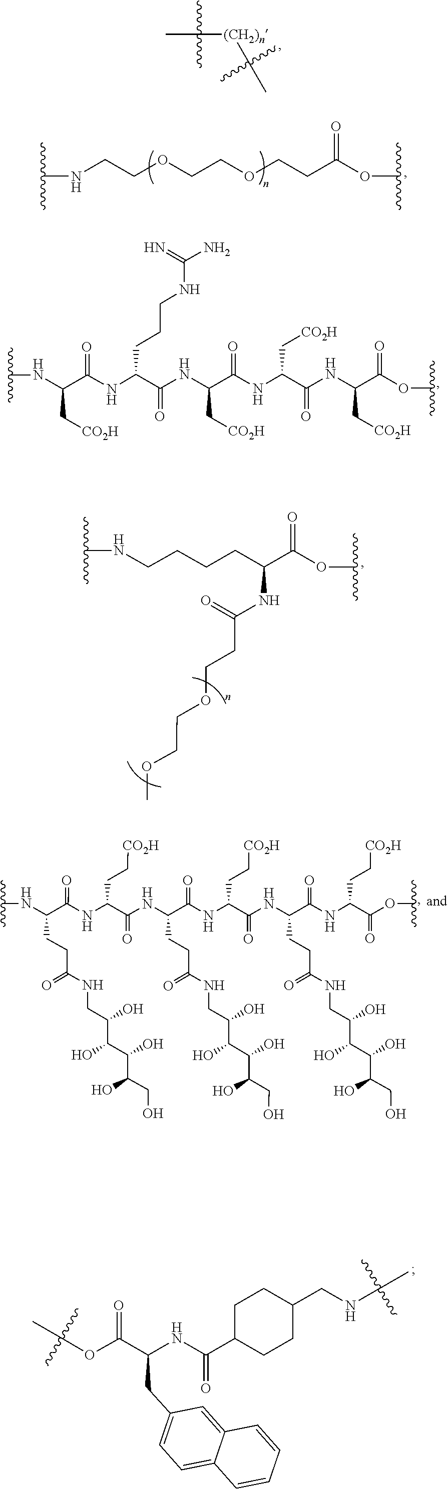

C is an immunostimulant toll-like receptor (TLR) ligand comprising a: TLR2 agonist, TLR3 agonist, TLR4 agonist, TLR5 agonist, TLR6 agonist, TLR7 agonist, TLR7/8 agonist, TLR8 agonist, TLR9 agonist, TLR10 agonist, nucleotide oligomerization domaine (NOD)-like receptor ligand, retinoic acid-inducible (RIG)-like receptor ligand, C-type lectin receptor (CLR) ligand, a cytosolic dsDNA sensor (CDS) ligand, inflammasome inducer or stimulator of interferon genes (STING) agonist,

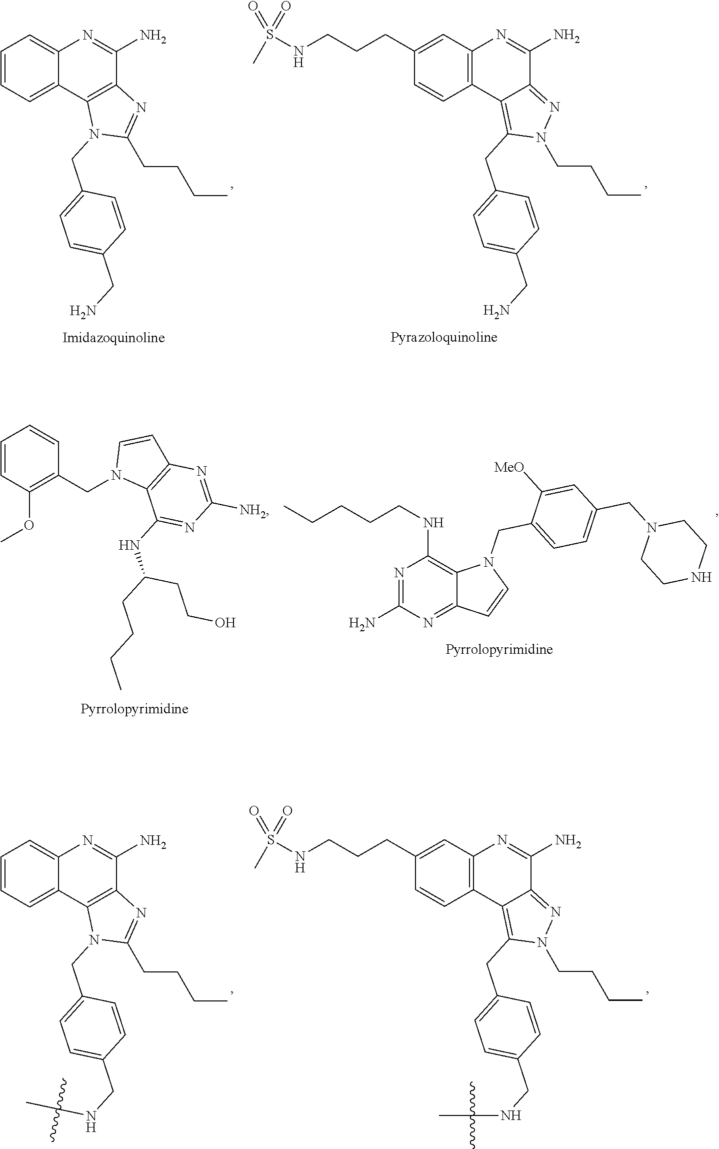

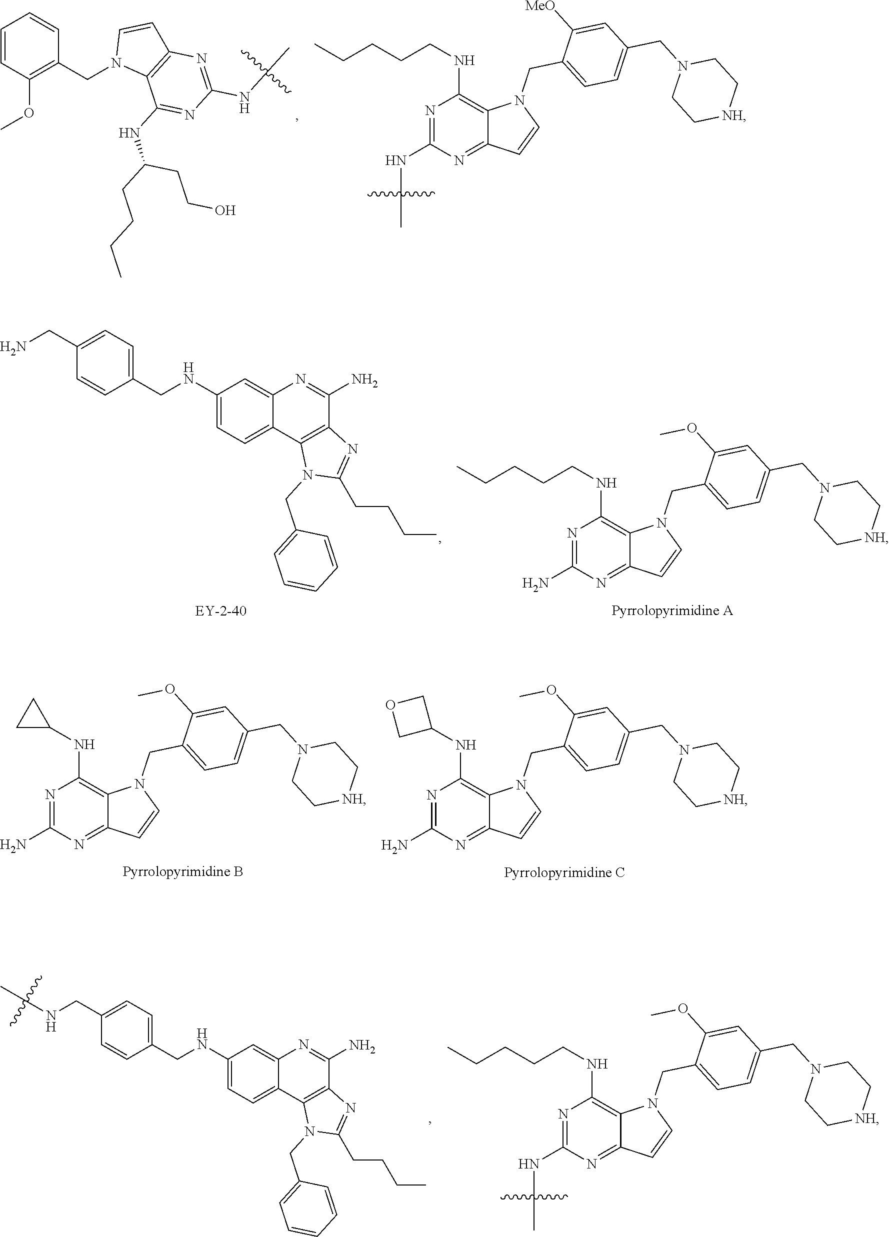

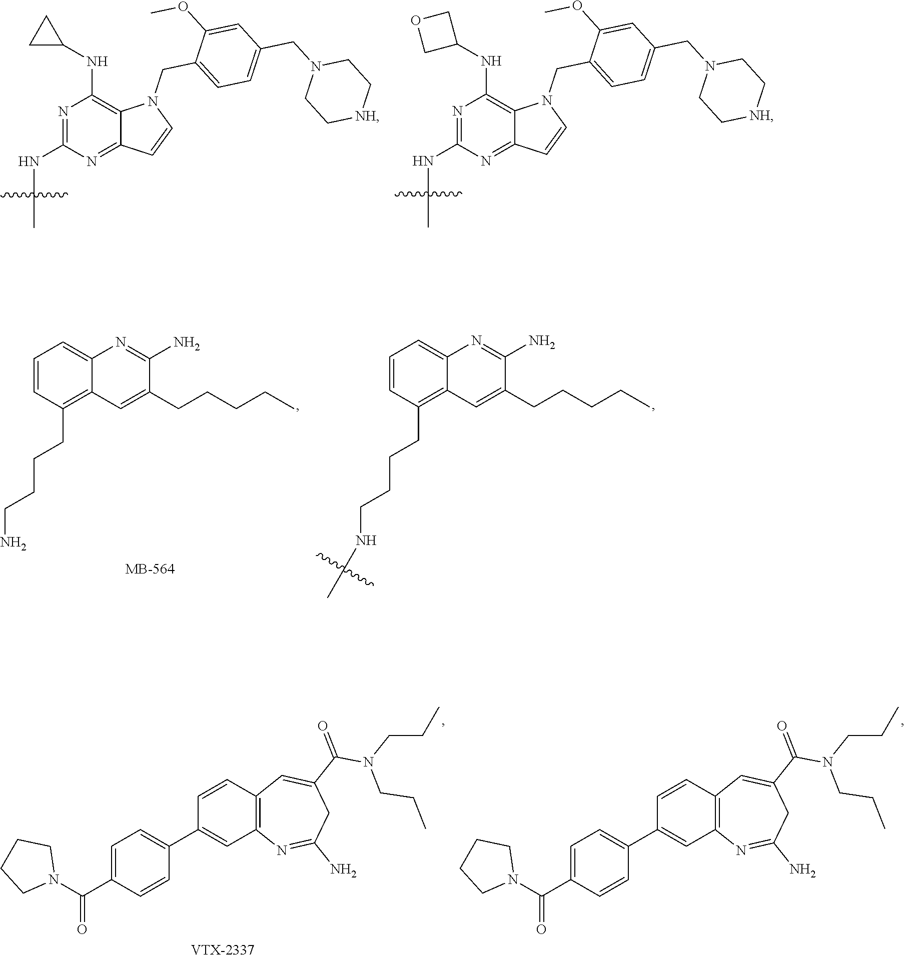

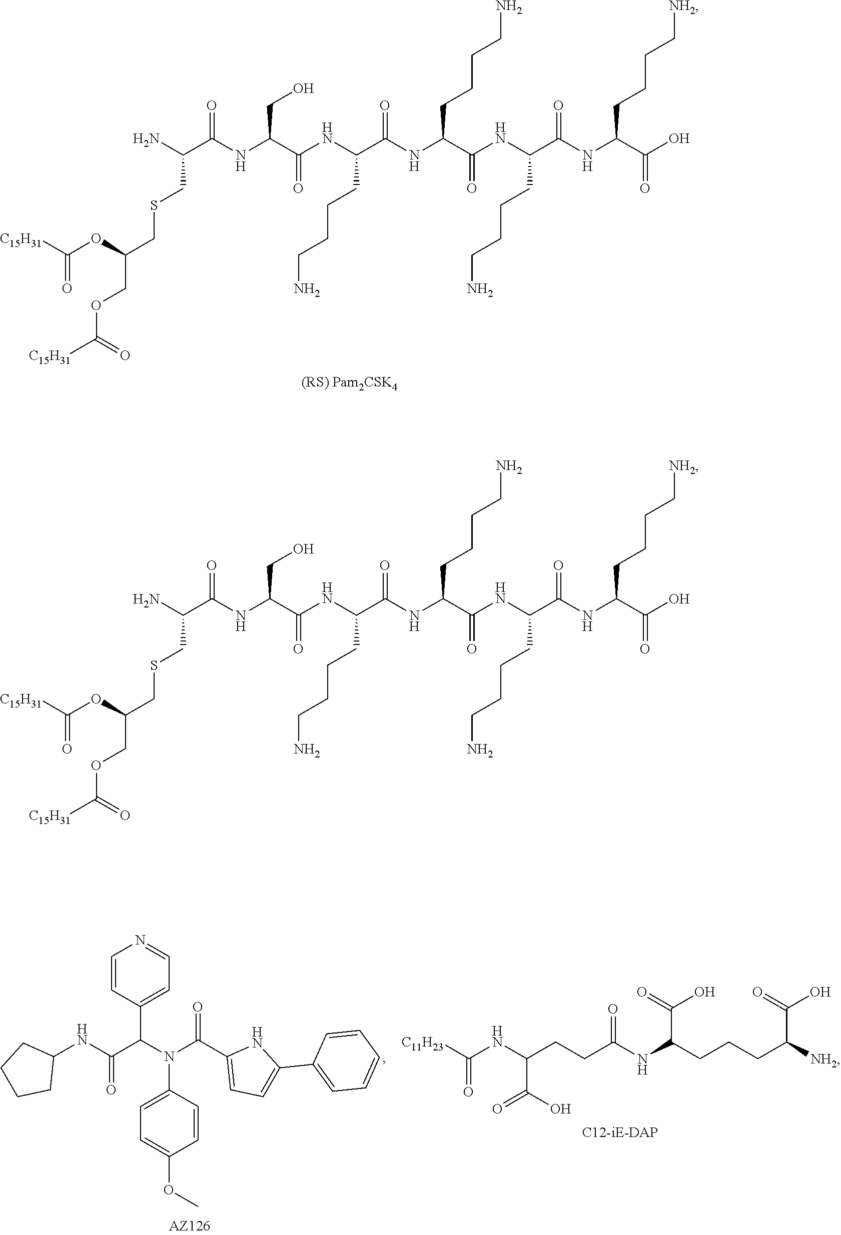

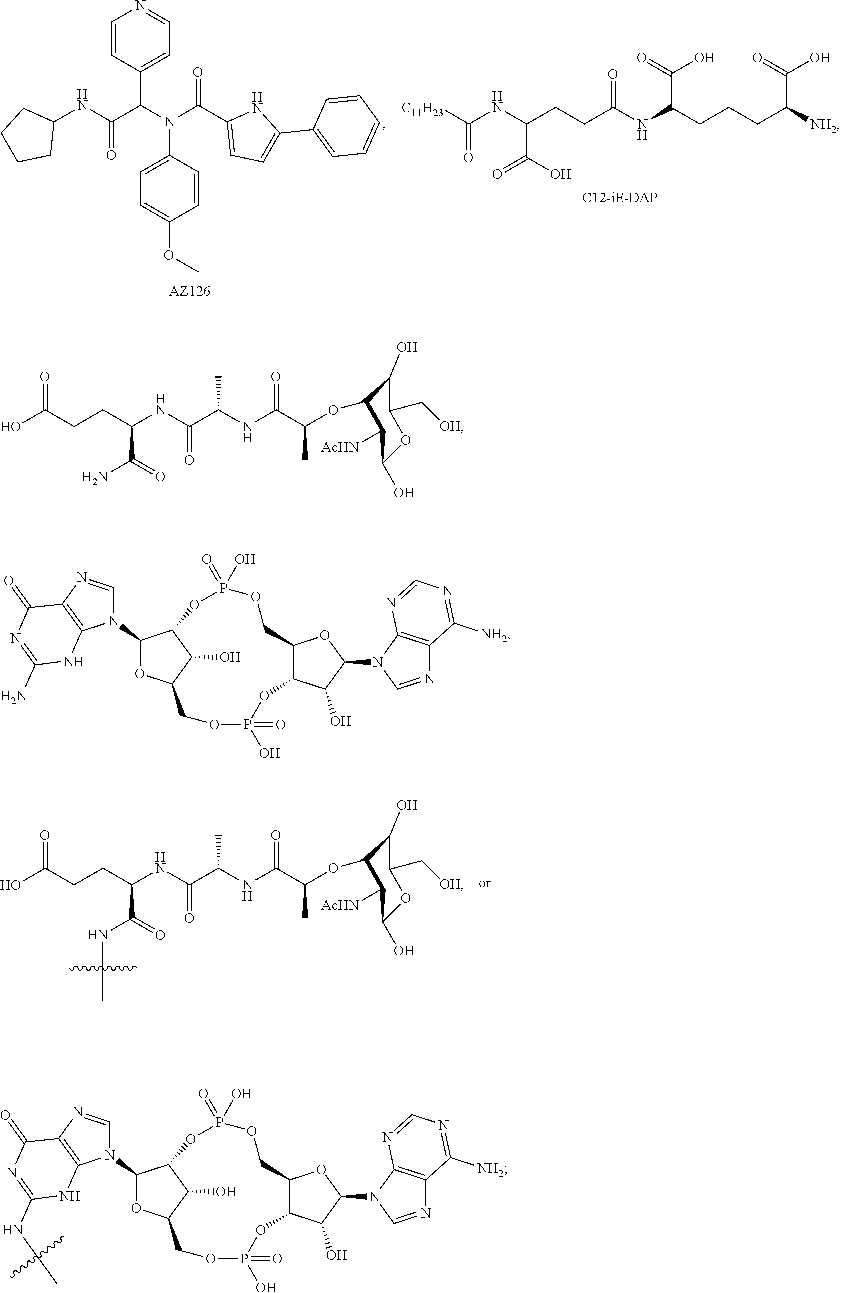

D is

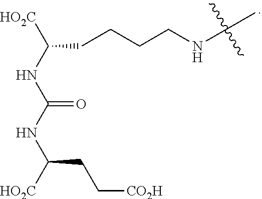 [SMILES: *NCCCC[C@H](NC(=O)N[C@@H](CCC(=O)O)C(=O)O)C(=O)O]

In some embodiments, A is selected from the group consisting of: ¹⁷⁷Lu-DOTA, ⁹⁰Y-DOTA, ²¹²Pb-DOTA, ²²⁵Ac-DOTA, ⁴⁷Sc-DOTA, ⁶⁷Cu-DOTA, and ¹³¹I-L-Tyrosine; B is:

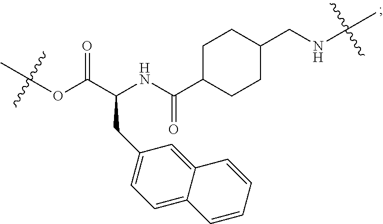 [SMILES: *NCC1CCC(C(=O)N[C@@H](Cc2ccc3ccccc3c2)C(=O)O*)CC1]

L₁ and L₂ are each independently selected from the group consisting up: (CH₂)ₙ, a dipeptide, a tripeptide, a disulfide, a hydrazone, a beta glucan,

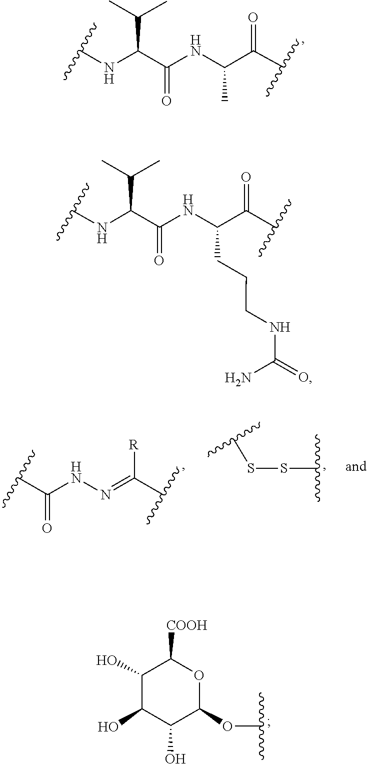

R is an aliphatic or unsaturated C1-C10, or an aromatic C6 or C10 group, wherein each R is optionally substituted with an amide, an ester, a C3-C10 cycloalkyl, an ether, or C1-C4 group;
each n is independently an integer 0-8;
C is selected from the group consisting of:

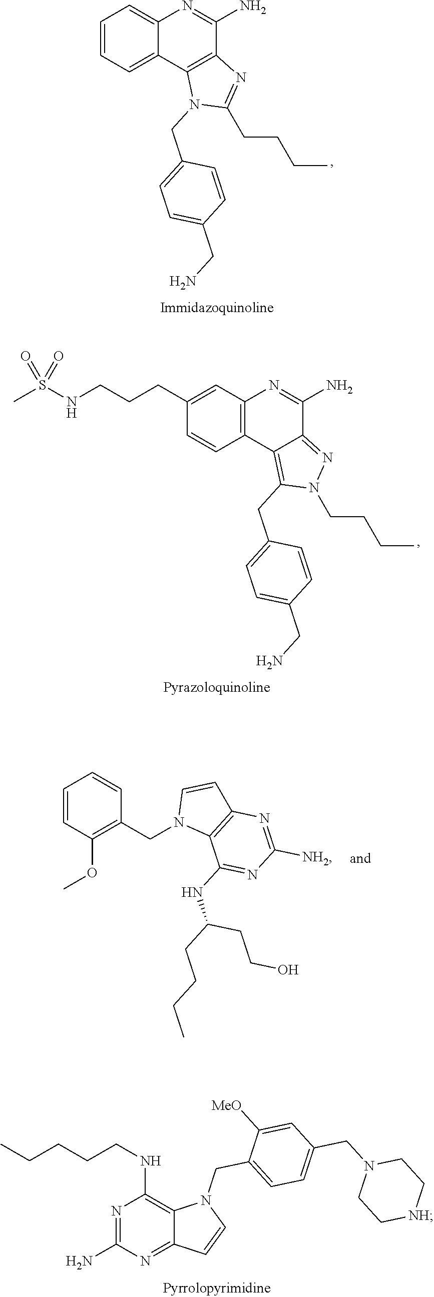

and
D is:

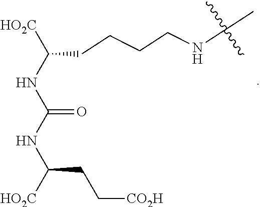 [SMILES: *NCCCC[C@H](NC(=O)N[C@@H](CCC(=O)O)C(=O)O)C(=O)O]

In some embodiments, the compound or a diastereomer or enantiomer thereof, or a pharmaceutically acceptable salt of any of the foregoing, or a deuterated derivative or any of the forgoing, or a radioisotope of any of the forgoing has the structure of Formula I, wherein:

A is ¹⁷⁷Lu-DOTA; B is:

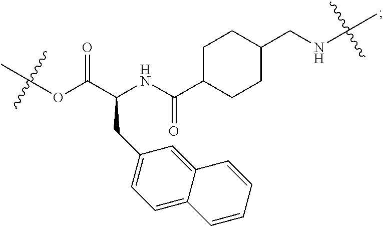 [SMILES: *NCC1CCC(C(=O)N[C@@H](Cc2ccc3ccccc3c2)C(=O)O*)CC1]

L₁ and L₂ are each independently selected from: (CH₂)ₙ, a dipeptide, a tripeptide, a disulfide, a hydrazone, a beta glucan,

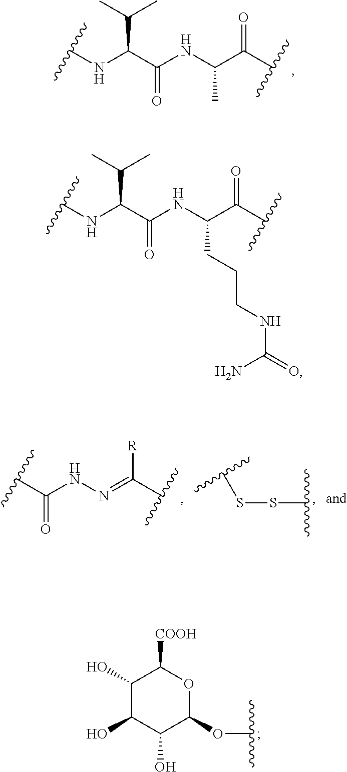

C is selected from the group consisting of:

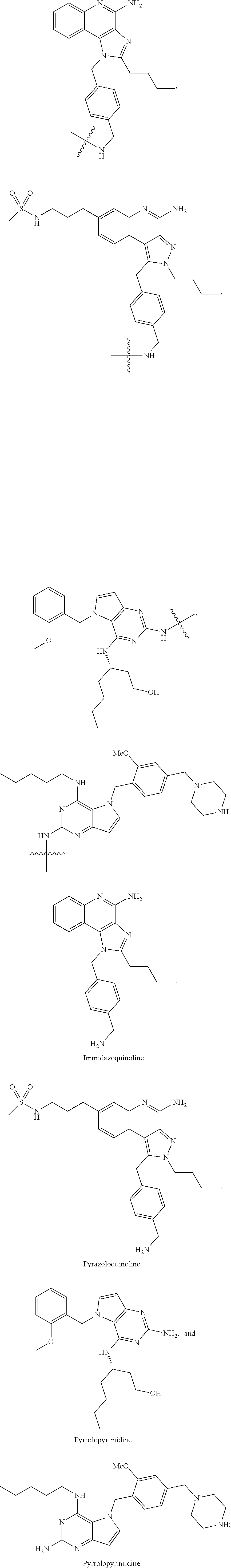

and
D is:

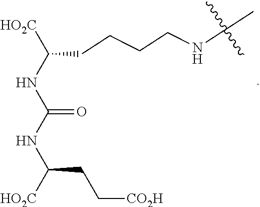 [SMILES: *NCCCC[C@H](NC(=O)N[C@@H](CCC(=O)O)C(=O)O)C(=O)O]

In some embodiments, the compound or a diastereomer or an enantiomer of the compound, or a pharmaceutically acceptable salt of any of the foregoing, a deuterated derivative of any of the foregoing, or a radioisotope of any of the forgoing, is a compound selected from the group consisting of:

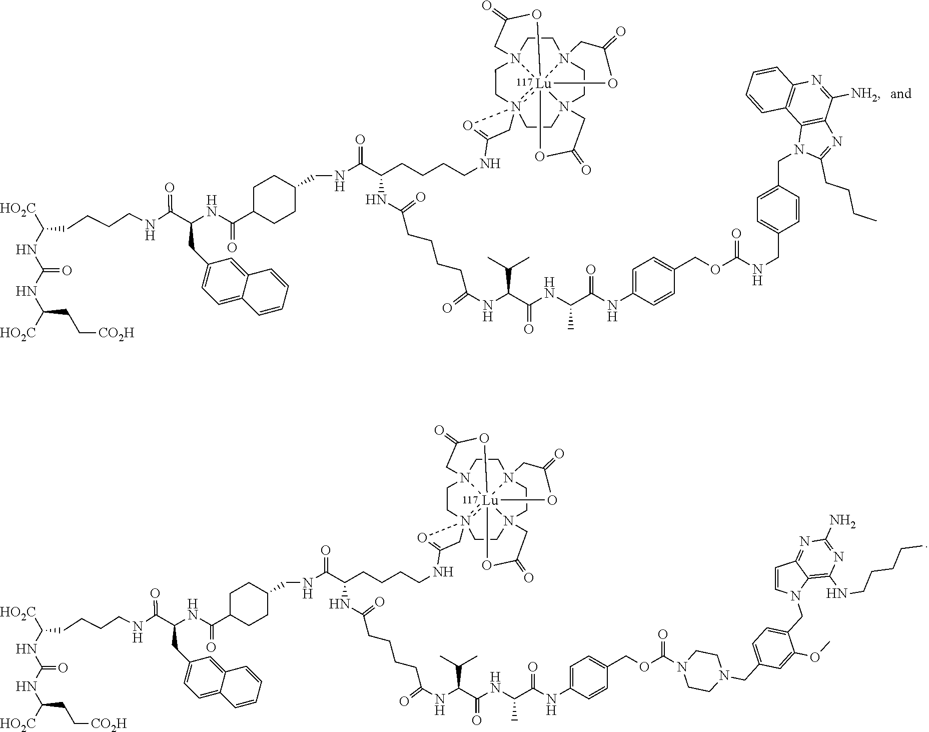

Also provided herein are compositions comprising: i) the compound of Formula I, the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing; and ii) an excipient, diluent, or carrier. In some embodiments, the composition is a pharmaceutical composition. In some embodiments, the composition comprises an additional active agent, or a pharmaceutically acceptable salt thereof. In some embodiments, the pharmaceutical composition comprises a radio-immunostimulant. In some embodiments, the pharmaceutical composition is a radio-immunostimulant. Also provided herein are kits comprising the pharmaceutical composition described herein and a container.

Also provided herein is a method of treating a cancer in a subject, the method comprising administering a pharmaceutical composition comprising a compound of Formula I, the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing, to the subject. In one embodiment is a method of treating a cancer in a subject, the method comprising administering a pharmaceutical composition comprising a compound of Formula I, the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing, to the subject in a therapeutically effective amount, thereby treating the cancer. In some embodiments, the administering is selected from the group consisting of: oral, an injection; subcutaneous, intra-tumoral; systemic, local, intravenous, intraperitoneal, intramuscular, and any combination thereof. Also provided herein is a method of decreasing the size of a tumor, the method comprising contacting the tumor with the pharmaceutical composition comprising a compound of Formula I, the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing. Also provided herein is a method of decreasing the size of a tumor, the method comprising contacting the tumor with the pharmaceutical composition comprising a compound of Formula I, the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing, in an amount effective to decrease the size of the tumor. In some embodiments, the pharmaceutical composition comprises a radio-immunostimulant (RIMS). Also provided herein is a method of administering a tumor vaccine to a subject, the method comprising administering the pharmaceutical composition described herein. Also provided herein are methods of administering an intratumoral injection comprising an immunostimulant. Also provided herein is a method for tumor regression in patients. Also provided herein is a method for tumor regression in patients, the method comprising administering a RIMS compound. Also provided herein is a method of administering a tumor vaccine to a subject, the method comprising administering the pharmaceutical composition described herein, thereby vaccinating the subject against the tumor. Also provided herein is a method of administering a tumor vaccine to a subject, the method comprising administering the pharmaceutical composition comprising a compound of Formula I, the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing. Also provided herein is a method of administering a tumor vaccine to a subject, the method comprising administering the pharmaceutical composition comprising a compound of Formula I, the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing, thereby vaccinating the subject against the tumor. In some embodiments, the tumor is a cancerous tumor, and wherein the subject has a cancer. In some embodiments, the subject has a: prostate cancer, ovarian cancer, kidney cancer, colorectal cancer, NSCL cancer, castrate resistant prostate cancer. In some embodiments, the cancer comprises the castrate resistant prostate cancer that has progressed to metastatic castrate resistant prostate cancer. Also provided herein are methods of achieving a biodistribution of a therapeutic in a tumor of a subject, the method comprising, contacting the tumor with the pharmaceutical composition comprising a compound of Formula I, the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing. In some embodiments, the pharmaceutical composition has a radioactivity measure from about 1 MBq/nmol to about 1000 MBq/nmol. Also provided herein are methods of activating an antigen presenting cell, the method comprising contacting the antigen presenting cell with the pharmaceutical composition comprising a compound of formula I.

Also provided herein are pharmaceutical compositions comprising an immunostimulant, a cancer antigen targeting agent, a spacer molecule, a first linker, a second linker, and a cytotoxic agent, wherein:
the immunostimulant is a: TLR8 agonist, TLR7 agonist, TLR2 agonist, TLR4 agonist, NOD2 agonist, NOD1 agonist, or a STING agonist, wherein
   (i) the immunostimulant is covalently bound to the first linker, and wherein the first linker is configured to release the immunostimulant when contacted with a cathepsin B mediated enzymatic cleavage; the second linker is covalently bound to the cytotoxic agent, the spacer molecule, or a combination thereof;
the cytotoxic agent comprises a radiotherapeutic, a small molecule, or a combination thereof, and wherein
   (ii) the cytotoxic agent is covalently bound to the second linker, the spacer molecule, or a combination thereof; and wherein
   (iii) the cancer antigen targeting molecule targets a PSMA cell, and is covalently bound to the spacer molecule; and
   (iv) a pharmaceutically acceptable diluent, excipient, carrier. In some embodiments, the pharmaceutical composition is a cancer vaccine in situ.

In some embodiments, the cytotoxic agent is selected from the group consisting of a: taxoid, vinca alkaloid, anthracycline, maytansinoid, tubulysin, auristatins, ¹⁷⁷Lu-H₃mpatcn, ¹⁷⁷Lu-(picaga)-DUPA, ¹⁷⁷Lu-DOTA, ⁹⁰Y-DOTA, ²²⁵Ac-DOTA, ⁴⁷Sc-DOTA, ⁴⁷Sc-(picaga)-DUPA, ⁴⁷Sc—(H₃mpatcn) ⁶⁷Cu-DOTA, and ¹³¹I-L-Tyrosine. In some embodiments, the cytotoxic agent comprises ¹⁷⁷Lu-DOTA, ¹⁷⁷Lu-H₃mpaten, ¹⁷⁷Lu-(picaga)-DUPA. In some embodiments, the cytotoxic agent comprises ¹⁷⁷Lu-DOTA. In some embodiments, the cytotoxic agent comprises ¹⁷⁷Lu-(picaga)-DUPA. In some embodiments, the cytotoxic agent has a structure of Compound 1:

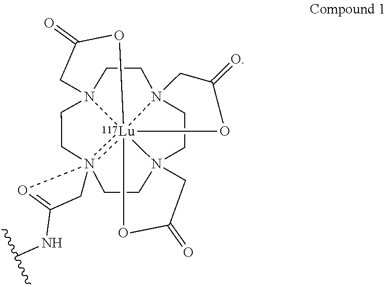

In some embodiments, the cytotoxic agent is covalently bonded to a spacer molecule by an N—C bond, and wherein the first linker connects the cytotoxic agent to the spacer by a C—C bond. In some embodiments, the spacer molecule is further covalently bonded to a cancer antigen targeting molecule, wherein the cancer antigen targeting molecule has a structure of Compound 2:

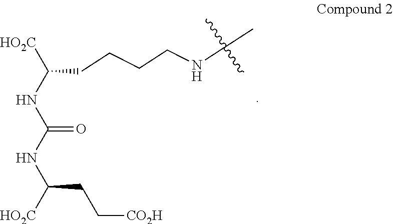 [SMILES: *NCCCC[C@H](NC(=O)N[C@@H](CCC(=O)O)C(=O)O)C(=O)O]

In some embodiments, the cancer antigen targeting molecule is covalently bonded to a spacer molecule by a N—C bond. In some embodiments, the immunostimulant is covalently bound to the second linker by a C—N or C—C bond, wherein the immunostimulant is imidazoquinoline or pyrrolopyrimidine, the second linker is covalently bound to the spacer molecule by an amide or internal amine C—N bond, and the second linker is selected from the group consisting of Compound 3,
(v)

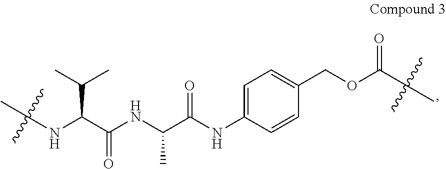 [SMILES: *N[C@H](C(=O)N[C@@H](C)C(=O)Nc1ccc(COC(*)=O)cc1)C(C)C]

a hydrazone, a disulfide, a dipeptide, beta-glucan, and a derivative or analog of any of the forgoing.

In some embodiments, the immunostimulant is covalently bound to Compound 3 or a derivative or analog thereof, and the immunostimulant is selected from the group consisting of:

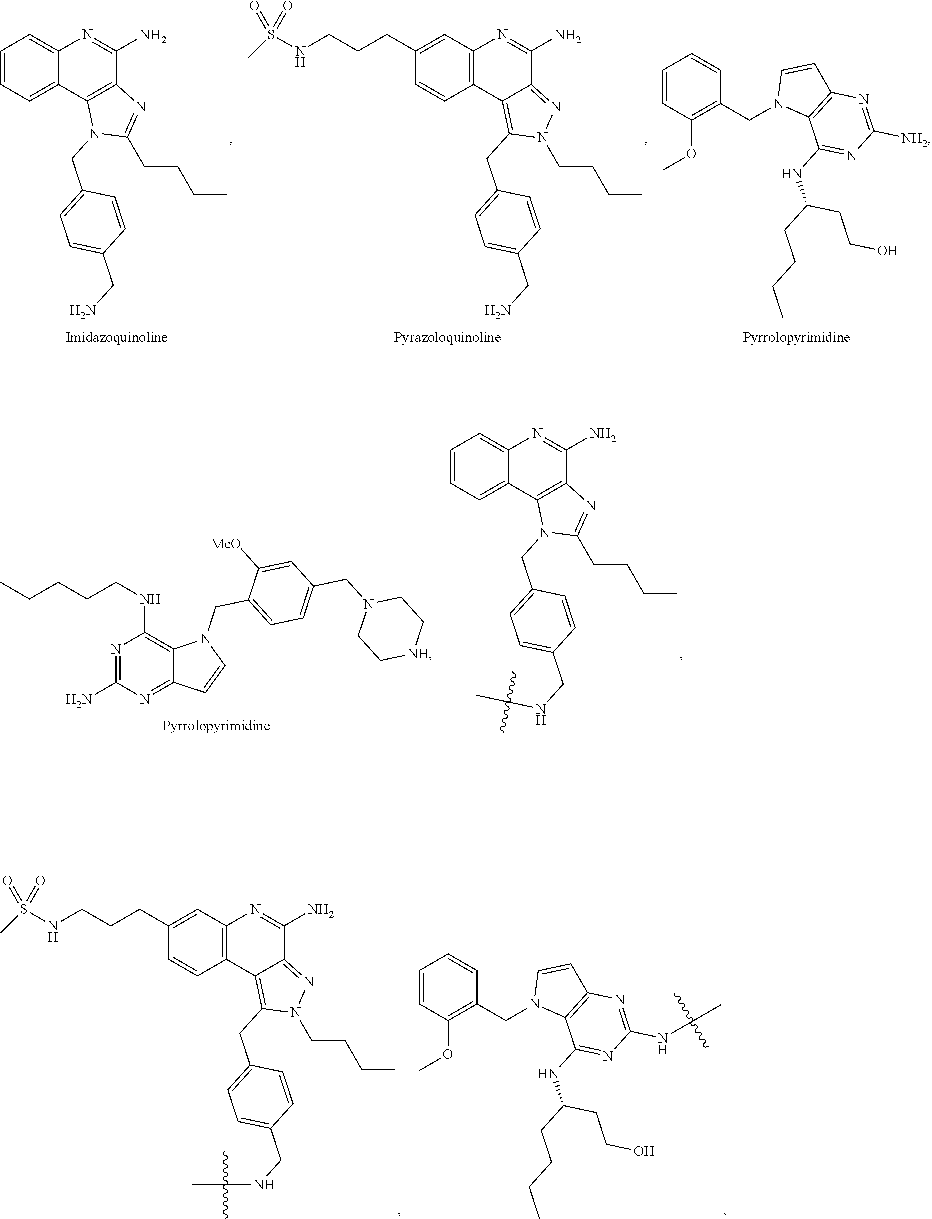

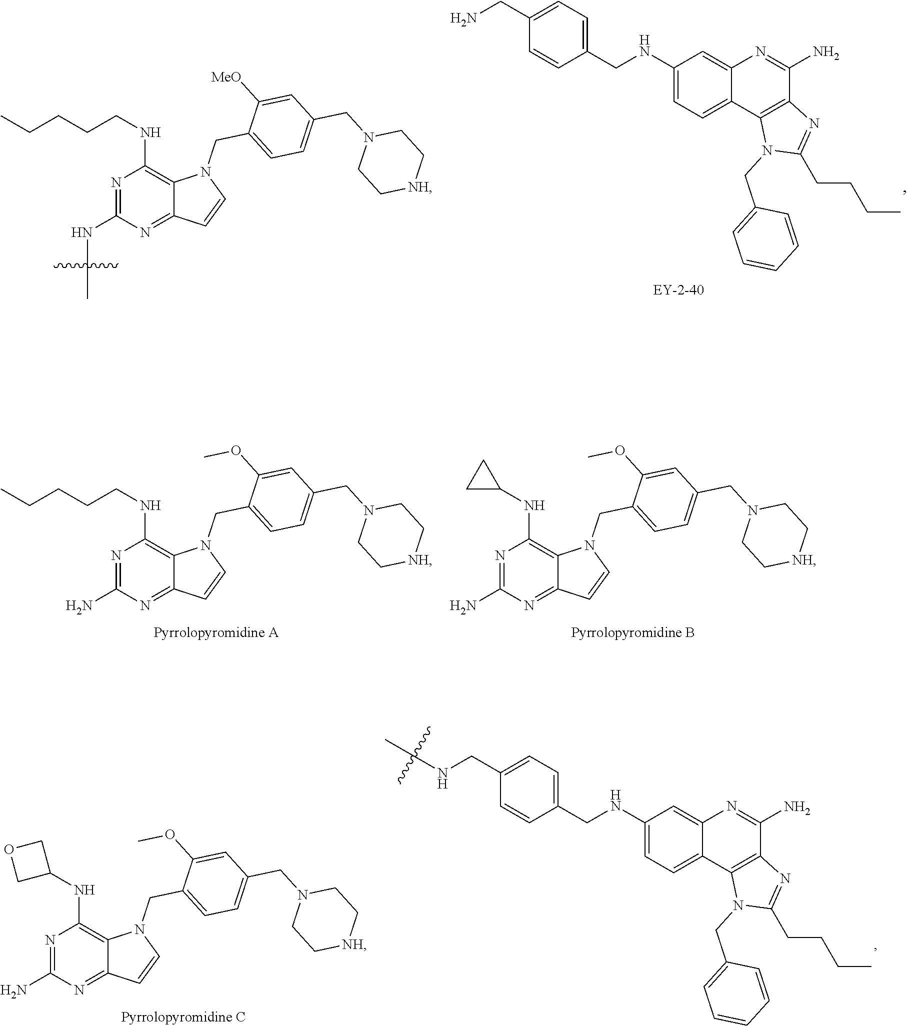 [SMILES: *Nc1nc(NCCCCC)c2c(ccn2Cc2ccc(CN3CCNCC3)cc2OC)n1 ; CCCCc1nc2c(N)nc3cc(NCc4ccc(CN)cc4)ccc3c2n1Cc1ccccc1 ; CCCCCNc1nc(N)nc2ccn(Cc3ccc(CN4CCNCC4)cc3OC)c12 ; COc1cc(CN2CCNCC2)ccc1Cn1ccc2nc(N)nc(NC3CC3)c21 ; COc1cc(CN2CCNCC2)ccc1Cn1ccc2nc(N)nc(NC3COC3)c21 ; *NCc1ccc(CNc2ccc3c(c2)nc(N)c2nc(CCCC)n(Cc4ccccc4)c23)cc1]

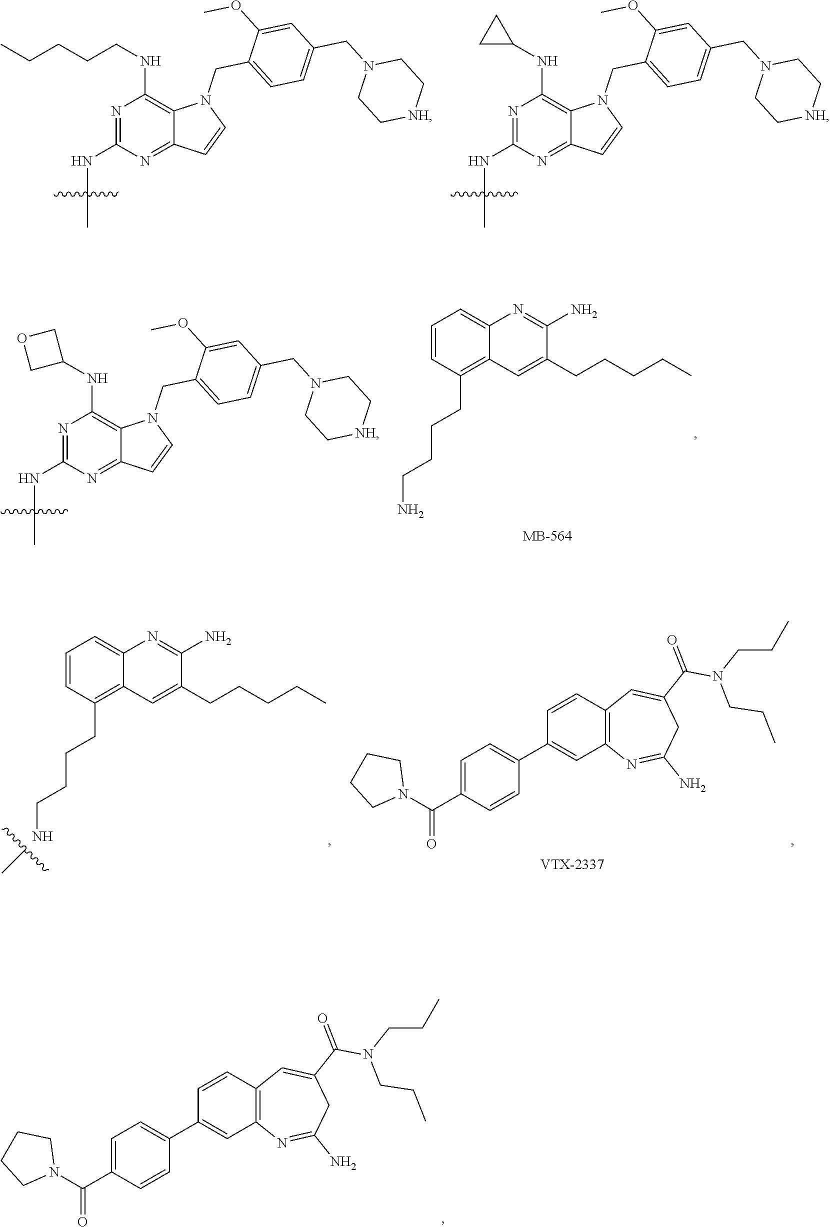

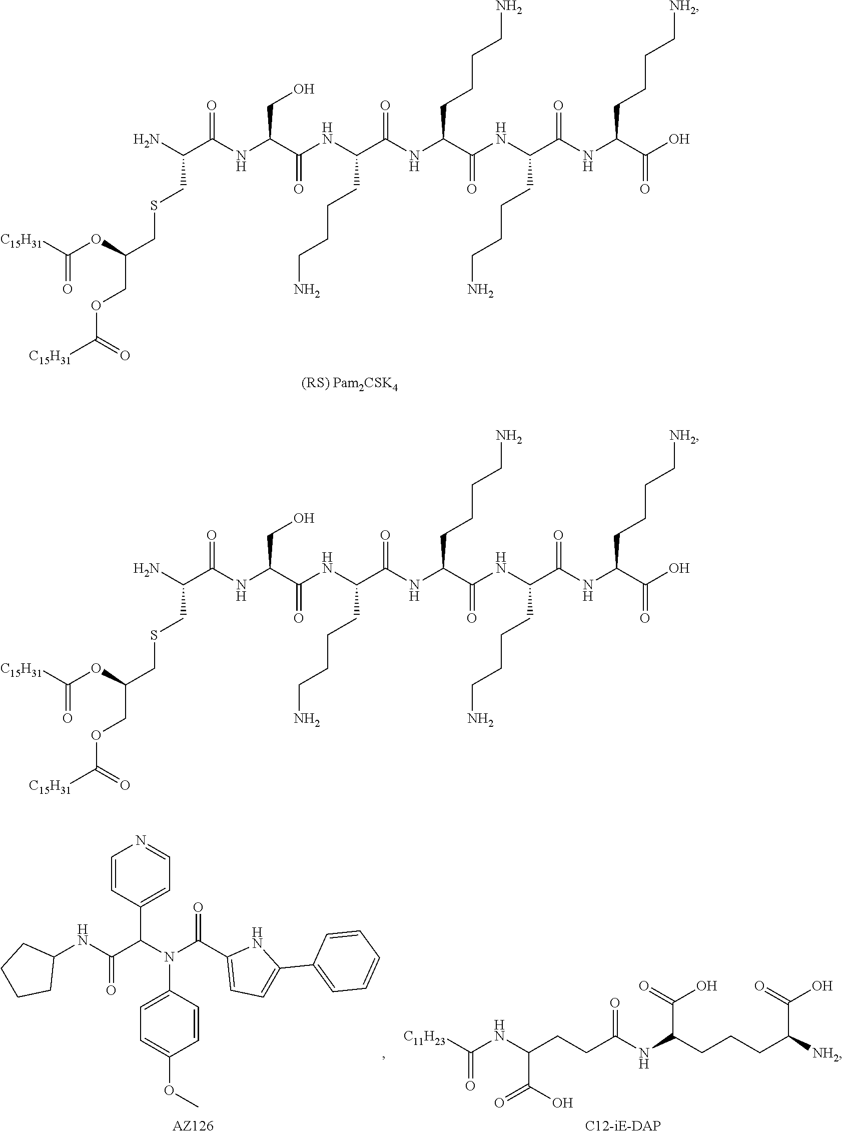

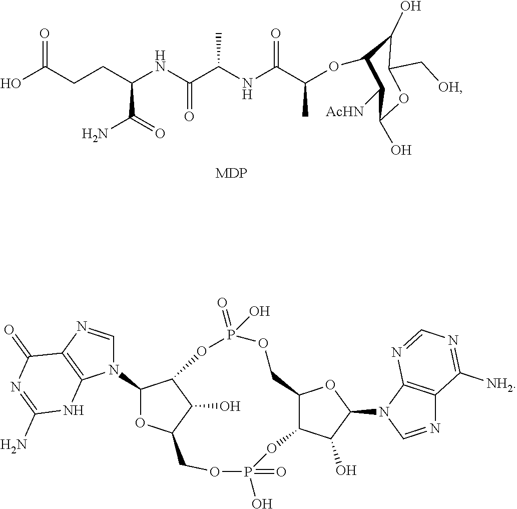

In some embodiments, the spacer molecule is selected from the group consisting of:

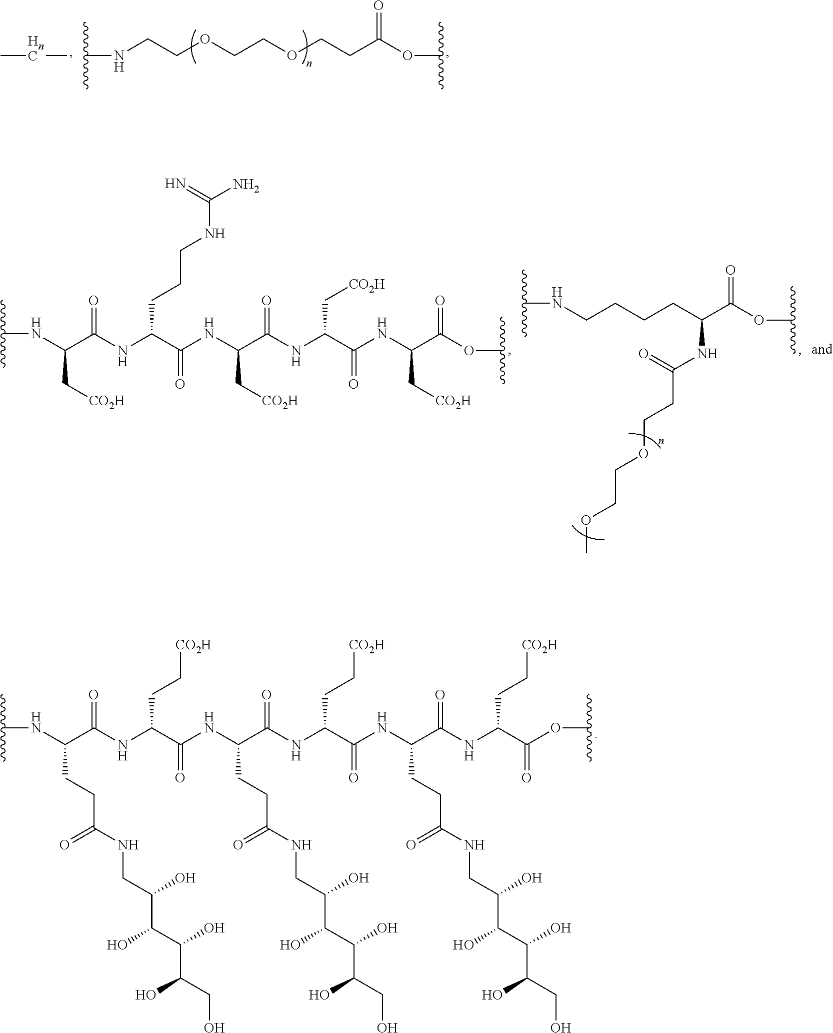

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-1C are visual schematics of a small molecule immunostimulant conjugated to a cytotoxin being delivered to a cancer cell. FIG. 1A is a visual schematic showing systemic delivery of a cytotoxin in comparison to a localized delivery approach of a cytotoxin in tandem with immune response activation. FIG. 1B shows a representation of an exemplary ¹⁷⁷Lu RIMS compound. FIG. 1C shows a representation of an exemplar RIMS compound undergoing endocytosis followed by an acid-sensitive cleavage/release of the RIMS compound's immunostimulant moiety.

FIG. 2A-2B are visual schematics. FIG. 2A illustrates the localization/activation of immune cells and increased phagocytic activity catalyzed by the TLR 7/8 agonist combined with the apoptotic cancer cell debris generated by the cytotoxic payload, results in tumor antigen loaded APC's (e.g., dendritic cells). This generates recruitment of tumor specific cytotoxic T-cells that kills cancer cells expressing the tumor associated antigens. FIG. 2B illustrates toll-like receptor agonist effects on different types of cells including monocytes/macrophages, dendritic cells, NK/NKT cells, T cells, B cells, mast cells, and tumor cells.

FIG. 3A-3B are visual schematics. FIG. 3A is a visual schematic showing (i) a treatment path for a patient's refractory to hormone therapy; and (ii) highlighting RIMS therapy. FIG. 3B is a visual schematic of the mode of action of a tumor vaccine comprising a radio-immunostimulant wherein 1) PSMA(+) PSMA(−) cells are targeted; 2) neoantigens are produced via dendritic cell phagocytosis; 3) T-cell amplification occurs; 4) and 5) represent the death of the cancer cells; and 6) depicts slowed and/or halted metastasis of PSMA(+) and PSMA(−) cancer cells.

FIG. 4 is a graphical representation of a high-performance liquid chromatography chromatogram for the analysis of an exemplary immunostimulant compound compared with its radiolabeled counterpart (¹⁷⁷Lu-complex).

FIG. 5 is a graphical representation of a RIMS displacement assay to analyze inhibition potency by calculating the inhibition constant (Kᵢ).

FIG. 6 is a graphical representation of chromatograms of an exemplary radioimmunostimulant (RIMS) undergoing enzymatic cleavage of the covalently bound cathepsin B moiety of the RIMS.

FIG. 7 is a graphical representation of metabolite detection (m1 molecular structure shown in FIG. 8) measured in in vivo dose per gram of tissue (% ID/g) in different areas of murine subjects contacted with an exemplary ¹⁷⁷Lu-RIMS compound.

FIG. 8 is a graphical representation of metabolite detection in the urine of a murine subject 2 hours after administration of an exemplary ¹⁷⁷Lu-RIMS compound. The detectable metabolite structure “m1” is shown (right).

FIG. 9A-9B show graphical representations of metabolite detection in vivo in different areas of murine subjects contacted with an exemplary ¹⁷⁷Lu-RIMS-1 compound compared with a positive control (¹⁷⁷Lu-PSMA-617) (FIG. 9A); and ¹⁷⁷Lu-RIMS-1 detection in urine of a subject (FIG. 9B).

FIG. 10 is a graphical representation of compound internalization in RM1-PGLS tumors for ¹⁷⁷Lu-RIMS versus the control ¹⁷⁷Lu-PSMA-617 compound over time.

FIG. 11A-11C show a visual schematic of RM1-PGLS tumor insertion in mice followed by T-cell population analysis (FIG. 11A), and tumor efficacy study analysis for in vivo tumor treatment with experimental ¹⁷⁷Lu-RIMS-1 compound compared with control compounds: ¹⁷⁷Lu-PSMA-617, unlabeled Lu-RIMS, and saline (FIG. 11B) measuring tumor volume (left), and percent survival (right). FIG. 11C shows body weight post treatment for murine subjects. ¹⁷⁷Lu-RIMS-1 treated subject were further monitored after reimplantation of ¹⁷⁷Lu-RIMS-1 showing a decrease in relative body weight.

FIG. 12A-12B show graphical representations of T-cell population analysis measuring cell viability in the spleen (FIG. 12A). and in the tumor (FIG. 12B) in vivo for subjects having RM1-PGLS tumors with either an experimental ¹⁷⁷Lu-RIMS compound, or a control compound selected from: ¹⁷⁷Lu-PSMA-617, unlabeled Lu-RIMS, or a control vehicle.

FIG. 13A-13C describes a method and results for T-cell detection using immuno-positron emission tomography (PET). FIG. 13A shows a visual schematic of murine subjects with RM1-PGLS tumors treated in vivo with an experimental ¹⁷⁷Lu-RIMS compound; FIG. 13B shows a graphical representation for immune response analysis by T-cell count measuring ⁸⁹Zr-DFO-antiCD3 detection; and FIG. 13C shows imaging of the subjects assess monitor for spatial T-cell distribution and T-cell tumor infiltration over time.

FIG. 14A-14D show graphical representations of tumor infiltrating T-cell analysis after administration of a therapeutic compound or control. Infiltration was measured using immuno-positron emission tomography (PET). FIG. 14A shows a visual schematic of murine subjects with RM1-PGLS tumors treated in vivo with an experimental ¹⁷⁷Lu-RIMS compound;

FIG. 14B shows treatment with ¹⁷⁷Lu-PSMA-617, FIG. 14C shows treatment with a control vehicle, and FIG. 14D shows treatment with unlabeled Lu-RIMS.

FIG. 15 shows a radio-HPLC monitoring the stability of ¹⁷⁷Lu-RIMS-1 by analysis of radiolytic degradation and decomplexation at relevant time point concentrations for dose preparation, storage, and administration.

FIG. 16 shows verification of hPSMA expression in RM1-PGLS tumors in vivo in murine subjects. FIG. 16 shows a Radio-HPLC chromatogram of 64Cu-PSMA-617 (Rt=7.58 min) representing PET imaging results of mice injected with 64Cu-PSMA-617. FIG. 16B shows tumor PET imaging results using ⁶⁴Cu-PSMA-617 which shows functional hPSMA expression in vivo in C57BL/6J mice bearing RM1-PGLS tumor (left). PET signal is blocked with the PSMA inhibitor 2-PMPA (50 nanomole) in the right PET imaging scan.

FIG. 17A-17C show exemplary immunostimulants of RIMS.

FIG. 18 shows the total amount of radioactivity per amount of immunostimulant administered as a result of ¹⁷⁷Lu-RIMS-1 cleavage.

### DETAILED DESCRIPTION

The following detailed description of embodiments of the disclosure will be made in reference to the accompanying drawings. In describing the disclosure, explanation about related functions or constructions known in the art are omitted for the sake of clearness in understanding the concept of the disclosure to avoid obscuring the disclosure with unnecessary detail. Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

### Definitions

Throughout this disclosure, various embodiments can be presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

Unless specifically stated or obvious from context, as used herein, the term “about” is understood as within a range of normal tolerance in the art, for example within 2 standard deviations of the mean. “About” can be understood as within plus or minus: 15%, 14%, 13%, 12%, 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Where particular values are described in the application and claims, unless otherwise stated, the term “about” is implicit and in this context means within an acceptable error range for the particular value.

Unless otherwise stated, structures depicted herein are also meant to include all isomeric (e.g., enantiomeric, diastereomeric, geometric (or conformational) forms of the structure; for example, the L and D designations for each asymmetric center, the R and S configurations for each asymmetric center, (Z) and (E) carbon-carbon double bond isomers, R and S configurations for each carbon or sulfur atom center, and (Z) and (E) conformational isomers. Therefore, single stereochemical (enantiomers, diastereomers) isomers (enantiomers, diastereomers) as well as enantiomeric, diastereomeric, and geometric (or conformational) mixtures of the present compounds are within the scope of the disclosure. Each independent stereocenter, unless explicitly defined, may include, a mixture of stereoisomers, or a pure stereoisomer, thereof.

Unless otherwise stated, compounds with one or more asymmetric centers referred to herein, include enantiopure, diastereomeric, diastereopure, enantioenriched, diastereoenriched, and racemic mixtures thereof.

The term “adjacent” and its grammatical equivalents as used herein refer to right next to the object of reference. For example, the term adjacent in the context of a cell or a tissue can mean without any other cells or tissues in between.

The term “analog” and its grammatical equivalents as used herein refer to a molecule that is not identical but has analogous structural features. An analog of a drug or agent is a drug or agent that is related to a reference agent, but whose chemical structure can be different. Analogues exhibit similar activities to a reference drug or agent, but the activity can be increased or decreased or otherwise improved. An analogue form of a compound or drug can mean that the backbone core of the structure is modified or changed compared to a reference drug.

The term “cancer” and its grammatical equivalents as used herein refer to a hyperproliferation of cells whose unique trait-loss of normal controls-results in unregulated growth, lack of differentiation, local tissue invasion, and metastasis.

The term “drug resistant cancer” and its grammatical equivalents as used herein refers to a cancer that does not respond, or exhibits a decreased response to, one or more chemotherapeutic agents.

The term “effective amount” or “therapeutically effective amount” and its grammatical equivalents refers to an amount that is sufficient to achieve or at least partially achieve the desired effect.

As used herein, the term “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation, amelioration, or slowing the progression, of one or more symptoms or conditions associated with a condition, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Exemplary beneficial clinical results are described herein. The terms “treating” and “treatment” may also relate to reduction in severity and/or frequency of symptoms, elimination of symptoms and/or underlying cause, prevention of the occurrence of symptoms and/or their underlying cause, and improvement or remediation of damage.

In some embodiments, a compound or a salt thereof may comprise an enantiomerically pure form. In some examples, the compound or salt thereof disclosed herein can have an enantiomeric excess greater than about or equal to: 5%, 10%, 15%, 20%, 25%, 30%, 35%, 40%, 45%, 50%, 60%, 70%, 80%, 90%, 95%, 96%, 97%, 98%, or 99%. A compound or a salt thereof may be dosed in their enantiomerically or diasteriomerically pure form. In some cases, percent enantiomeric excess can be defined as:
% ee=(|F_{R}−F_{S}|×100) wherein FR is the mole fraction of the compound with an R stereocenter and FS is the mole fraction of the compound with an S stereocenter and the two vertical lines indicate taking the absolute value of the difference.

The diastereomer excess, or de (diastereomeric excess) value, can indicate the excess of a diastereomer in a diastereomer mixture. It can be defined as:

$$de = \frac{{m}_{1} - {m}_{2}}{{m}_{1} + {m}_{2}} \cdot 100 \%$$

with: m1 being mass of the diastereomer in excess, and m2 being mass of the diastereomer in deficit. In some examples, the compound as a diastereomer or a salt thereof or pharmaceutically acceptable salt thereof or deuterated derivative thereof can have a diasteriomeric excess greater than about or equal to: 5%, 10%, 15%, 20%, 25%, 30%, 35%, 40%, 45%, 50%, 55%, 60%, 65%, 70%, 75%, 80%, 90%, 95% or 99%. With a 1:1 mixture of two diastereomers, de=0%, with a diastereomerically pure compound de=100%.

The term “hyperproliferative cells” and its grammatical equivalents as used herein refers to cells characterized by unwanted cell proliferation, or abnormally high rate or sustained cell division, unrelated or uncoordinated with that of surrounding normal tissue.

The term “in vitro” and its grammatical equivalents as used herein refers to events that occur in an artificial environment, e.g., in a test tube or reaction vessel, in cell culture, etc., rather than within a multi-cellular organism.

The term “in vivo” and its grammatical equivalents as used herein refers to events that occur within a multi-cellular organism, such as a non-human animal, or a human animal.

The term “normal cells” and its grammatical equivalents as used herein refers to cells that undergo controlled cell division, controlled activation, or quiescent cells.

As used herein, a cancer can be a solid cancer such as a tumor, or a liquid cancer such as a blood related cancer.

The compounds herein are intended to include all isotopes of atoms occurring in the compounds herein. Isotopes include those atoms having the same atomic number but different mass numbers. By way of general example and without limitation, isotopes of hydrogen include deuterium (D) and tritium (T). Isotopes of carbon can include 13C, 14C, 15N, 31P, or 32P. Isotopically labeled compounds can generally be prepared using an appropriate isotopically labeled reagent in place of the non-labeled reagent otherwise employed. For example, methyl groups also include deuterated methyl groups such as —CD3.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

### Overview

Provided herein are compounds, compositions containing the compounds, and pharmaceutical compositions containing the compounds, and methods using these for treating a disease or condition in a subject. The compounds can be or comprise one or more compounds of Formula I, enantiomers of any of these, diastereomers of any of these, pharmaceutically acceptable salts of any of these, or deuterated derivatives of any of these. The compositions or pharmaceutical compositions can contain one or more of any of these. The disease or condition can be a cancer, for example in a tissue of the subject. The cancer can be comprised in a mammal, or contained in a tissue of a mammal, which can be a human, which can be male, or female.

Also provided herein are compounds, compositions containing the compounds, pharmaceutical compositions containing the compounds, and methods of using these for the treatment of Metastatic Castration Resistant Prostate Cancer (mCRPC) in a subject. The compounds can be or comprise one or more compounds of Formula I, enantiomers any of these, diastereomers any of these, pharmaceutically acceptable salts of any of these, or deuterated derivatives of any of these. The subject can be in need thereof of can be a mammal, a human, a female, or a male.

Also provided herein are compounds, compositions containing the compounds, pharmaceutical compositions containing the compounds, and methods of using these as an in situ vaccine for Metastatic Castration Resistant Prostate Cancer (mCRPC). The compounds can be or comprise one or more compounds of Formula I, enantiomers any of these, diastereomers any of these, pharmaceutically acceptable salts of any of these, or deuterated derivatives of any of these. The subject can be in need thereof of can be a mammal, a human, a female, or a male.

Also provided herein are compounds, compositions comprising the compounds, and pharmaceutical compositions comprising the compounds, and methods of making the compounds and compositions comprising the compounds, and methods of using these for targeted in situ vaccine delivery to a tumor. The compounds can be or comprise one or more compounds of Formula I, enantiomers thereof, diastereomers thereof, pharmaceutically acceptable salts of any of these, or deuterated derivatives of any of these. The subject in need thereof of can be a mammal, a human, a female, or a male.

Also provided herein are treatment regimens for the therapy of various diseases or conditions such as cancer in a subject. A treatment regime can comprise administering a compound of Formula I, enantiomers any of these, diastereomers of any of these, pharmaceutically acceptable salts of any of these, or deuterated derivatives of any of these. In some embodiments, the treatment regime further comprises radiotherapy. Briefly, further described herein are (1) radio-immunostimulant (RIMS) compounds; (2) compositions comprising RIMS compounds; (3) pharmaceutical compositions; (4) dosing; (5) methods of administration; (6) efficacy; (7) therapeutic applications; and (8) systems.

A subject herein can be a subject in need thereof, can be a mammal, can be a human, and can be a male or female. A subject herein can be diagnosed with a disease or condition prior to being treated, administered, or contacted with a compound of Formula I, an enantiomer or a diastereomer of any of the foregoing, a pharmaceutically acceptable salt of any of the foregoing, a deuterated derivative of any of the foregoing, or a composition or pharmaceutical composition comprising any of the foregoing. The diagnosis can be from an in vitro diagnostic, or an in vitro diagnostic which can be a companion diagnostic.

### Therapeutic Agents and Compounds

Compounds, for example of Formula I, therapeutic enantiomers or diastereomers of any of these, salts and pharmaceutically acceptable salts of any of these, and deuterated derivatives of any of these, can independently be administered continuously or discontinuously. When administered discontinuously, the administration can be at regularly spaced time intervals or irregularly spaced time intervals. Continuous and discontinuous administration can result in a baseline level of compound or agent being continuously present in a cell, tissue, organ, or system. The baseline level can be achieved, for example, for about: 1, 2, 3, 4, 5, 6, 7, 8, 9, 10, 11, 12, 13 14. 15 16. 17, 18, 19, 20, 21, 22, 23, 24, 25, 25, 27, 28, 29, 30, 31, 32, 33, 34, 35, 36, 37, 38, 39, 40, 41, 42, 43, 44, 45, 46, 47, 48, or more hours. Administration can independently be by any route of administration, and can be, for example, orally, intravenously, subcutaneous, intramuscular, intraperitoneal, intratumoral, intertumoral, administration to the brain or central nervous system, to the bladder, to an organ or portion thereof, to a tissue or portion thereof, or any combination of these. Compounds, for example of Formula I, agents, therapeutics enantiomers or diastereomers of any of these, salts and pharmaceutically acceptable salts of any of these, and deuterated derivatives of any of these, when administered as a solution, can independently have a concentration, for example, or about: 0.0001 μM, 0.001 μM, 0.01 μM, 0.1 μM 1.0 μM, 2.0 μM, 3.0 μM, 4.0 μM, 5.0 μM. 6.0 μM. 7.0 μM, 8.0 μM, 9.0 μM, 10 μM, 2 μM, 30 μM, 40 μM 50 μM, 60 μM, 70 μM, 80 μM, 90 μM, 100 μM, 200 μM, 300 μM 400 μM, 500 μM, or more.

In some instances, the compound of Formula I, any compound or agent or therapeutic herein, an enantiomer of any of the foregoing, a diastereomer of any of the foregoing, a salt or pharmaceutically acceptable salt of any of the foregoing, or a deuterated derivative of any of the foregoing can be delivered directly to a tissue, a tumor, or a cell with a system comprising, for example, a pump, for example a minipump or a syringe pump, and at least one or a needle, a hollow tube, and any combination thereof.

### Radio Immunostimulants (RIMS)

In some instances, the compound of Formula I, is a radio-immunostimulant (RIMS). As used herein, the terms “RIMS,” “RIMS compound,” and “RIMS conjugate” can be used interchangeably. In some embodiments, the RIMS is administered locally.

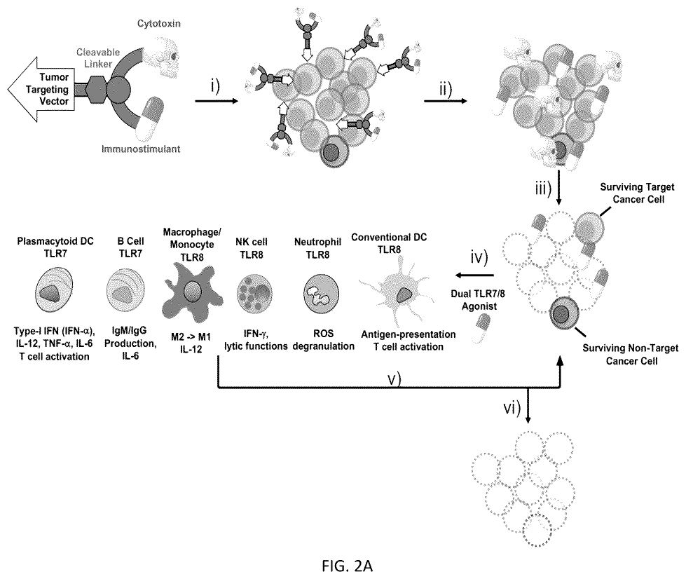

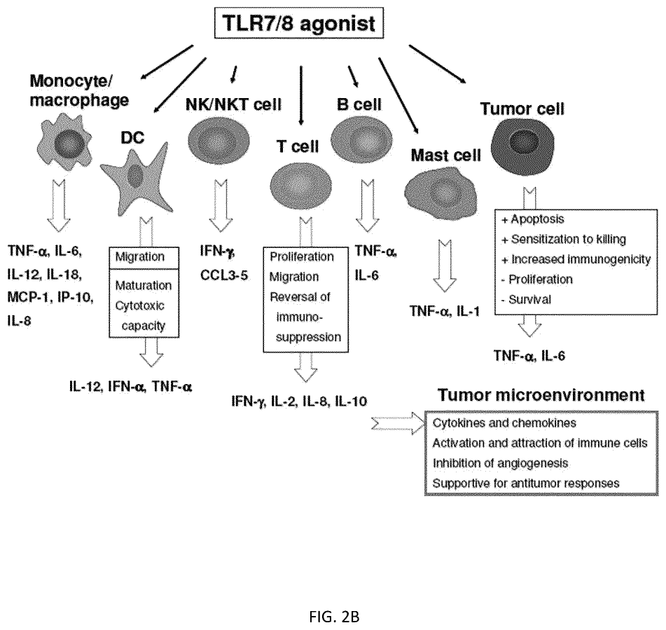

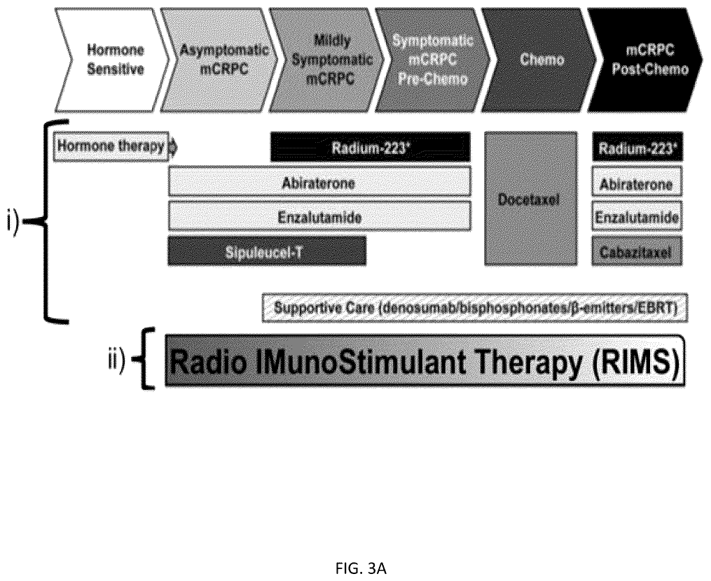

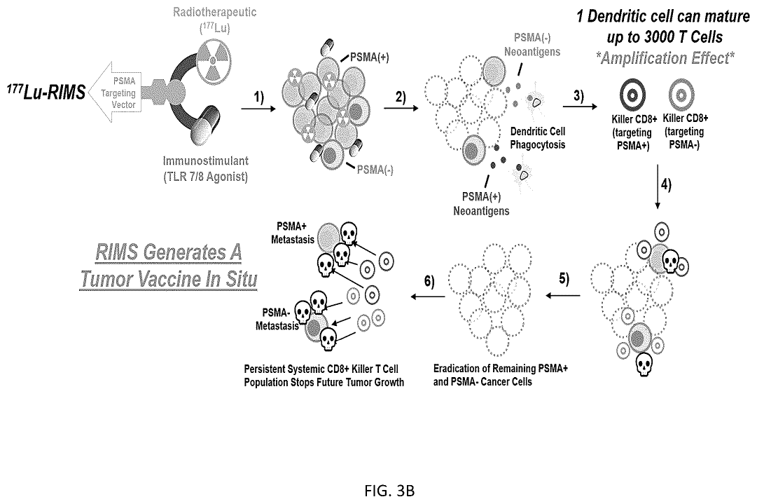

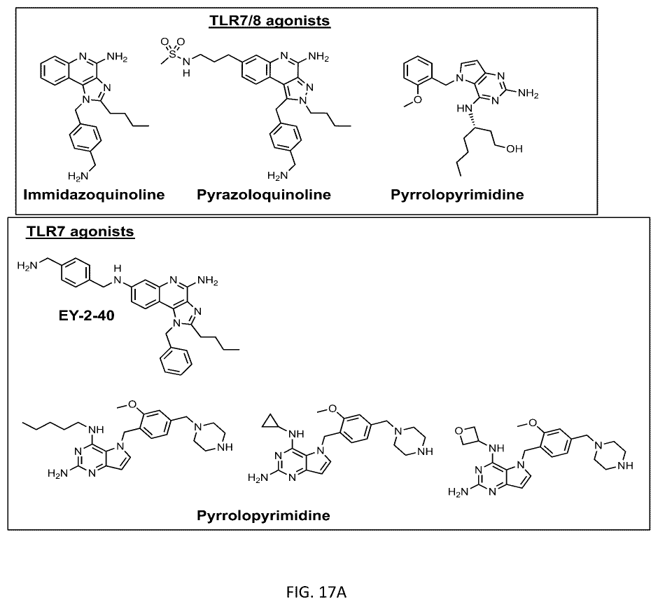

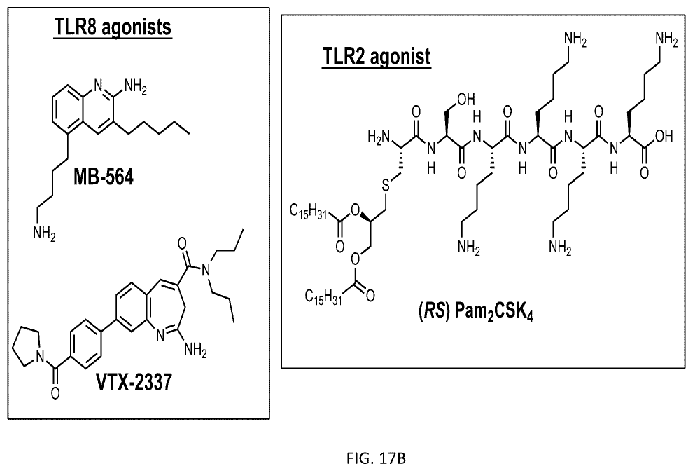

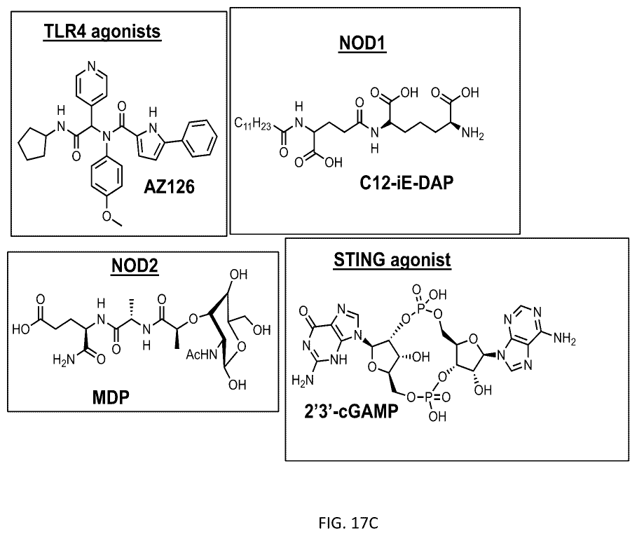

In some embodiments, a RIMS comprises an immunostimulant. In some embodiments, RIMS is a compound comprising an immunostimulant that is released in the tumor microenvironment. In some embodiments, the immunostimulant is a toll-like receptor (TLR) agonist. In particular, TLR agonists directly activate antigen presenting cells (APC's) resulting in enhanced humeral and cellular immune response, and Th1-biased responses, providing intense phagocytic activity. We will incorporate a potent dual TLR 7/8 small molecule agonist of the imidazoquinoline class of compounds with low nM activity. The proposed mechanism of action is as follows. Briefly, the localization/activation of immune cells and increased phagocytic activity catalyzed by the TLR 7/8 agonist combined with the apoptotic cancer cell debris generated by ¹⁷⁷Lu will result in tumor antigen loaded APC's (e.g., dendritic cells). This can cause recruitment of tumor specific cytotoxic T-cells that will kill cancer cells displaying those tumor antigens. In some embodiments, RIMS is a small molecule, or a molecular drug conjugate that incorporates: 1) A prostate specific membrane antigen (PSMA targeting motif; 2) The cytotoxic radiometal ¹⁷⁷Lu already employed in FDA-approved targeted radiotherapeutic applications (t112=6.65d, W=497 keV (78.6%), 384 keV (9.1%) and 176 keV (12.2%); and 3) A small molecule immunostimulant that is released in the tumor microenvironment. In some embodiments, RIMS is a small molecule immunostimulant that is released in the tumor microenvironment. In some embodiments, RIMS is used to treat heterogeneous tumors (PSMA+/−). In some embodiments, RIMS results in non-discriminatory phagocytosis of cancer cells via the localized immune cell population (e.g., macrophages) stimulated by a TLR agonist. In some embodiments, the immunostimulant is a TLR agonist. In some embodiments, the immunostimulant is a NOD-like receptor ligand. In some embodiments, the immunostimulant is a RIG-like receptor ligand. In some embodiments, the immunostimulant is a CLR ligand. In some embodiments, the immunostimulant is a CDS ligand. In some embodiments, the immunostimulant is a NOD-like receptor ligand. In some embodiments, the immunostimulant is an inflammasome inducer. In some embodiments, the immunostimulant is a STING agonist. In some embodiments, the immunostimulant is an immunostimulant described in FIGS. 17A-17C. In some embodiments, the immunostimulant is a TLR ligand. In some embodiments, the TLR ligand is TLR₂, TLR₃, TLR₄, TLR₅, TLR₆, TLR₇, TLR_{7/8}, TLR8, TLR₉, or TLR₁₀. In some embodiments the TLR agonist is a TLR7/8 agonist. In some embodiments, when cleaved, the TLR7/8 agonist interacts with a cell. In some embodiments, the TLR7/8 agonists, when cleaved, results in the production of a growth factor. In some embodiments, the TLR7/8, when cleaved results in the production of cytokines and chemokines in a tumor. In some embodiments, the TLR7/8, when cleaved, generates an anti-tumor response as shown in FIG. 2B. In some embodiments, the TLR7/8 agonist comprises: imidazoquinoline, pyrazoloquinoline, or pyrrolopyrimidine. In some embodiments, the TLR7/8 agonist is selected from the group consisting of: imidazoquinoline, pyrazoloquinoline, and pyrrolopyrimidine. In some embodiments the TLR agonist is a TLR7 agonist. In some embodiments, the TLR7 agonist comprises EY-2-40. In some embodiments, the TLR7 agonist comprises a pyrrolopyrimidine. In some embodiments, the TLR agonist is resiquimod. In some embodiments, the TLR agonist is imiquimod. In some embodiments the immunostimulant is cleaved after RIMS is exposed to endocytosis. In some embodiments, in some embodiments, a second cleavage event comprising the immunostimulant generates carbon dioxide. In some embodiments, one or more cleavage events to the RIMS generates a free, or unbound immunostimulant.

In some embodiments, RIMS comprises a ¹⁷⁷Lu complex. In some embodiments, the RIMS has a cytotoxic range of a p-radiometal. In some embodiments, the cytotoxic range is from about 50 to 2000 micrometers. In some embodiments, the cytotoxic range is from about: 50 to 2000, 100 to 1500, 200 to 1250, or 500 to 1000 micrometers. In some embodiments, the cytotoxic range is up to 2000 micrometers. In some embodiments, the cytotoxic range is at least to 500 micrometers. In some embodiments, the cytotoxic range is at least to 1000 micrometers. In some embodiments, administration of a RIMS results in phagocytosis by dendritic cells. In some embodiments, administration of a RIMS results in T-cell response stimulation.

In some embodiments, RIMS comprises cabazitaxel. In some embodiments, RIMS comprises a semi-synthetic derivative of the natural product taxol which is an antimitotic chemotherapeutic agent. In some embodiments, RIMS comprises enzalutamide or apalutamide which are structurally related nonsteroidal antiandrogens and synthetic analogues of diarylthiohydantoin. In some embodiments, RIMS comprises enzalutamide and apalutamide. Overall survival in the metastatic setting is extended by 4-5 months with enzalutamide. In some embodiments, RIMS comprises darolutamide which is a non-steroidal antiandrogen.

In some embodiments, RIMS comprises a cytotoxin. One common radiotherapy cytotoxin is Radium-223 (t112=11.4d, a=100 keV/μm) is an isotope of radium that behaves similar to calcium in the body, accumulating in bones (Bayer AG). This property is leveraged to treat patients with mCRPC with extensive bone metastasis. Radium-223 therapy improves overall survival to 14 months versus 11.2 months in patients treated with placebo. ¹⁷⁷Lu-PSMA-617 is a PSMA targeting agent that incorporates the radiotherapeutic lutetium isotope ¹⁷⁷Lu (t112=6.65d, W=497 keV (78.6%), 384 keV (9.1%) and 176 keV (12.2%)), that causes DNA strand breaks and eventually apoptosis. Originally developed by Endocyte and recently acquired by Novartis, ¹⁷⁷Lu-PSMA-617 is currently in clinically trials as a salvage therapy for patients with mCRPC. In some embodiments, ¹⁷⁷Lu-PSMA-617 is used as a positive control in comparison to RIMS.

In some embodiments, RIMS comprises an immunotherapeutic. In some embodiments, the immunotherapeutic is an immunostimulant. In some embodiments, the immunostimulant is a cancer vaccine. In some embodiments, introduction of a RIMS to a tumor results in tumor vaccine formation in situ. In some embodiments, the cancer vaccine targets dendritic cells. In some embodiments the cancer vaccine results in an adaptive immune response. In some embodiments, the immune response is against Prostatic Acid Phosphatase. Sipuleucel-T is an immunotherapeutic cancer vaccine used to treat patients with CRPC and mCRPC. It is unique amongst other approaches in that a patient's dendritic cells are isolated and incubated with a fusion protein containing antigen prostatic acid phosphatase, which is an antigen present in 95% of prostate cancer cells. The subsequent activated dendritic cells are then injected into the patient, resulting in the recruitment of killer T-cells that actively seek out and kill cancer cells that contain prostatic acid phosphatase. Sipuleucel-T provides a 4.1 month increase in overall survival compared to a placebo (25.8 months in the Sipuleucel-T group vs. 21.7 months in the placebo group).

In some embodiments, RIMS comprises a cytotoxic compound. In some embodiments, the cytotoxic compound is referred to herein as a cytotoxic payload or a cytotoxin. In some embodiments, the RIMS comprises a cytotoxin and an immunotherapeutic agent. In some embodiments, RIMS generates an adaptive immune response against prostate cancer when administered to a patient in need thereof. In some embodiments, the cytotoxin targets PSMA-expressing cells. In some embodiments, the cytotoxin partially binds to PSMA. In some embodiments, the cytotoxin binds to PSMA. In some embodiments RIMS compounds are screened for binding affinity to PSMA, labelling efficiency to ¹⁷⁷Lu, plasma stability and incubated with cathepsin enzyme to ensure release of the TLR 7/8 payload.

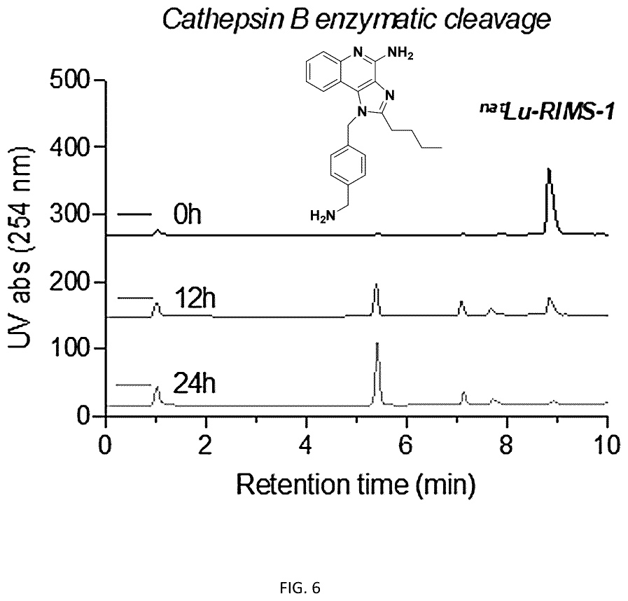

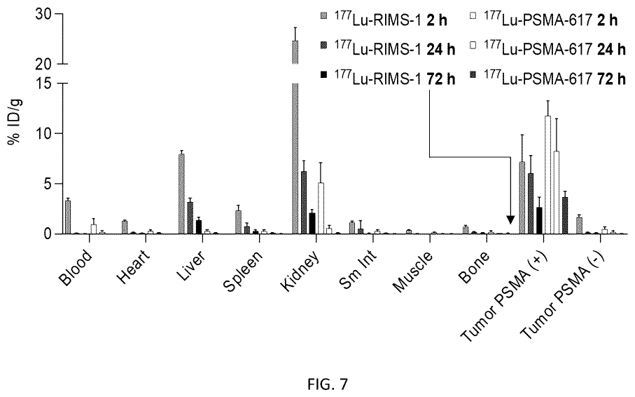

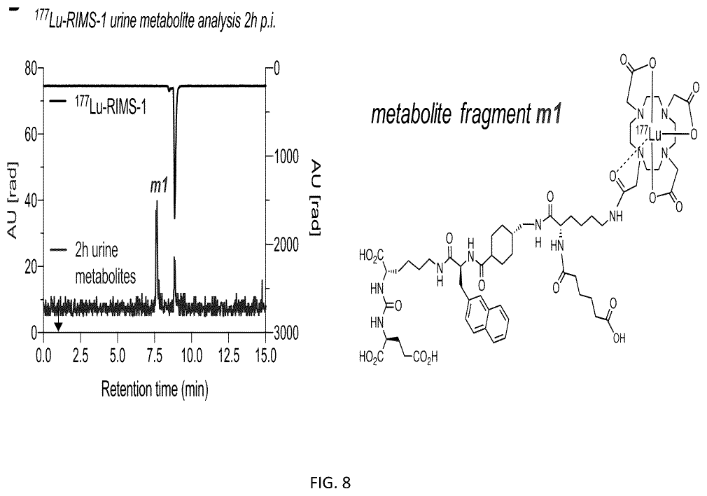

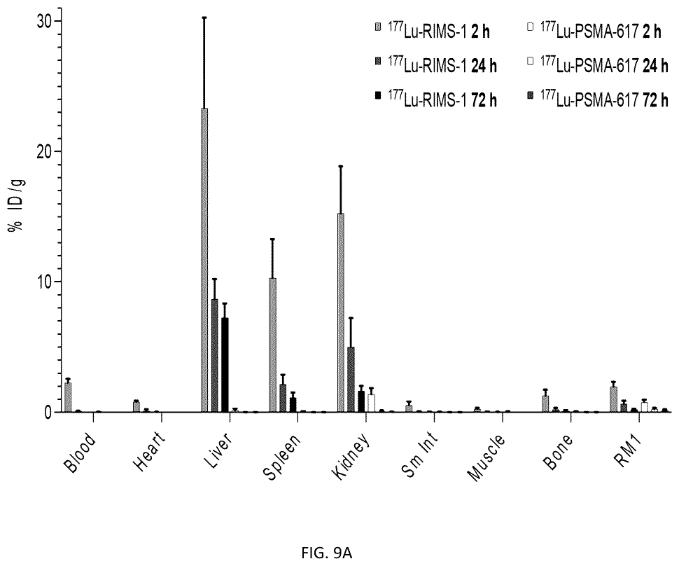

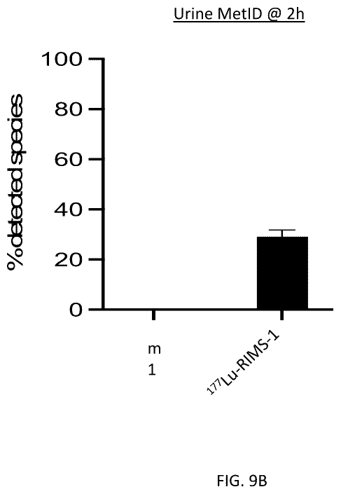

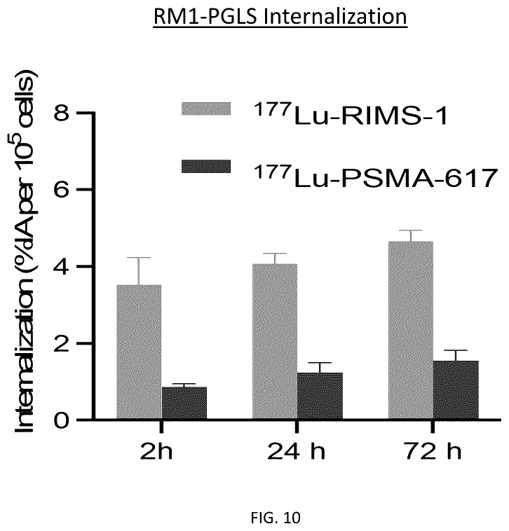

In some embodiments, RIMS comprises a linker. In some embodiments, the linker comprises a hydrazone. In some embodiments, the linker comprises a peptide. In some embodiments, the linker comprises a dipeptide. In some embodiments, the linker comprises a disulfide. In some embodiments, the linker comprises a sugar. In some embodiments, the linker comprises a carbohydrate. In some embodiments, the linker is a chemically cleavable linker. A chemically cleavable linker, as used herein can include a cleavage of the linker moiety from a RIMS, wherein the cleavage is generated by a small molecule, solvent, chemical reagent, or a combination thereof. In some embodiments, the linker is an enzymatically cleavable linker. In some embodiments, the linker undergoes cleavage in vitro. In some embodiments, the linker undergoes cleavage in vivo. In some embodiments, the linker undergoes a homolytic cleavage. In some embodiments, the linker undergoes a heterolytic cleavage. An enzymatically cleavable linker, as used herein can include a cleavage of the linker moiety from a RIMS, wherein the cleavage is generated by an enzyme, or at least one enzyme. In some embodiments, the linker can comprise one or more covalent bonds that are cleavable by an enzyme. In some embodiments, the enzyme is a beta-glucoronidase. In some embodiments, the enzyme is a protease. In some embodiments, the enzyme comprises a cathepsin enzyme. In some embodiments, the enzyme is cathepsin B. In some embodiments, cathepsin B mediated cleavage of an immunostimulant is observable by UV/Vis spectrophotometry (e.g., see FIG. 6). In some embodiments, a linker undergoes cleavage by proteolysis. In some embodiments, the linker can comprise one or more covalent bonds that are cleavable by a small molecule, chemical reagent, solvent, or buffer. In some embodiments, the linker is a pH sensitive linker. In some embodiments, the linker undergoes cleavage by acid catalyzed hydrolysis. In some embodiments, the linker undergoes cleavage by base catalyzed hydrolysis. In some embodiments, the linker is an albumin binding linker. In some embodiments, the linker is a branched PEG linker. In some embodiments, the linker comprises a polar molecule. In some embodiments, the linker comprises a polar neutral molecule. In some embodiments, the linker comprises a neutral molecule. In some embodiments, the linker comprises a zwitterionic molecule. In some embodiments, the linker comprises a positively charged molecule. In some embodiments, the linker comprises a negatively charged molecule.

In some embodiments, the linker is covalently bonded to an immunostimulant. In some embodiments, the linker is covalently bonded to a cytotoxin. In some embodiments, the linker is covalently bonded to an immunostimulant and covalently bonded to a cytotoxin. In some embodiments, cleavage of a linker described herein results in the release of an immunostimulant from the RIMS.

### Pharmaceutical Compositions

Also provided herein is a pharmaceutical composition comprising the compound of Formula I, the diastereomer or the enantiomer of any of the foregoing, or the pharmaceutically acceptable salt of any of the foregoing, or the deuterated derivative of any of the foregoing; and a pharmaceutically acceptable: excipient, diluent, or carrier. The pharmaceutical composition can be in unit dose form. Additionally, the pharmaceutical composition can comprise an additional active agent or pharmaceutically acceptable salt thereof or prodrug thereof. Further, the pharmaceutical composition can be in the form of a powder, a tablet, a capsule, a liquid, or a gel.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, United States Pharmacopeia (U.S.P.), and isotonic sodium chloride solution. In addition, sterile, fixed oils are employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Exemplary carriers and excipients can include dextrose, sodium chloride, sucrose, lactose, cellulose, xylitol, sorbitol, malitol, gelatin, polymers, polyethylene glycol (PEG), polyvinylpyrrolidone (PVP), and any combination thereof. In some embodiments, an excipient such as dextrose or sodium chloride can be independently at a percent from about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or up to about 15% by weight of the total weight of the composition or pharmaceutical composition.

In some embodiments, in the pharmaceutical composition, the compound of Formula I, the compound; or the enantiomer or the diastereomer of any of the foregoing; or the pharmaceutically acceptable salt of any of the foregoing, or the deuterated derivative of any of the foregoing, is present in the pharmaceutical composition in an amount ranging from about 0.001 mg to about 25,000 mg, for example from about: 0.0001 mg to 1 mg, 0.001 mg to 2 mg, 0.01 mg to 4 mg, 0.1 mg to 5 mg, 1 mg to 10 mg, 5 mg to 15 mg, 50 mg to 500 mg, 100 mg to 750 mg, 1000 mg to 10000 mg, 5000 mg to 15000 mg, 10000 mg to 25000 mg. In some embodiments, the additional active agent or pharmaceutically acceptable salt thereof or prodrug thereof can be independently present in the pharmaceutical composition in an amount ranging from about 0.001 mg to about 25,000 mg for example from about: 0.0001 mg to 1 mg, 0.001 mg to 2 mg, 0.01 mg to 4 mg, 0.1 mg to 5 mg, 1 mg to 10 mg, 5 mg to 15 mg, 50 mg to 500 mg, 100 mg to 750 mg, 1000 mg to 10000 mg, 5000 mg to 15000 mg, 10000 mg to 25000 mg.

In some embodiments is provided a kit comprising a compound therein, a diastereomer thereof, an enantiomer thereof, a pharmaceutically acceptable salt thereof, or a deuterated derivative thereof, or a pharmaceutically composition therein, and a container. In some embodiments are pharmaceutical compositions described herein and a container. In some embodiments, the container is a syringe. In some embodiments, the container is an intravenous (IV) bag. In some embodiments, the container is disposable. In some embodiments, the container is recyclable. In some embodiments, the container is a single use container. In some embodiments, the container is resealable.

In some embodiments is provided a method of treating a disease or condition in a subject. In some embodiments, the disease or condition is a cancer. In some embodiments, the method comprises administering a therapeutically effective amount of the pharmaceutical composition herein to the subject, who can be a subject in need thereof, thereby treating the disease or the condition, which can be a cancer. In some embodiments is provided a method of treating a disease or condition in a subject, who can be subject in need thereof, the method comprising administering the compound of Formula I, the diastereomer or the enantiomer of any of the foregoing, or the pharmaceutically acceptable salt of any of the foregoing, or the deuterated derivative of any of the foregoing; in a therapeutically effective amount to the subject, thereby treating the disease or condition, which can be a cancer. In some embodiments, the administering is selected from the group consisting of: oral, an injection; subcutaneous, intra-tumoral; systemic, local, intravenous, intraperitoneal, intramuscular, and any combination thereof. In some embodiments, the subject can be a mammal. In some embodiments, the subject can be a human. In some embodiments, the subject can be a male. In some embodiments, the subject can be a female.

In some embodiments, in a method herein, the administering or the contacting can be: as needed, once per day, twice per day, three times per day, once per week, once per two weeks, once per three weeks, once per month, once every six months, once per year, or for life. In some embodiments, an effective or a therapeutically effective amount can range from about 0.001 mg to about 25,000 mg, for example from about: 0.0001 mg to 1 mg, 0.001 mg to 2 mg, 0.01 mg to 4 mg, 0.1 mg to 5 mg, 1 mg to 10 mg, 5 mg to 15 mg, 50 mg to 500 mg, 100 mg to 750 mg, 1000 mg to 10000 mg, 5000 mg to 15000 mg, 10000 mg to 25000 mg.of a compound herein, an enantiomer or a diastereomer thereof, a pharmaceutically acceptable salt of an of these, or a deuterated derivative of any of these, or of a pharmaceutical composition herein, which can optionally be in unit dose form.

In some embodiments, also are provided methods of making and testing compounds of Formula I, enantiomers and diastereomers of any of these, salts, and pharmaceutically acceptable salts of any of these, and deuterated derivatives of any of these.

### General Methods of Making Pharmaceutical Compounds

The compounds of Formula (I) can be provided as amorphous solids or crystalline solids. Lyophilization can be employed to provide the compounds as amorphous solids. It should further be understood that solvates (e.g., hydrates) of the compounds are also contemplated herein. The term “solvate” can mean a physical association of a compound with one or more solvent molecules, whether organic or inorganic. This physical association includes or can include hydrogen bonding. In certain instances, the solvate can be capable of isolation, for example when one or more solvent molecules are incorporated in the crystal lattice of the crystalline solid. “Solvate” encompasses both solution-phase and isolable solvates. Exemplary solvates can include hydrates, ethanolates, methanolates, isopropanolates, acetonitrile solvates, and ethyl acetate solvates. In addition, compounds, subsequent to their preparation, can be isolated and purified to obtain a composition containing an amount by weight equal to or greater than 99% of a compound of Formula (I) (“substantially pure”), which is then used or formulated as described herein. Such “substantially pure” compounds are also contemplated herein. Compounds can be prepared in several ways and can be synthesized using the methods described herein. The reactions and techniques described herein are performed in solvents appropriate to the reagents and materials employed and are suitable for the transformations being affected. Also, in the description of the synthetic methods described below, it is to be understood reaction conditions, including choice of solvent, reaction atmosphere, reaction temperature, duration of the experiment and work up procedures, can be chosen to be the conditions standard for that reaction. It is understood by one skilled in the art of organic synthesis that the functionality present on various portions of the molecule must be compatible with the reagents and reactions proposed.

### Additional Treatments

In some embodiments, the methods provided herein comprise administering at least one additional treatment to a subject. In some embodiments, the additional treatment is surgery. In some embodiments, the additional treatment is radiation therapy.

In some embodiments, the additional treatment is an additional therapeutic agent. In some embodiments, the methods provided herein comprise administering an additional agent in combination with a RIMS compound. In some embodiments, the additional agent is a cell-death inducing agent. In some embodiments, the additional agent is an anti-cancer agent. In some embodiments, the anti-cancer agent is a chemotherapeutic agent. A chemotherapeutic agent or compound is any agent or compound useful in the treatment of cancer. The chemotherapeutic cancer agents that can be used in combination with a RIMS compound provided herein which include, but are not limited to, mitotic inhibitors (e.g., vinca alkaloids). These include vincristine, vinblastine, vindesine and Navelbine™ (e.g., vinorelbine, 5′-noranhydroblastine). In yet other cases, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

The disclosed agents provided herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the RIMS compounds provided herein can include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bevacizumab; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; folinic acid; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; erythrocyte gene therapy; velaresol; veramine; verdins; verteporfin; vinorelbine; vinxaltine; vitaxin; vorozole; zanoterone; zeniplatin; zilascorb; and zinostatin stimalamer. Any of the aforementioned chemotherapeutics can independently be administered at a clinically effective dose. A chemotherapeutic can also independently be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14 after administration of an agent provided herein. In some cases, a subject can have a refractory cancer that is unresponsive to a chemotherapeutic.

### Methods of Administering a Composition Comprising a RIMS Compound

Provided herein can be methods for administering a therapeutic regime to a subject having a disease or disorder (e.g., cancer). In some embodiments, the administering is sustained administration of a therapeutically effective amount of a RIMS compound. In some embodiments, the sustained administration of the RIMS compound comprises providing to a tissue the RIMS compound in an amount sufficient to achieve a distribution of at least about 10 ng/mm² within said tissue for a period of at least 4 hours, thereby generating an immune response in the tissue. In some embodiments, the sustained administration further forms a gradient of a sub-therapeutic amount of the RIMS compound adjacent to an administration site within the tissue. In some embodiments, sustained administration of RIMS compound comprises additional administration steps. In some embodiments, the RIMS compound is administered more than once. In some embodiments, the administering is via a system provided herein. In some embodiments, the administering is local administration within a tissue. In some embodiments, the tissue is contacted in vivo with an effective amount of RIMS compound one time. In some embodiments, the administering is local administration or systemic administration. In some embodiments, the administering or contacting step is via intratumoral injection, oral administration, transdermal injection, inhalation, nasal administration, topical administration, vaginal administration, ophthalmic administration, intracerebral administration, rectal administration. In some embodiments, the administering or contacting step is via intratumoral injection.

In some embodiments, the RIMS is administered to a patient in need thereof undergoing radiotherapy. In some embodiments, administration of a RIMS compound results in the release of an immunostimulant in vitro. In some embodiments, administration of a RIMS compound results in the release of an immunostimulant in vivo. In some embodiments, the immunostimulant is a toll-like receptor agonist. In some embodiments, the immunostimulant is a pattern recognition receptor. In some embodiments, the immunostimulant is activated by pathogen associated molecular pattern (PAMP) recognition in vitro. In some embodiments, the immunostimulant is activated by pathogen associated molecular pattern (PAMP) recognition in vivo.

In some instances, an agent or combination of agents (e.g., a RIMS compound) provided herein are administered as a unit dosage form. Many agents can be administered orally as liquids, capsules, tablets, or chewable tablets. Because the oral route is the most convenient and usually the safest and least expensive, it is the one most often used. However, it has limitations because of the way a drug typically moves through the digestive tract. For agents administered orally, absorption may begin in the mouth and stomach. However, most agents are usually absorbed from the small intestine. The drug passes through the intestinal wall and travels to the liver before being transported via the bloodstream to its target site. The intestinal wall and liver chemically alter (metabolize) many agents, decreasing the amount of drug reaching the bloodstream. Consequently, these agents are often given in smaller doses when injected intravenously to produce the same effect.

In some cases, a treatment regime may be dosed according to a body weight of a subject. In subjects who are determined obese (BMI>35) a practical weight may need to be utilized. BMI is calculated by: BMI=weight (kg)/[height (m)]².

In some cases, a therapeutic regime can be administered along with a carrier or excipient. RIMS compounds provided herein can be administered with one or more of a second agent, sequentially, or concurrently, either by the same route or by different routes of administration. When administered sequentially, the time between administrations is selected to benefit, among others, the therapeutic efficacy and/or safety of the combination treatment. By way of example and not limitation, the time between administrations is about 1 hr, about 2 hr, about 4 hr, about 6 hr, about 12 hr, about 16 hr or about 20 hr. In certain embodiments, the time between administrations is about 1, about 2, about 3, about 4, about 5, about 6, or about 7 more days. In some embodiments, the time between administrations is about 1 week, 2 weeks, 3 weeks, or 4 weeks or more. In some embodiments, the time between administrations is about 1 month or 2 months or more.

In some embodiments, RIMS compound provided herein contact the mammalian tissue for at least about 4 hours, at least about 6 hours, at least about 10 hours, at least about 12 hours, at least about 14 hours, at least about 16 hours, at least about 18 hours, at least about 20 hours, at least about 22 hours, at least about 24 hours, at least about 26 hours, at least about 28 hours, at least about 30 hours, at least about 36 hours, at least about 48 hours, up to 72 hours. In some embodiments, a RIMS compound provided herein contact the mammalian tissue for about 4 hours. In some embodiments, a RIMS compound provided herein contact the mammalian tissue for about 6 hours. In some embodiments, a RIMS compound provided herein contact the mammalian tissue for about 10 hours. In some embodiments, a RIMS compound provided herein contact the mammalian tissue for about 12 hours. In some embodiments, a RIMS compound provided herein contact the mammalian tissue for about 24 hours. In some embodiments, a RIMS compound provided herein contact the mammalian tissue for about 48 hours. In some embodiments, a RIMS compound provided herein contact the mammalian tissue for about 72 hours.

In certain embodiments, the amount and frequency of administration of the second agent can used standard dosages and standard administration frequencies used for the particular compound.

### Dosing and Tissue Distribution

The methods provided herein comprise administering to a subject an agent or pharmaceutical composition provided herein in an amount effective to initiate an immune response in a tissue in vivo. Agents and pharmaceutical compositions for administering to a subject in need thereof may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. The animal model may also be used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices may be useful in some embodiments. The data obtained from cell culture assays and animal studies may be used in formulating a range of dosage for human use.

A typical human dose of an agent provided herein (e.g., a RIMS compound) may be from about 10 μg/kg body weight/day to 10,000 mg/kg/day wherein mg/kg/day refers to milligrams of an agent described herein per kilogram of subject body weight per day. In some embodiments, the dose of an agent provided herein is from about 0.1 mg/kg to about 1000 mg/kg, from 1 mg/kg to 1000 mg/kg, 1 mg/kg to 800 mg/kg, from about 1 mg/kg to about 700 mg/kg, from about 2 mg/kg to about 500 mg/kg, from about 3 mg/kg to about 400 mg/kg, 4 mg/kg to about 300 mg/kg, or from about 5 mg/kg to about 200 mg/kg, wherein mg/kg refers to milligrams of an agent described herein per kilogram of subject body weight. In certain embodiments, the suitable dosages of the agent can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 2,000 mg/kg, 3,000 mg/kg, 4,000 mg/kg, 5,000 mg/kg, 6,000 mg/kg, 7,000/mg/kg, 8,000 mg/kg, 9,000 mg/kg, up to 9,600 mg/kg, wherein mg/kg refers to milligrams of an agent described herein per kilogram of subject body weight. In some embodiments, the dose of an agent provided herein is from about 100 mg/kg/day to about 6,400 mg/kg/day four times per day. In some embodiments, the dose of an agent provided herein is from about 50 mg/kg/day to about 25 mg/kg/day, wherein mg/kg/day refers to milligrams of an agent described herein per kilogram of subject body weight per day. In some embodiments, the dose of an agent provided herein is from about 400 mg/kg/day to about 800 mg/kg/day. In certain embodiments, the dose of the agent can be administered once per day or divided into sub-doses and administered in multiple doses, e.g., twice, three times, or four times per day.

In some embodiments, agents provided herein are administered in an amount of at least: about 10 nanograms (ng) or more, about 20 ng or more, about 30 ng or more, about 40 ng or more, about 50 ng or more, about 60 ng or more, about 70 ng or more, about 80 ng or more, about 90 ng or more, up to 100 ng. In some embodiments, the agent is administered in an amount of at least: about 1 microgram (μg) or more, about 5 μg or more, about 10 μg or more, about 20 μg or more, about 30 μg or more, about 40 μg or more, about 50 μg or more, about 60 μg or more, about 70 μg or more, about 80 μg or more, about 90 μg or more, up to 100 μg.

In some embodiments, agents provided herein are administered at a concentration of at least: about 0.1 micromolar (μM) or more, about 1 μM or more, about 2 μM or more, about 3 μM or more, about 4 μM or more, about 5 μM or more, about 6 μM or more, about 7 μM or more, about 8 μM or more, about 9 μM or more, about 10 μM or more, about 15 μM or more, about 20 μM or more, about 25 μM or more, about 30 μM or more, about 35 μM or more, about 40 μM or more, about 45 μM or more, about 50 μM or more, about 55 μM or more, about 60 μM or more, about 65 μM or more, about 70 μM or more, about 75 μM or more, about 80 μM or more, about 85 μM or more, about 90 μM or more, about 95 μM or more, about 100 μM or more, about 110 μM or more, about 120 μM or more, about 130 μM or more, about 140 μM or more, about 150 μM or more, about 160 μM or more, about 170 μM or more, about 180 μM or more, about 190 μM or more, about 200 μM or more, about 300 μM or more, about 400 μM or more, about 500 μM or more, up to 1 mM. In some embodiments, agents provided herein are administered at a concentration of at least: about 0.1 μM up to about 500 μM. In some embodiments, agents provided herein are administered at a concentration of at least about: 1 μM up to 500 μM. In some embodiments, agents provided herein are administered at a concentration of at least about: 0.1 μM up to 10 μM. In some embodiments, agents provided herein are administered at a concentration of at least about: 1 μM up to 10 μM.

### Efficacy

Therapeutic efficacy of an agent and/or pharmaceutical composition provided herein may be determined by evaluating and comparing patient symptoms and quality of life pre- and post-administration. Such methods apply irrespective of the mode of administration. In some embodiments, pre-administration refers to evaluating patient symptoms and quality of life prior to onset of therapy and post-administration refers to evaluating patient symptoms and quality of life at least 1, 2, 3, 4, 5, 6, 7, 8, 9, 10, 11, 12, 13, 14, 15, 16, 17, 18, 19, or 20 weeks after onset of therapy. In some embodiments, pre-administration refers to evaluating patient symptoms and quality of life prior to onset of therapy and post-administration refers to evaluating patient symptoms and quality of life of up to 52 weeks after onset of therapy. In a particular embodiment, the post-administration evaluating is performed about: 2-8, 2-6, 4-6, or 4 weeks after onset of therapy. In a particular embodiment, patient symptoms (e.g., symptoms related to cancer, fibrosis, or autoimmune disease) and quality of life pre- and post-administration are evaluated clinically and by questionnaire assessment.

The agents and methods provided herein can be used to reduce cancer cell proliferation or survival in vivo or in vitro. Methods of evaluating tumor progression or cell proliferation are known in the art. In some embodiments, overall response is assessed from time-point response assessments (based on tumor burden) as follows:

Complete Response (CR): Disappearance of all target lesions. Any pathological lymph nodes (whether target or non-target) must have reduction in short axis to <10 mm.

Partial Response (PR): At least a 30% decrease in the sum of diameters of target lesions, taking as reference the baseline sum diameters.

Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).

Stable Disease (SD): Neither sufficient shrinkage to qualify for PR nor sufficient increase to qualify for PD, taking as reference the smallest sum diameters while on study.

In some embodiments, an in vitro cell proliferation assay is used to assess the efficacy of a one or more RIMS compounds provided herein. The compositions and methods provided herein result in a reduction in the proliferation or survival of a plurality of cells. For example, after treatment with one or more of the agents provided herein, cell proliferation or survival is reduced by 5% or greater (e.g., 10%, 20%, 30%, 40%, 50%, 60%, 70%, 80%, 90% or greater) relative to cell proliferation or survival prior to treatment.

In some embodiments, animal models are used to assess the efficacy of a one or more RIMS compounds provided herein in vivo. The RIMS compounds and methods provided herein can result in a reduction in size or volume of a hyperproliferating tissue (e.g., a tumor). For example, after treatment, tissue size is reduced by 5% or greater (e.g., 10%, 20%, 30%, 40%, 50%, 60%, 70%, 80%, 90% or greater) relative to its size prior to treatment. Size of a tissue (e.g., a tumor) may be measured by any reproducible means of measurement. The size of a tissue may be measured as a diameter of the tumor or by any reproducible means of measurement.

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. The number of metastatic nodules may be measured by counting metastatic nodules visible to the naked eye or at a specified magnification (e.g., 2×, 10×, or 50×).

Treating a disease or disorder (e.g., cancer) can result in an increase in average survival time of a population of subjects treated according to the present disclosure in comparison to a population of untreated subjects. For example, the average survival time is increased by more than 30 days (more than 60 days, 90 days, 120 days or longer). An increase in average survival time of a population may be measured by any reproducible means. An increase in average survival time of a population may be measured, for example, by calculating for a population the average length of survival following initiation of treatment with the compound of the disclosure. An increase in average survival time of a population may also be measured, for example, by calculating for a population the average length of survival following completion of a first round of treatment with the compound of the disclosure.

Treating a disease or disorder (e.g., cancer) can also result in a decrease in the mortality rate of a population of treated subjects in comparison to an untreated population. For example, the mortality rate is decreased by more than 2% (e.g., more than 5%, 10%, 25%, or greater). A decrease in the mortality rate of a population of treated subjects may be measured by any reproducible means, for example, by calculating for a population the average number of disease-related deaths per unit time following initiation of treatment with the compound of the disclosure. A decrease in the mortality rate of a population may also be measured, for example, by calculating for a population the average number of disease-related deaths per unit time following completion of a first round of treatment with a RIMS compound.

Treating a disease or disorder can also result in a decrease in at least one symptom associated with the disease, disorder, or condition. In some embodiments, the methods provided herein reduce at least one symptom of a disease or disorder by at least 10%, 20%, 30%, 40%, 50%, 70%, 80%, 90% or greater relative to number prior to treatment. In some embodiments, following contact with a mammalian tissue or administration of a RIMS compound, cell death can be detected at a time point at or after contacting the mammalian tissue with the RIMS compound. In some embodiments, the methods provided herein increase cell death by at least 5%, 10%, 20%, 30%, 40%, 50%, 60%, 70%, 80%, 90% or greater relative to number prior to treatment.

### Therapeutic Applications

Provided herein are methods of treating a cancer in a subject. In some embodiments, the subject has, is suspected of having, or is at risk of developing a hyperproliferative disease or condition. In some embodiments, methods provided herein further comprise a step of obtaining a biopsy of the tissue for histological analysis.

In some embodiments, the subject has, is suspected of having, or is at risk of developing cancer. In some embodiments, the subject has a benign tumor. In some embodiments, the subject has a pre-cancerous lesion. In some embodiments, the subject has received a previous treatment to treat the cancer. In some embodiments, the cancer is a prostate cancer. In some embodiments, the prostate cancer is a metastatic prostate cancer. In some embodiments, the cancer is mCRPC.

In some embodiments, a compound described herein is used as a cancer vaccine. In some embodiments, a compound described herein is used for the treatment of a tumor. In some embodiments the tumor is a heterogeneous tumor. In some embodiments, a compound described herein generated an adaptive immune response against a heterogeneous disease.

In some embodiments, treatments described herein comprise an immunotherapy. In some embodiments, treatments described herein comprise administration of a cytotoxin. In some embodiments, treatments described herein comprise administration of an antiandrogen. In some embodiments, treatments described herein comprise administration of a cytotoxin and an antiandrogen.

In some embodiments, RIMS is a small molecule immunostimulant that is released in the tumor microenvironment. In some embodiments, administration of a RIMS results in T-cell response stimulation towards PSMA(−) cells. In some embodiments, administration of a RIMS results in T-cell response stimulation towards PSMA(+) cells. In some embodiments, administration of a RIMS promotes targeted, distal tumor cell death against PSMA(−) cells that have detached from the original heterogenous tumor population and can prevent a major resistance pathway in prostate cancer. In some embodiments, RIMS is used to treat heterogeneous tumors (PSMA+/−). In some embodiments, RIMS results in non-discriminatory phagocytosis of cancer cells via the localized immune cell population (e.g., macrophages) stimulated by a TLR agonist. In some embodiments, the TLR agonist is resiquimod. In some embodiments, the TLR agonist is imiquimod.

### EXEMPLARY EMBODIMENTS

1. A compound of Formula I:

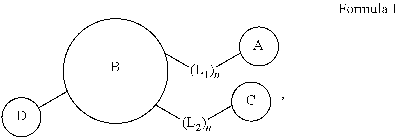

a diastereomer or enantiomer thereof, or a pharmaceutically acceptable salt of any of the foregoing, or a deuterated derivative or any of the forgoing, or a radioisotope of any of the forgoing,
wherein:
A, L₁, B, D, L₂, and C groups are each independently covalently connected as one molecule;
A comprises a: taxoid, vinca alkaloid, anthracycline, maytansinoid, tubulysin, auristatin, exatecan, duocarmycin, Seco-Cyclopropabenzindol-4-One dimer or monomer, pyrrolobenzodiazepine dimer or monomer, hemiasterlin, ²¹²Pb-DOTA, ¹⁷⁷Lu-DOTA (1,4,7,10-Tetraazacyclododecane-1,4,7,10-tetraacetic acid), ⁹⁰Y-DOTA, ²²⁵Ac-DOTA, ⁴⁷Sc-DOTA, ⁶⁷Cu-DOTA, ¹³¹I-L-Tyrosine, or any combination thereof;
when A is ¹⁷⁷Lu-DOTA (1,4,7,10-Tetraazacyclododecane-1,4,7,10-tetraacetic acid), ⁹⁰Y-DOTA, ²¹²Pb-DOTA, ²²⁵Ac-DOTA, ⁴⁷Sc-DOTA, ⁶⁷Cu-DOTA, ¹³¹I-L-Tyrosine, or any combination thereof, A is covalently bound to L₁ by a C—C or C—N bond;
L₁ and L₂ are each independently selected from: (CH₂)ₙ, a dipeptide, a tripeptide, a disulfide, a hydrazone, a beta glucan,

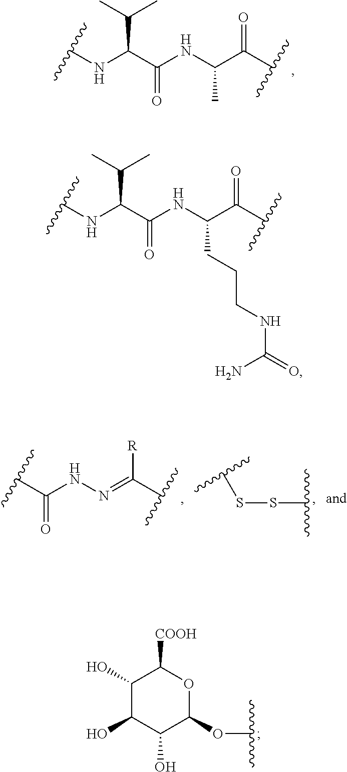

R is an aliphatic or unsaturated C1-C10, or an aromatic C6 or C10 group, wherein each R is optionally substituted with an amide, an ester, a C3-C10 cycloalkyl, an ether, or C1-C4 group;
each n is independently an integer 0-8;
B is selected from the group consisting of:

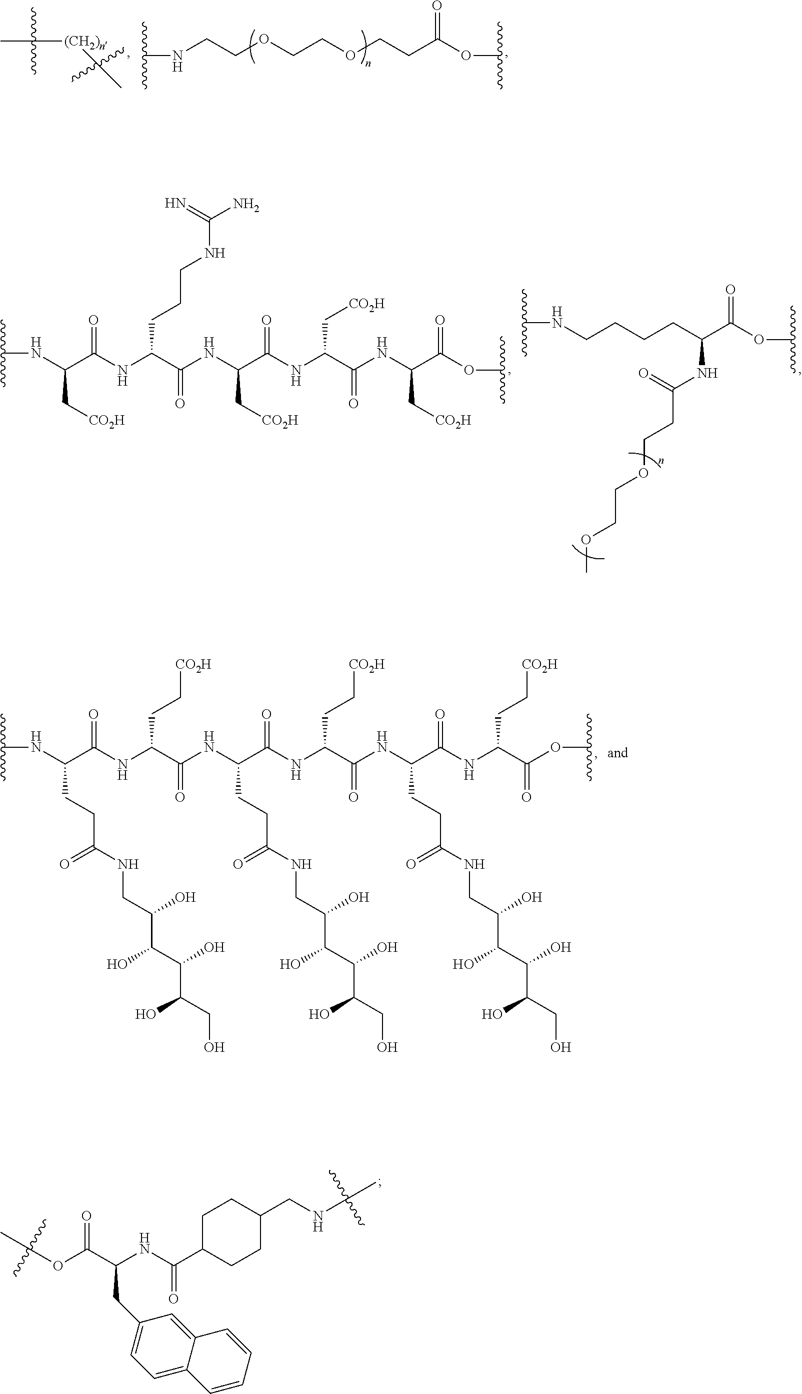

C is an immunostimulant toll-like receptor (TLR) ligand comprising a: TLR2 agonist, TLR3 agonist, TLR4 agonist, TLR5 agonist, TLR6 agonist, TLR7 agonist, TLR7/8 agonist, TLR8 agonist, TLR9 agonist, TLR10 agonist, nucleotide oligomerization domaine (NOD)-like receptor ligand, retinoic acid-inducible (RIG)-like receptor ligand, C-type lectin receptor (CLR) ligand, a cytosolic dsDNA sensor (CDS) ligand, inflammasome inducer or stimulator of interferon genes (STING) agonist,

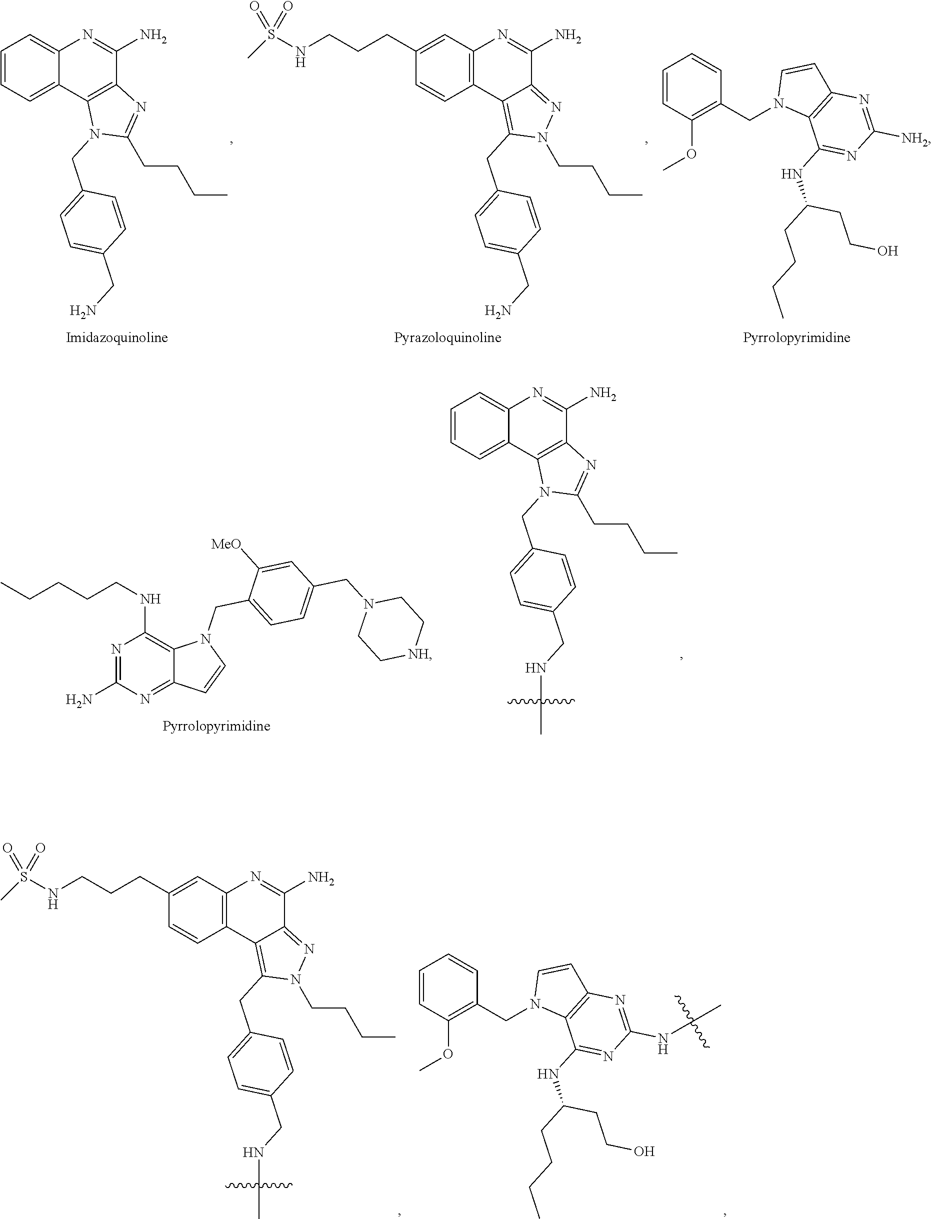

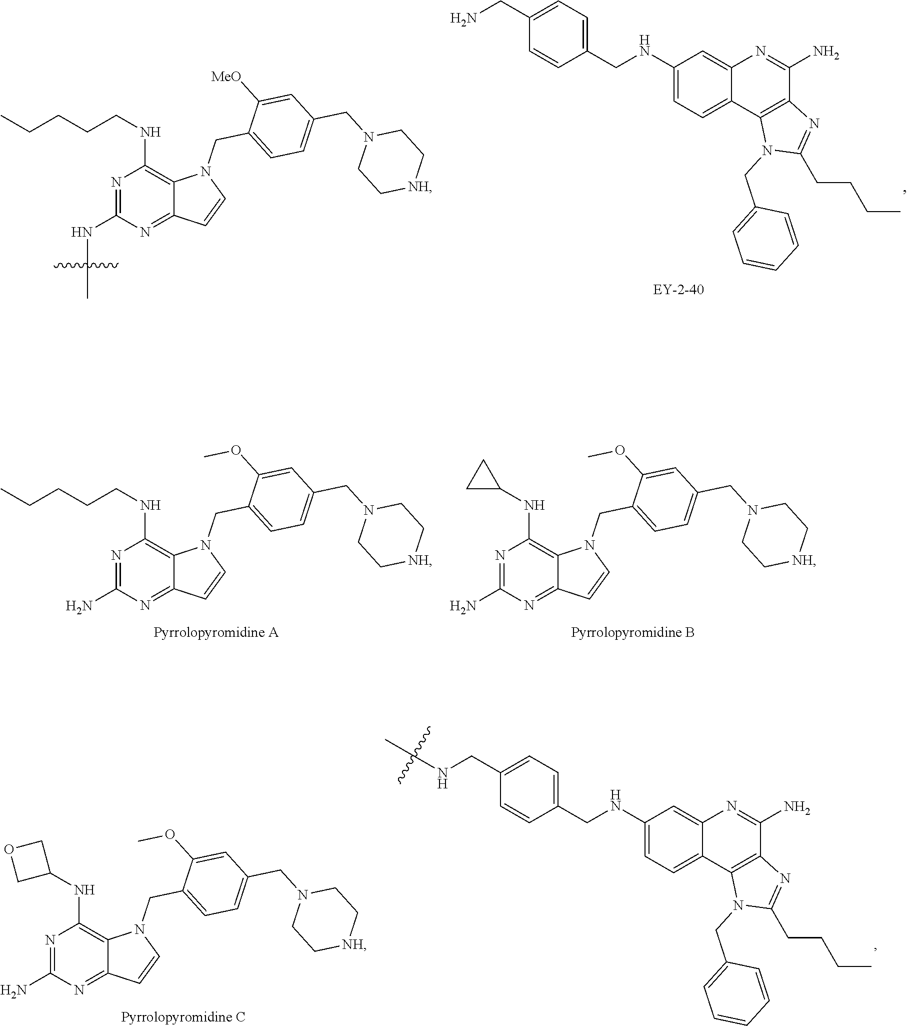 [SMILES: *Nc1nc(NCCCCC)c2c(ccn2Cc2ccc(CN3CCNCC3)cc2OC)n1 ; CCCCc1nc2c(N)nc3cc(NCc4ccc(CN)cc4)ccc3c2n1Cc1ccccc1 ; CCCCCNc1nc(N)nc2ccn(Cc3ccc(CN4CCNCC4)cc3OC)c12 ; COc1cc(CN2CCNCC2)ccc1Cn1ccc2nc(N)nc(NC3CC3)c21 ; COc1cc(CN2CCNCC2)ccc1Cn1ccc2nc(N)nc(NC3COC3)c21 ; *NCc1ccc(CNc2ccc3c(c2)nc(N)c2nc(CCCC)n(Cc4ccccc4)c23)cc1]

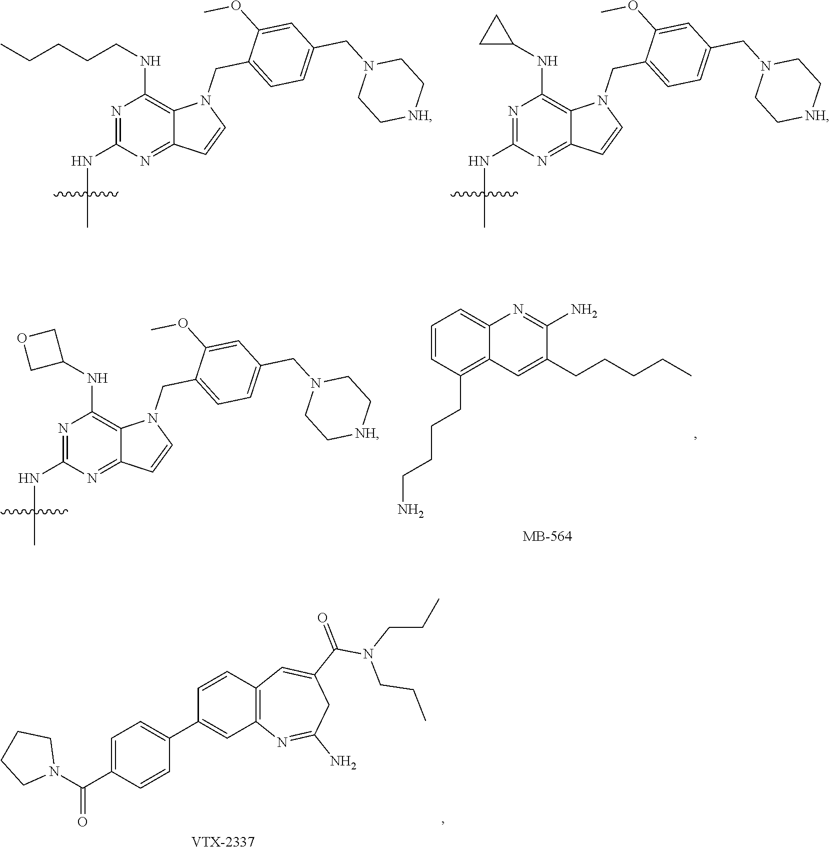

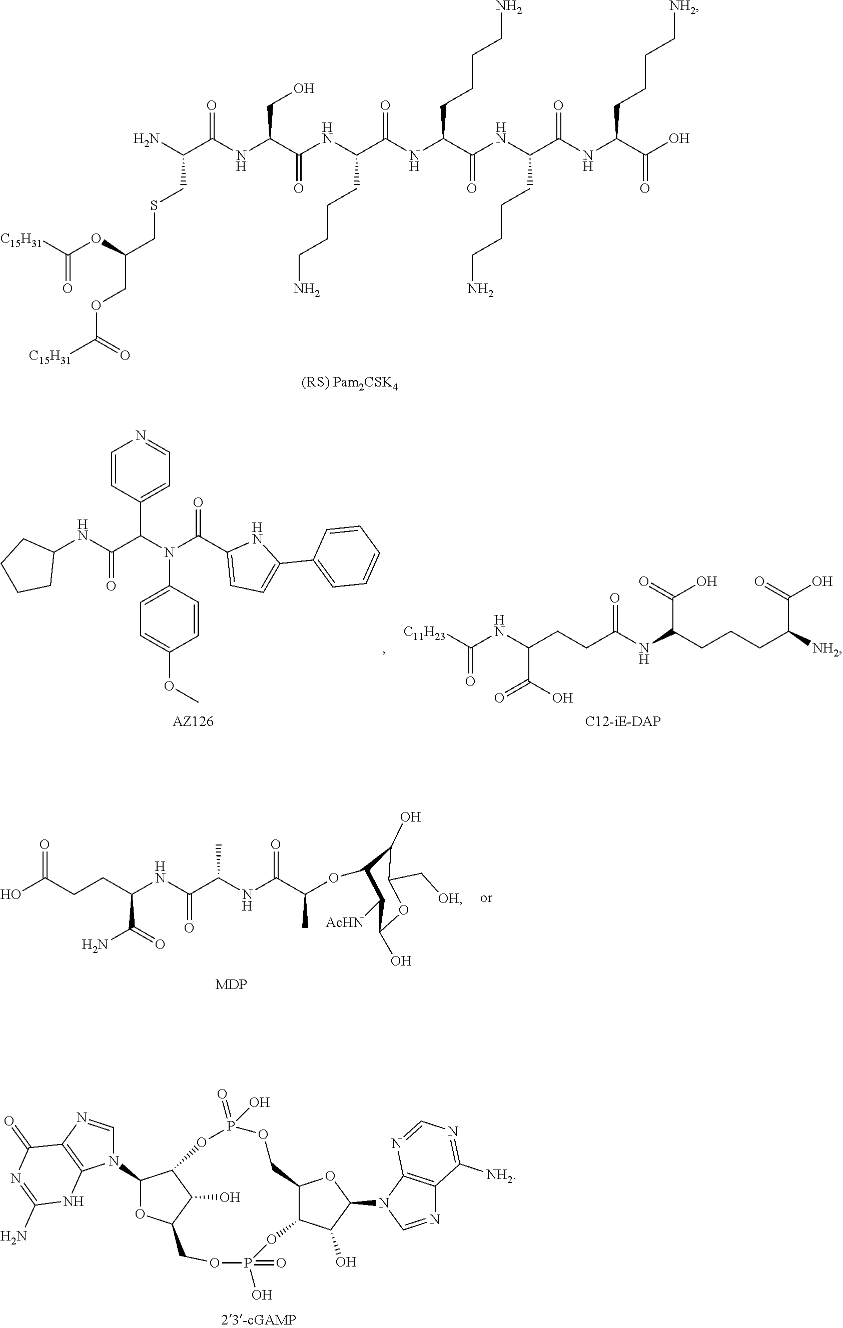

D is

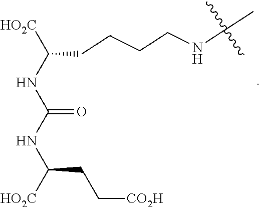 [SMILES: *NCCCC[C@H](NC(=O)N[C@@H](CCC(=O)O)C(=O)O)C(=O)O]

2. A compound of Formula I of embodiment 1,
   a diastereomer or enantiomer thereof, or a pharmaceutically acceptable salt of any of the foregoing, or a deuterated derivative or any of the forgoing, or a radioisotope of any of the forgoing,
wherein:
A is selected from the group consisting of:
¹⁷⁷Lu-DOTA, ⁹⁰Y-DOTA, ²¹²Pb-DOTA, ²²⁵Ac-DOTA, ⁴⁷Sc-DOTA, ⁶⁷Cu-DOTA, and ¹³¹I-L-Tyrosine;
B is:

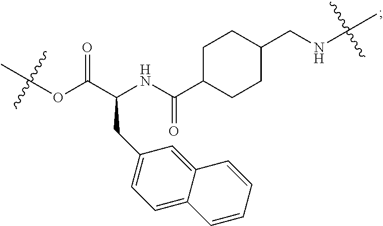 [SMILES: *NCC1CCC(C(=O)N[C@@H](Cc2ccc3ccccc3c2)C(=O)O*)CC1]

L₁ and L₂ are each independently selected from the group consisting up: (CH₂)ₙ, a dipeptide, a tripeptide, a disulfide, a hydrazone, a beta glucan,

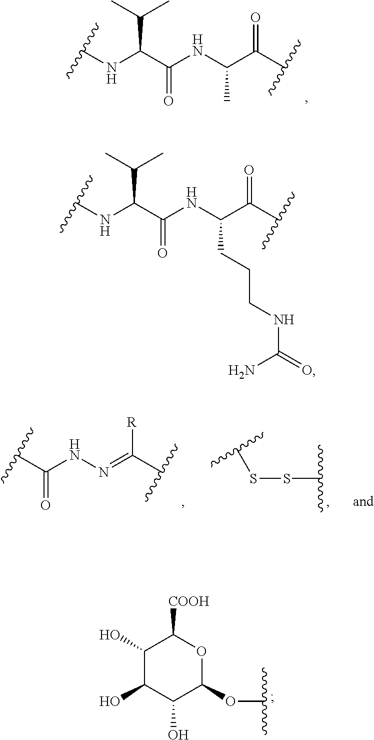

R is an aliphatic or unsaturated C1-C10, or an aromatic C6 or C10 group, wherein each R is optionally substituted with an amide, an ester, a C3-C10 cycloalkyl, an ether, or C1-C4 group;
each n is independently an integer 0-8;
C is selected from the group consisting of:

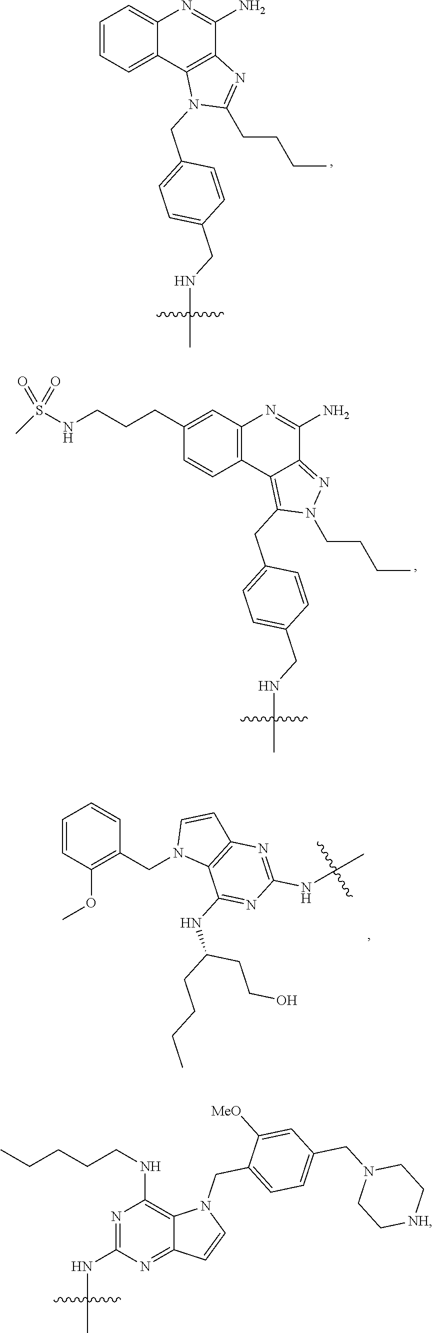

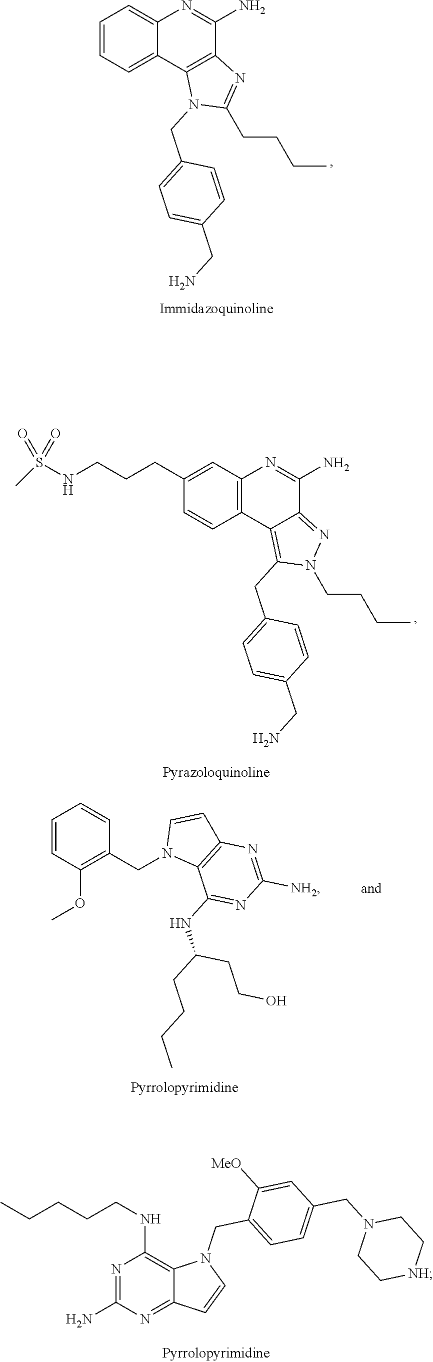

and
D is:

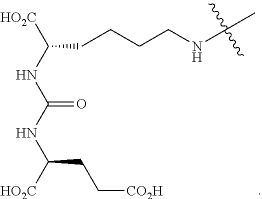 [SMILES: *NCCCC[C@H](NC(=O)N[C@@H](CCC(=O)O)C(=O)O)C(=O)O]

3. A compound of Formula I of embodiment 1,
   a diastereomer or enantiomer thereof, or a pharmaceutically acceptable salt of any of the foregoing, or a deuterated derivative or any of the forgoing, or a radioisotope of any of the forgoing, wherein
A is ¹⁷⁷Lu-DOTA;
B is:

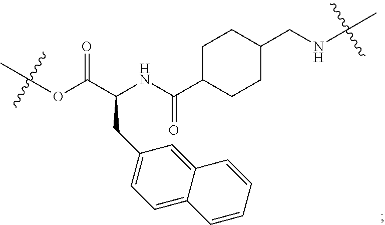 [SMILES: *NCC1CCC(C(=O)N[C@@H](Cc2ccc3ccccc3c2)C(=O)O*)CC1]

L₁ and L₂ are each independently selected from: (CH₂)ₙ, a dipeptide, a tripeptide, a disulfide, a hydrazone, a beta glucan,

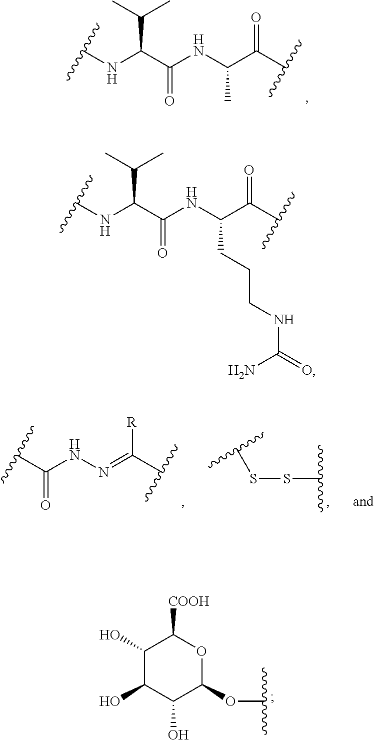

C is selected from the group consisting of:

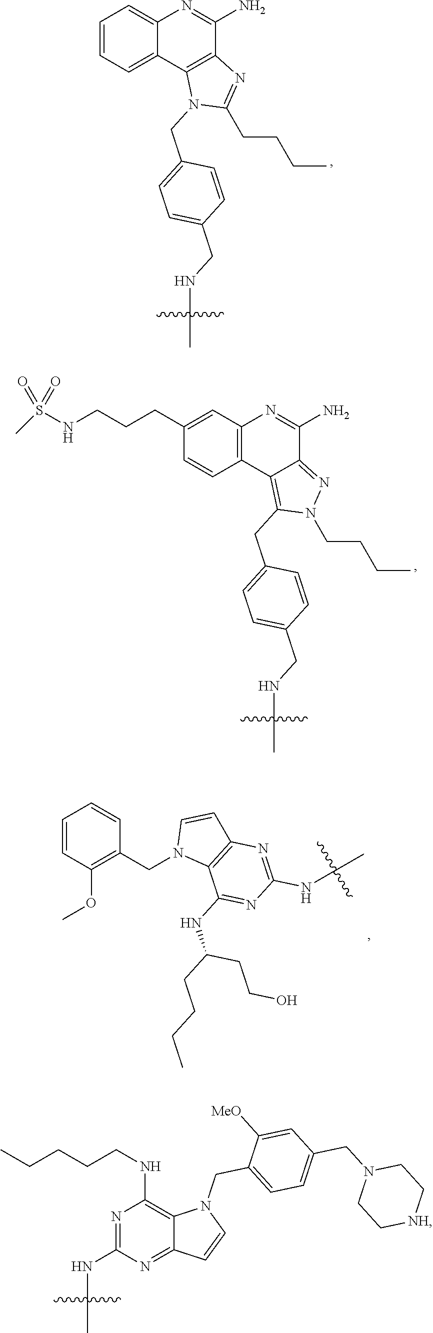

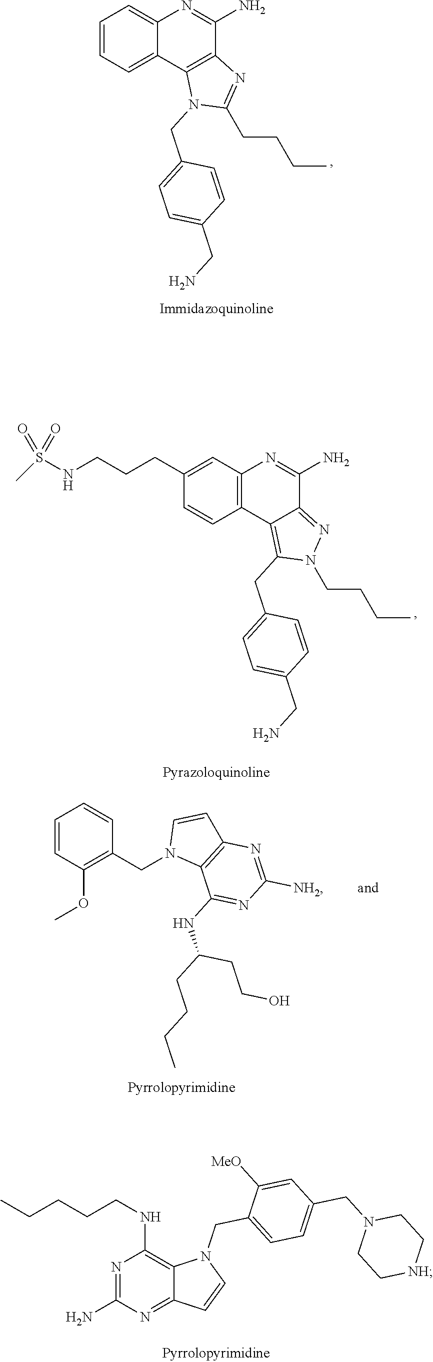

and
D is:

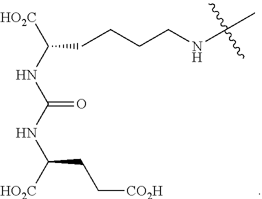 [SMILES: *NCCCC[C@H](NC(=O)N[C@@H](CCC(=O)O)C(=O)O)C(=O)O]

4. A compound of Formula 1, a diastereomer or an enantiomer of the compound, or a pharmaceutically acceptable salt of any of the foregoing, a deuterated derivative of any of the foregoing, or a radioisotope of any of the forgoing; wherein the compound of Formula I is selected from the group consisting of:

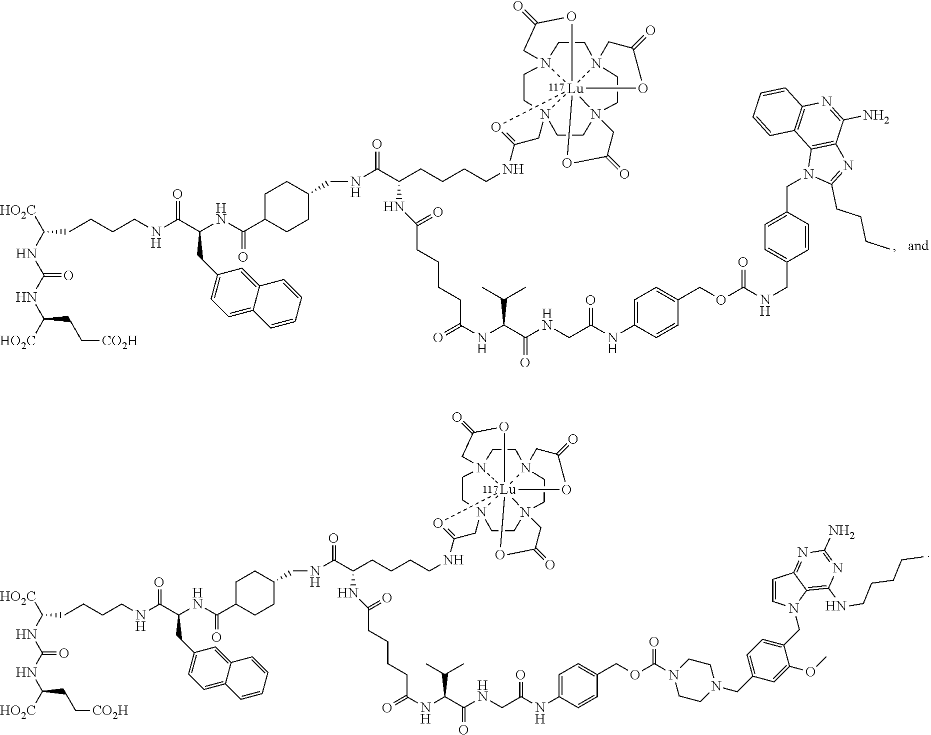

5. A composition comprising, i) the compound of Formula I, or the compound of any one of embodiments 1 to 5; the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing; and ii) an excipient, diluent, or carrier.
6. The composition of embodiment 5 that is a pharmaceutical composition.
7. The pharmaceutical composition of embodiment 6, that is in unit dose form.
8. The pharmaceutical composition of embodiment 6 or embodiment 7, further comprising an additional active agent, or a pharmaceutically acceptable salt thereof.
9. The pharmaceutical composition of any one of embodiments 6 to 8, that is in the form of a: tablet, powder, capsule, liquid, gel, emulsion, or suspension.
10. The pharmaceutical composition of any one of embodiments 6 to 9, wherein the compound of Formula I, the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing, is present in the pharmaceutical composition in an amount ranging from 0.001 mg to about 25,000 mg.
11. The pharmaceutical composition of any one of embodiments 6 to 10, wherein the compound of Formula I is a radio-immunostimulant.
12. A kit comprising the pharmaceutical composition of any one of embodiments 6 to 11, and a container.
13. The kit of embodiment 12, wherein the container is a syringe.
14. The kit of embodiment 12, wherein the container is an IV bag.
15. The kit of embodiment 12, wherein the container is a disposable container.
16. The kit of embodiment 12, wherein the container is a single use container.
17. The kit of embodiment 12, wherein the container is a resealable container.
18. A method of treating a cancer in a subject, the method comprising administering the pharmaceutical composition of any one of embodiments 6 to 11 to the subject in a therapeutically effective amount, thereby treating the cancer.
19. The method of embodiment 18, wherein the administering is selected from the group consisting of: oral, an injection; subcutaneous, intra-tumoral; systemic, local, intravenous, intraperitoneal, intramuscular, and any combination thereof.
20. A method of decreasing the size of a tumor, the method comprising contacting the tumor with the pharmaceutical composition of any one of embodiments 6 to 11 in an amount effective to decrease the size of the tumor.
21. The method of embodiment 20, wherein the tumor is comprised in a subject.
22. The method of any one of embodiments 18 to 21, wherein the subject is a subject in need thereof.
23. The method of embodiment 21 or embodiment 22, wherein the subject is a mammal.
24. The method of embodiment 22, wherein the mammal is a human.
25. The method of any one of embodiments 18 to 24, wherein the administering or the contacting is: as needed, once per day, twice per day, three times per day, once per week, once per two weeks, once per three weeks, once per month, once every six months, once per year, or for life.
26. The method of any one of embodiments 18 to 25, wherein the therapeutically effective amount ranges from about 0.001 mg to about 40,000 mg.
27. The method of any one of embodiments 18 to 26, wherein the pharmaceutical composition comprises a radio-immunostimulant (RIMS).
28. A method of administering a tumor vaccine to a subject, the method comprising administering the pharmaceutical composition of any one of embodiments 6 to 11 to the subject, thereby vaccinating the subject against the tumor.
29. The method of embodiment 28, wherein the tumor is a cancerous tumor, and wherein the subject has a cancer.
30. The method of embodiment 29, wherein the subject has a: prostate cancer, ovarian cancer, kidney cancer, colorectal cancer, NSCL cancer, castrate resistant prostate cancer.
31. The method of embodiment 30, wherein the cancer comprises the castrate resistant prostate cancer that has progressed to metastatic castrate resistant prostate cancer.
32. A method of achieving a biodistribution of a therapeutic in a tumor of a subject, the method comprising, contacting the tumor with the pharmaceutical composition of any one of embodiments 6 to 11, and wherein the pharmaceutical composition has a radioactivity measures from about 1 MBq/nmol to about 1000 MBq/nmol.
33. A method of activating an antigen presenting cell, the method comprising contacting the antigen presenting cell with the pharmaceutical composition of any one of embodiments 6 to 11.
34. A pharmaceutical composition comprising an immunostimulant, a cancer antigen targeting agent, a spacer molecule, a first linker, a second linker, and a cytotoxic agent, wherein:
   the immunostimulant is a: TLR8 agonist, TLR7 agonist, TLR2 agonist, TLR4 agonist, NOD2 agonist, NOD1 agonist, or a STING agonist, wherein
   (i) the immunostimulant is covalently bound to the first linker, and wherein the first linker is configured to release the immunostimulant when contacted with a cathepsin B mediated enzymatic cleavage;
   the second linker is covalently bound to the cytotoxic agent, the spacer molecule, or a combination thereof;
   the cytotoxic agent comprises a radiotherapeutic, a small molecule, or a combination thereof, and wherein
      the cytotoxic agent is covalently bound to the second linker, the spacer molecule, or a combination thereof; and wherein
      the cancer antigen targeting molecule targets a PSMA cell, and is covalently bound to the spacer molecule; and
   (ii) a pharmaceutically acceptable diluent, excipient, carrier.
35. The pharmaceutical composition of embodiment 34, wherein the pharmaceutical composition is a cancer vaccine in situ.
36. The pharmaceutical composition of embodiment 34 or embodiment 35, wherein the cytotoxic agent is selected from the group consisting of a: taxoid, vinca alkaloid, anthracycline, maytansinoid, tubulysin, auristatins, ¹⁷⁷Lu-H₃mpatcn, ¹⁷⁷Lu-(picaga)-DUPA, ¹⁷⁷Lu-DOTA, ⁹⁰Y-DOTA, ²²⁵Ac-DOTA, ⁴⁷Sc-DOTA, ⁴⁷Sc-(picaga)-DUPA, ⁴⁷Sc—(H₃mpatcn) ⁶⁷Cu-DOTA, and ¹³¹I-L-Tyrosine.
37. The pharmaceutical composition of embodiment 36, wherein the cytotoxic agent comprises ¹⁷⁷Lu-DOTA, ¹⁷⁷Lu-H₃mpaten, ¹⁷⁷Lu-(picaga)-DUPA.
38. The pharmaceutical composition of embodiment 37, wherein the cytotoxic agent comprises ¹⁷⁷Lu-DOTA.
39. The pharmaceutical composition of embodiment 37 or embodiment 38 wherein the cytotoxic agent comprises ¹⁷⁷Lu-(picaga)-DUPA.
40. The pharmaceutical composition of any one of embodiments 37 to 39 wherein the cytotoxic agent has a structure of Compound 1:

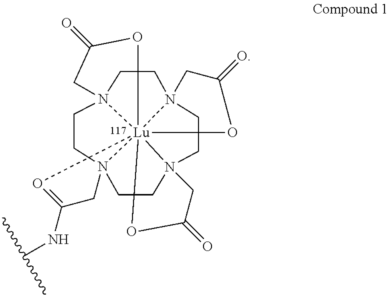

41. The pharmaceutical composition of any one of embodiments 34 to 40, wherein the cytotoxic agent is covalently bonded to a spacer molecule by an N—C bond, and wherein the first linker connects the cytotoxic agent to the spacer by a C—C bond.
42. The pharmaceutical composition of embodiment 41, wherein the spacer molecule is further covalently bonded to a cancer antigen targeting molecule, wherein the cancer antigen targeting molecule has a structure of Compound 2:

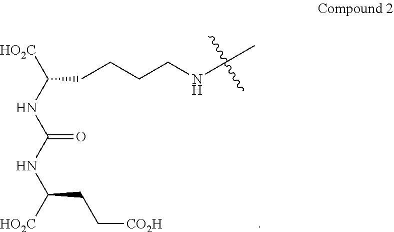 [SMILES: *NCCCC[C@H](NC(=O)N[C@@H](CCC(=O)O)C(=O)O)C(=O)O]

43. The pharmaceutical composition of any one of embodiments 34 to 42, wherein the cancer antigen targeting molecule is covalently bonded to a spacer molecule by a N—C bond.
44. The pharmaceutical composition of any one of embodiments 34 to 43, wherein the immunostimulant is covalently bound to the second linker by a C—N or C—C bond, wherein the immunostimulant is imidazoquinoline or pyrrolopyrimidine, the second linker is covalently bound to the spacer molecule by an amide or internal amine C—N bond, and the second linker is selected from the group consisting of Compound 3,

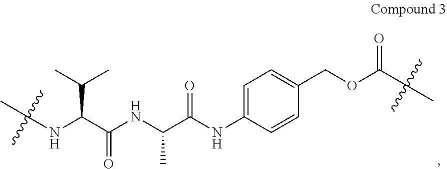 [SMILES: *N[C@H](C(=O)N[C@@H](C)C(=O)Nc1ccc(COC(*)=O)cc1)C(C)C]

a hydrazone, a disulfide, a dipeptide, beta-glucan, and a derivative or analog of any of the forgoing.
45. The pharmaceutical composition of any one of embodiments 34 to 44 wherein the immunostimulant is covalently bound to Compound 3 or a derivative or analog thereof, and the immunostimulant is selected from the group consisting of:

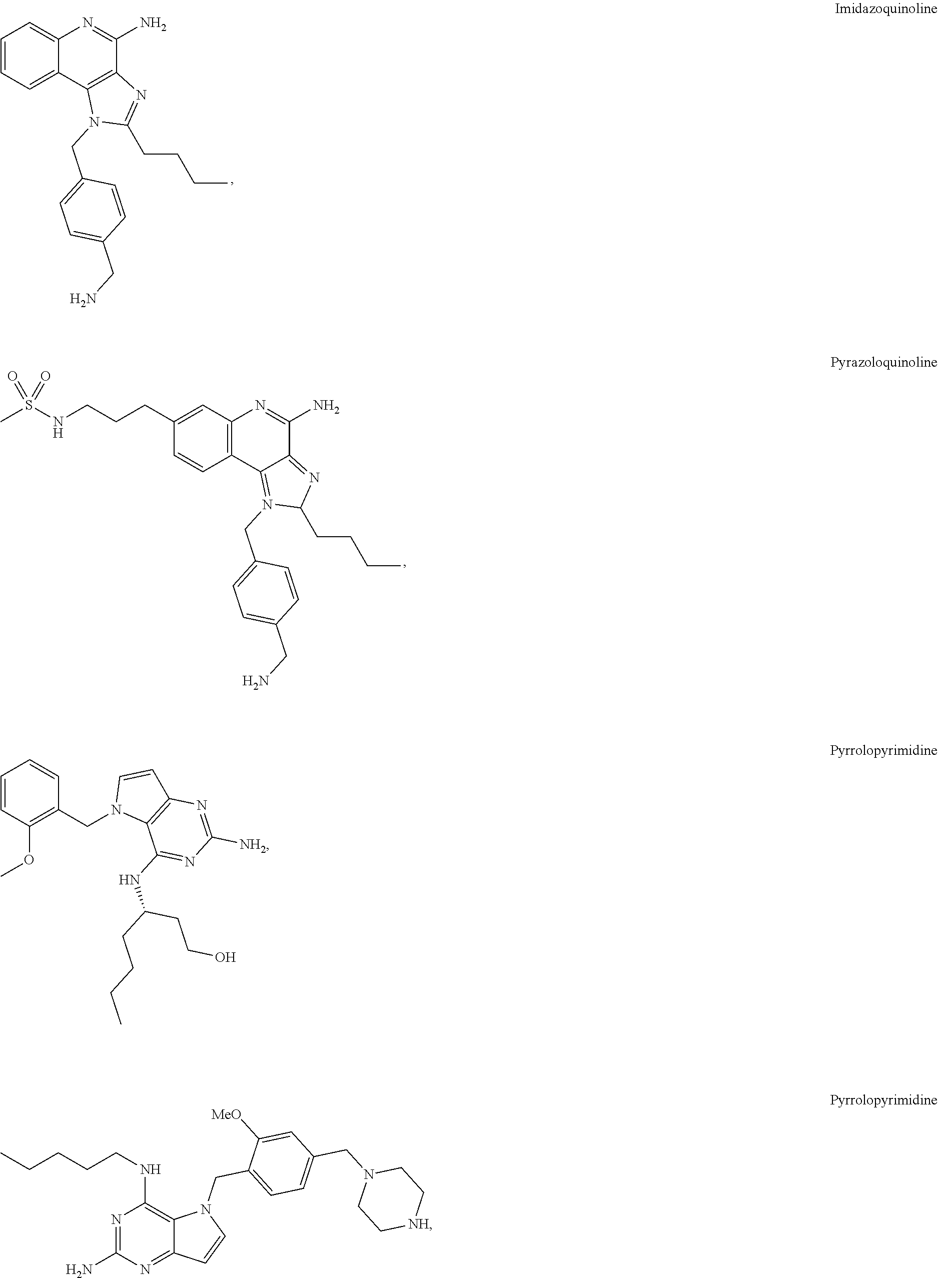

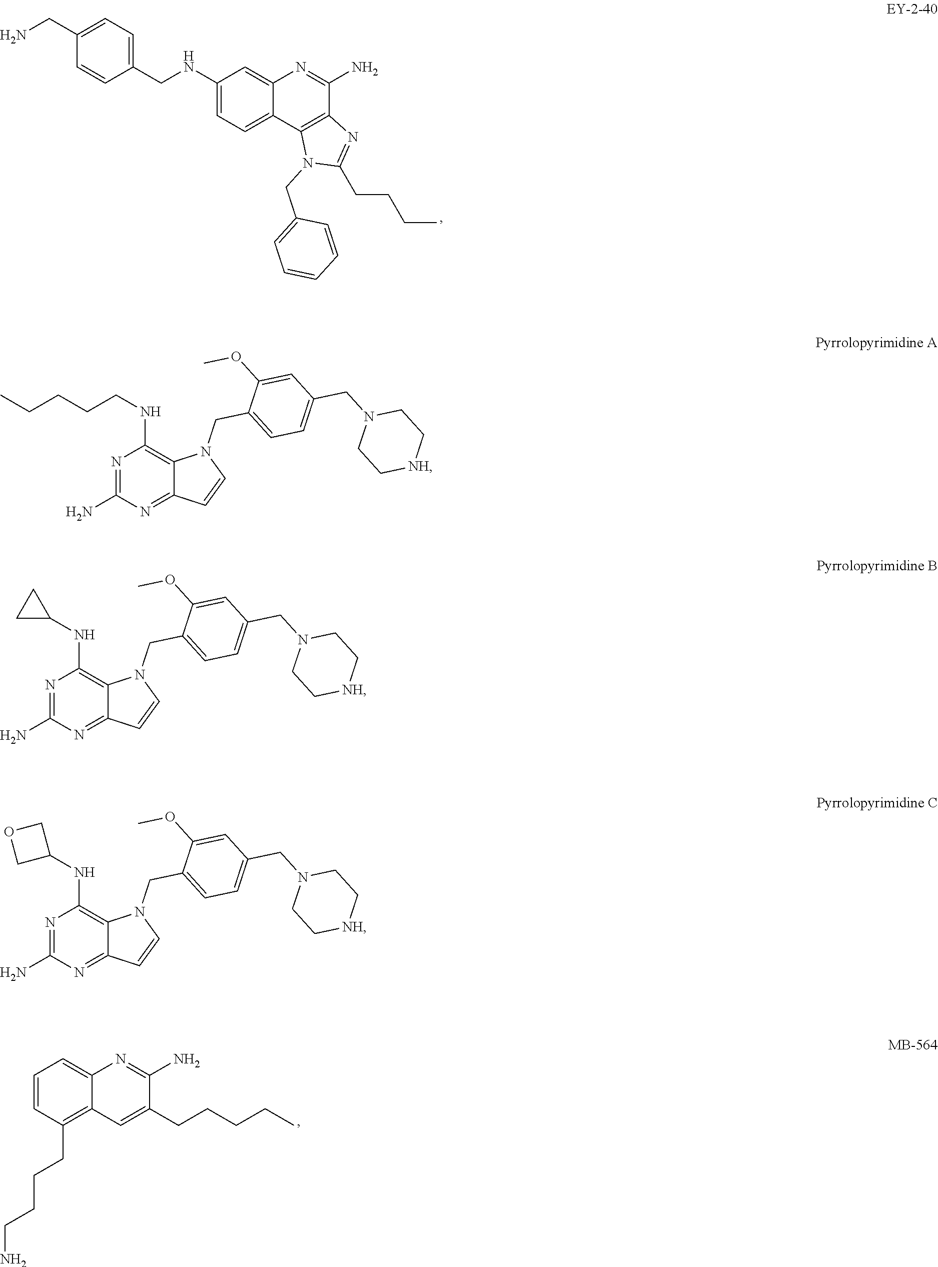

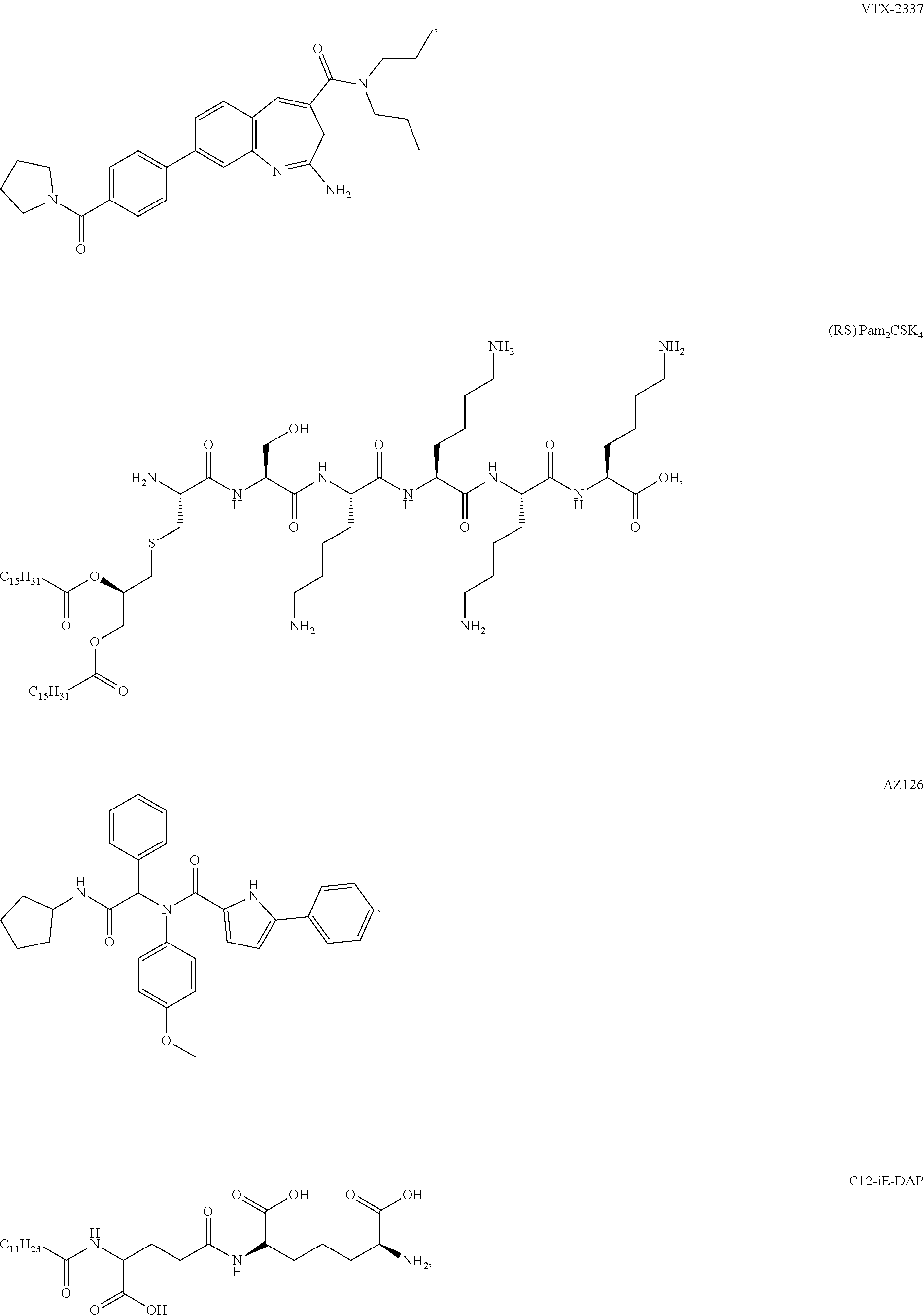

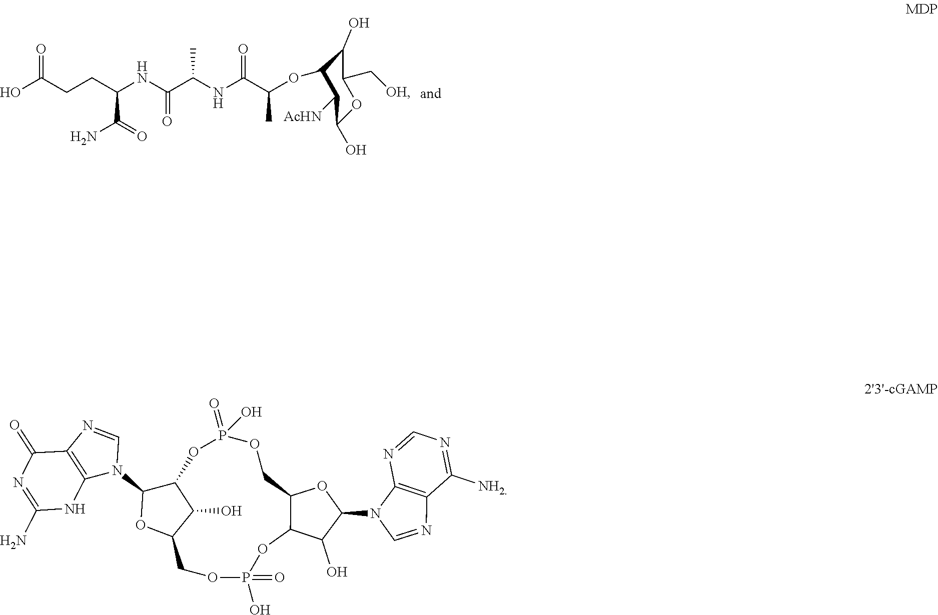

46. The pharmaceutical composition of any one of embodiments 34 to 45 wherein the spacer molecule is selected from the group consisting of:

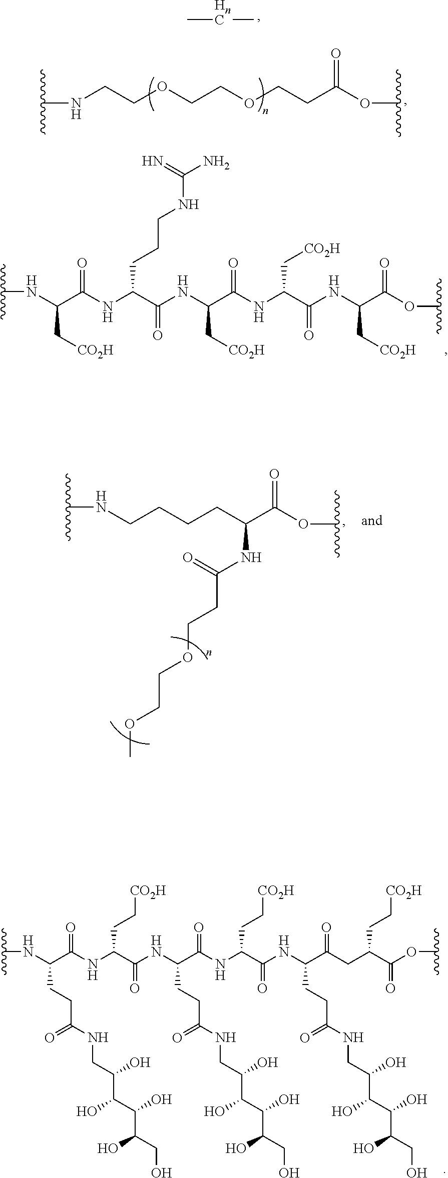

47. A composition according to the following formula:

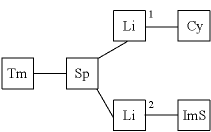

wherein:
Tm is a cancer antigen targeting molecule;
Sp is a spacer molecule coupled to the targeting molecule;
Li¹ is a first cleavable or non-cleavable linker coupled to the spacer molecule;
Li² is a second cleavable or non-cleavable linker coupled to the spacer molecule;
Cy is a cytotoxic small molecule coupled to the first cleavable or non-cleavable linker; and
ImS is a small molecule immunostimulant coupled to the second cleavable or non-cleavable linker.
48. The composition according to embodiment 47, wherein the small molecule cytotoxin is selected from the group consisting of taxoids, vinca alkaloids, anthracyclines, maytansinoids, tubulysins, auristatins, ¹⁷⁷Lu-DOTA, ⁹⁰Y-DOTA, ²²⁵Ac-DOTA, ⁴⁷Sc-DOTA, ⁶⁷Cu-DOTA, and ¹³¹I-L-Tyrosine.
49. The composition according to embodiment 47, wherein the small molecule immunostimulant is a TLR ligand selected from TLR2, TLR3, TLR4, TLR5, TLR6, TLR7, TLR7/8, TLR8, TLR9, or TLR10, a NOD-like receptor ligand, a RIG-like receptor ligand, a CLR ligand, a CDS ligand, inflammasome inducer or a STING agonist.
50. The composition according to any one of embodiments 47 to 49, wherein the first cleavable linker and the second cleavable linker are independently selected from the group consisting of:

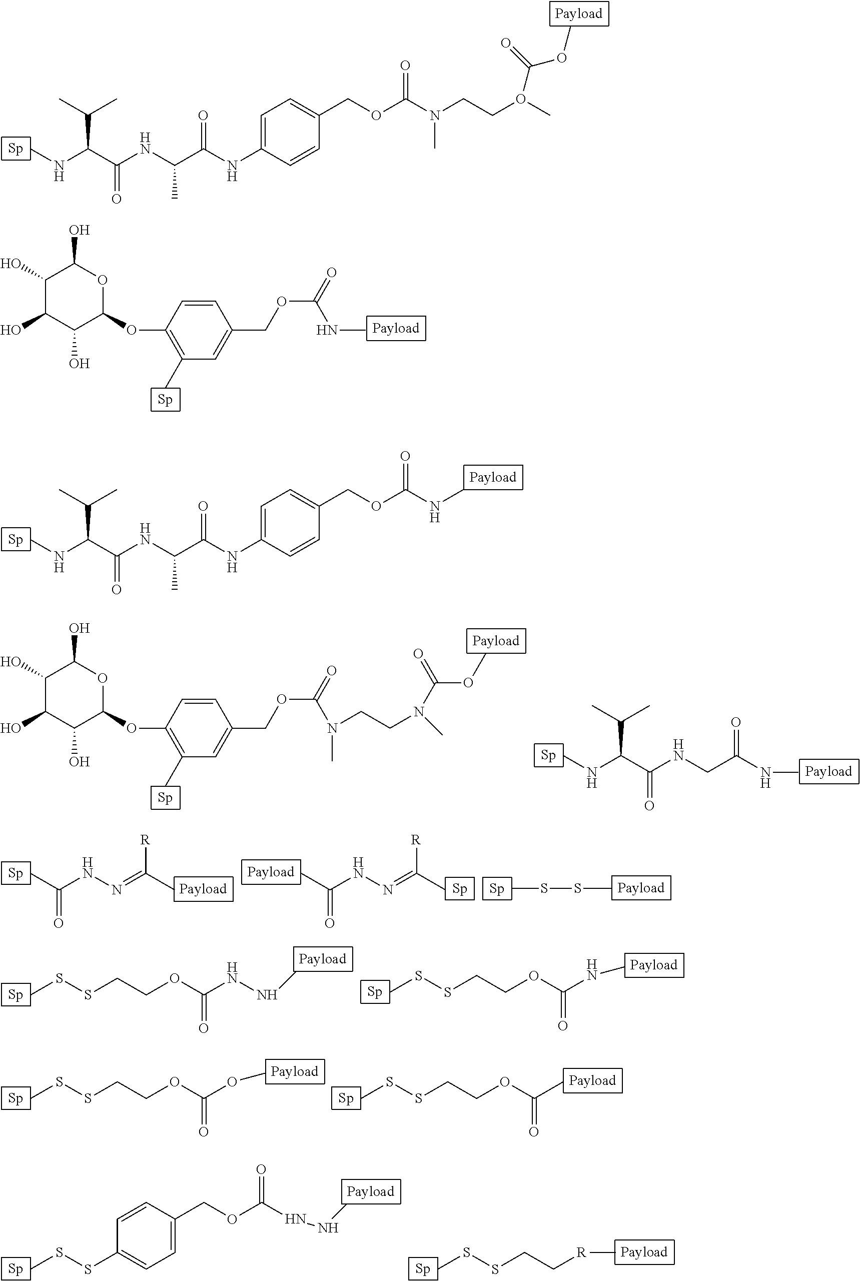

wherein Sp is the spacer molecule, and
wherein Payload is independently the cytotoxic small molecule or the small molecule immunostimulant.
51. The composition according to any one of embodiments 47 to 50, wherein the spacer molecule is selected from the group consisting of:

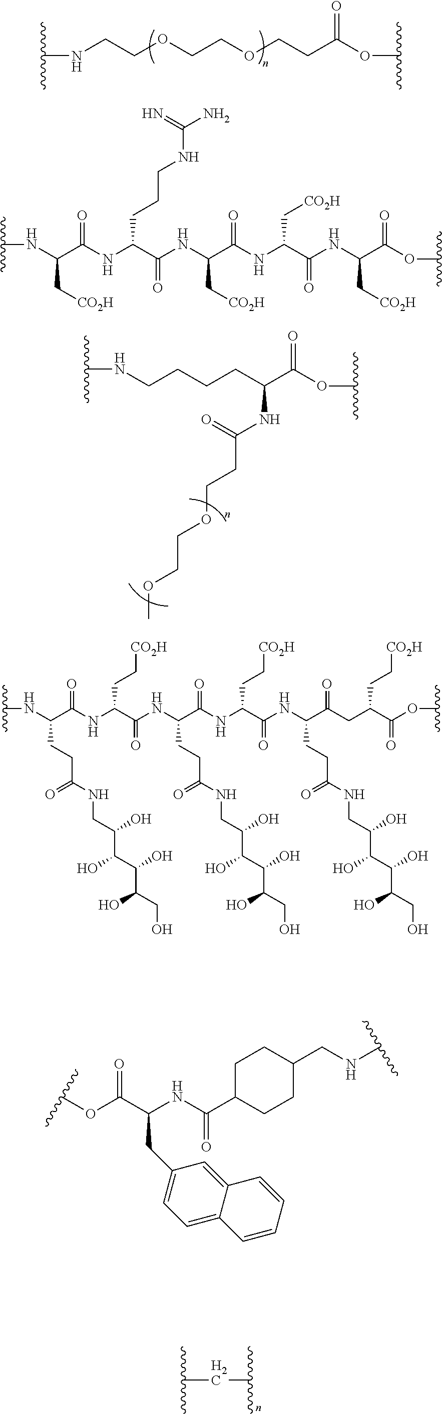

52. A composition according to the following formula:

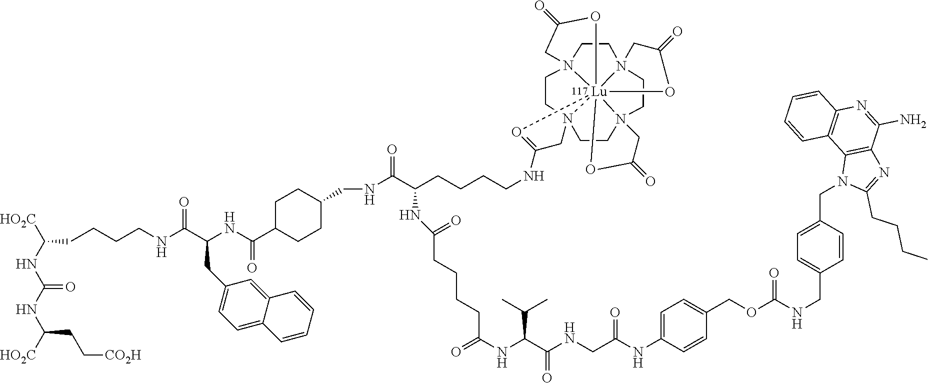

53. A method of treating cancer comprising administering to a patient in need thereof a therapeutically effective amount of the composition according to any one of embodiments 47 to 52.
54. The method of treatment according to embodiment 53, wherein the cancer comprises prostate cancer, ovarian cancer, kidney cancer, colorectal cancer or NSCL cancer.
55. The method of embodiment 53 or 54, wherein the subject is a human.
56. A pharmaceutical composition having anti-cancer activity comprising a composition of any one of embodiments 47 to 52 and a physiologically acceptable carrier.

### ADDITIONAL EMBODIMENTS

Embodiments of the disclosure described herein introduce a novel treatment paradigm that combines a small molecule cytotoxin or therapeutic radionuclide with a small molecule immunostimulant to illicit an innate and long term adaptive immune response. This will or may rescue non-responders and provide enhanced clinical outcomes in the remaining patient population.

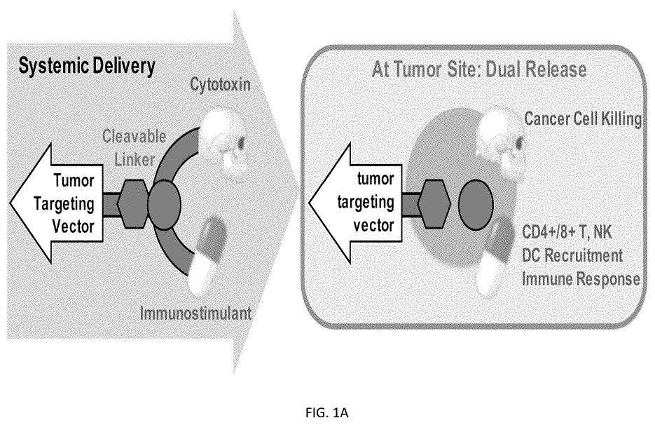

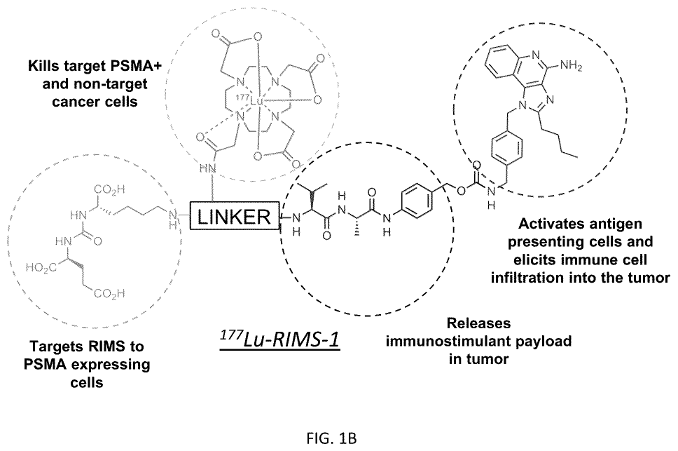

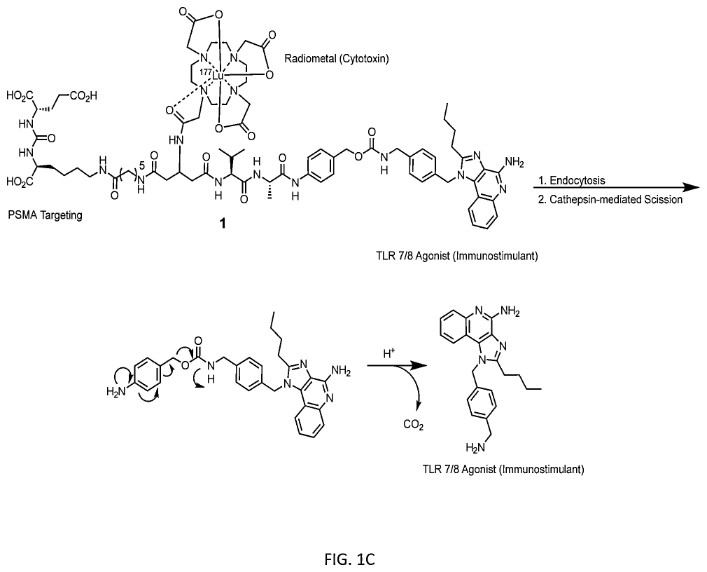

Embodiments of compositions and methods described herein provide a therapeutic platform that include: 1) A prostate specific membrane antigen targeting motif (DUPA), 2) Endosomal, enzyme cleavable linkers, chemically labile cleavable and non-cleavable linkers, 3) A cytotoxic small molecule or therapeutic radionuclide, and 4) A small molecule immunostimulant. See FIG. 1A. Specifically, the composition and method incorporates small molecule agonists of toll-like receptors (TLR's) which are important pattern recognition receptors of the innate immune system. These receptors are activated upon specific recognition of pathogen associated molecular patters (PAMP's) that are distinct based on the pathogen, eliciting an immune response. The natural ligands for TLR7 and TLR8 are single-stranded RNA; these endosomal TLRs can also be activated by synthetic small molecule TLR 7/8 agonists. These immunostimulants directly activate antigen presenting cells (APC's) such as monocytes, macrophages and dendritic cells, inducing the NF-κB mediated transcription of cytokines and chemokines resulting in enhanced humoral and cellular immune response, and Th1-biased responses, providing intense phagocytic activity. The composition and method incorporates a potent dual TLR 7/8 small molecule agonist of the imidazoquinoline class of compounds with low nM activity. Briefly, the localization/activation of immune cells and increased phagocytic activity catalyzed by the TLR 7/8 agonist combined with the apoptotic cancer cell debris generated by the cytotoxic payload, results in tumor antigen loaded APC's (eg. dendritic cells). This generates recruitment of tumor specific cytotoxic T-cells that kills cancer cells expressing the tumor associated antigens. See FIG. 2. This adaptive immune response provides improved clinical outcomes. Importantly, this approach shows efficacy against heterogeneous PSMA(+/−) populations due to: 1) Non-discriminatory phagocytosis of cancer cells via the localized immune cell population (e.g. macrophages) stimulated by the TLR 7/8 agonist, 2) The bystander effect, which results in some PSMA(−) cell killing, with the resultant antigen debris undergoing phagocytosis by infiltrating dendritic cells, activating between 100-3000 killer T-cells, which are then primed to target PSMA(−) cancer cells systemically. This promotes targeted, distal tumor cell death against PSMA(−) cells that have detached from the original heterogenous tumor population and prevents a major resistance pathway in prostate cancer. The composition and method also can solve a challenge in small molecule immunostimulant therapy, specifically, the difficulty of systemic administration due to cytokine storm related toxicities. This is highlighted in recently published work that combines external radiation with systemic TLR 7/8 agonist administration resulting in substantial tumor regression but notable cytokine responses

FIG. 2A illustrates a targeted immunostimulant delivery mechanism of action: i) Binding of tumor targeting vector to surface tumor antigen, ii) Endocytosis, followed by release of cytotoxin and immunostimulant, iii) Cancer cell death, iv) Stimulation of TLR7/8 receptors on nearby immune cells, stimulating an immune response, v) Localization of immune cells to remaining target and non-target cancer cells at tumor and vi) Eradication of remaining cancer cells. Embodiments of the disclosure include conjugates that incorporate a diversity of cytotoxins with varying mechanisms of action, including but not limited to tubulin polymerization inhibitors, tubulin polymerization stabilizers, DNA intercalators, DNA alkylators and the cytotoxic radionuclides ¹⁷⁷Lu/⁹⁰Y/²²⁵Ac/⁴⁷Sc/⁶⁷Cu/¹³¹I. Selection of cytotoxin is based on clinical applicability to facilitate translation for future human clinical trials. Embodiments of the disclosure incorporate cytotoxins that display immunomodulatory effects that can synergize with immunotherapies, leading to enhanced efficacy due to long-term immune memory response. The linker length and functionality is varied based on linker motifs that have high PSMA affinity, good in vivo stability and pharmacokinetics. The resultant analogues were screened for binding affinity to PSMA, plasma stability and incubated with cathepsin B enzyme to ensure release of the TLR 7/8 payload. The compositions are then tested against PSMA(+) cells (LNCaP, MDA PCa 2b, CWR22Rv1) to determine the IC50's and against PSMA(−) cells (PC3) as a negative control. These studies were performed with and without the presence of isolated hPBMC's to observe any augmented cell killing. The supernatant from the former study was analyzed for IFN-α, chemokines and cytokines levels using ELISA and magnetic bead-based multiplexed assay kits.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: Compound Synthesis, Purification, and Characterization

Compounds herein and intermediates used in the preparation of compounds herein can be prepared using procedures shown in the following examples and related procedures. The methods and conditions used in these examples, and the actual compounds prepared in these examples, are not meant to be limiting, but are meant to demonstrate how the compounds of the current disclosure can be prepared. Starting materials and reagents used in these examples, when not prepared by a procedure described herein, are generally either commercially available, or are reported in the chemical literature, or may be prepared by using procedures described in the chemical literature. Column chromatography was performed with pre-packed silica gel cartridges or manually loaded column chromatography systems. Preparative high performance liquid chromatography (HPLC) was performed using a reverse phase column as indicated of a size appropriate to the quantity of material being separated, generally eluting with a gradient of increasing concentration of methanol or acetonitrile in water, also containing 0.05% or 0.1% trifluoroacetic acid or 10 mM ammonium acetate, at a rate of elution suitable to the column size and separation to be achieved.

RIMS compounds were synthesized to generate a compound with the moieties shown in FIG. 1B by conventional synthetic methods. Various linkers are included in the final RIMS compounds.

### Example 1.1: Synthesis of RIMS Compound 1

The synthesis of compound 5 as shown in Scheme 1 was carried out on a 0.16 mmol scale using 1 on Wang resin (200 mg, 0.8 mmol/g). The resin was swollen in DCM and DMF followed by treatment with a mixture of 20% piperidine in DMF. After 30 min the resin was washed with DMF and DCM. Next, 1 eq. of triphosgene was added in the presence of base under N2 atmosphere to form the isocyanate derivate in situ. After 30 min, 4 (250 mg, 0.64 mmol) and DIEA (112 μL, 0.64 mmol) were added and the reaction was allowed to stir at room temperature. After 12 h the resin was washed with DMF, DCM, Et2O and dried under vacuum. To confirm urea bond formation, a small aliquot of resin was treated with mixture of 1% TFA in DCM to selectively remove the Mtt (4-methyl trityl) protecting group (since the Mtt-free intermediate is easier to detect on LCMS). Finally, after washing and drying the Glu-urea-Lys was cleaved from the resin by treatment with a mixture of TFA/TIS/H2O (95%/2.5%/2.5%). After cleavage from the resin the compound was lyophilized. The crude product was purified by reverse-phase semi-preparative HPLC and characterized by analytical HPLC and MS measurements. Yield: (0.027 g, 39%). 1H NMR of 5′ (500 MHZ, CD3OD) δ 4.31 (quin, J=5.0 Hz, 2H), 2.92 (m, 2H), 2.42 (m, 2H), 2.16 (m, 1H), 1.90 (m, 2H), 1.68 (m, 3H), 1.50 (m, 2H). HRESIMS [M+H]+ calcd for C₁₂H₂₁N₃O₇ 320.1458, found 320.1451.

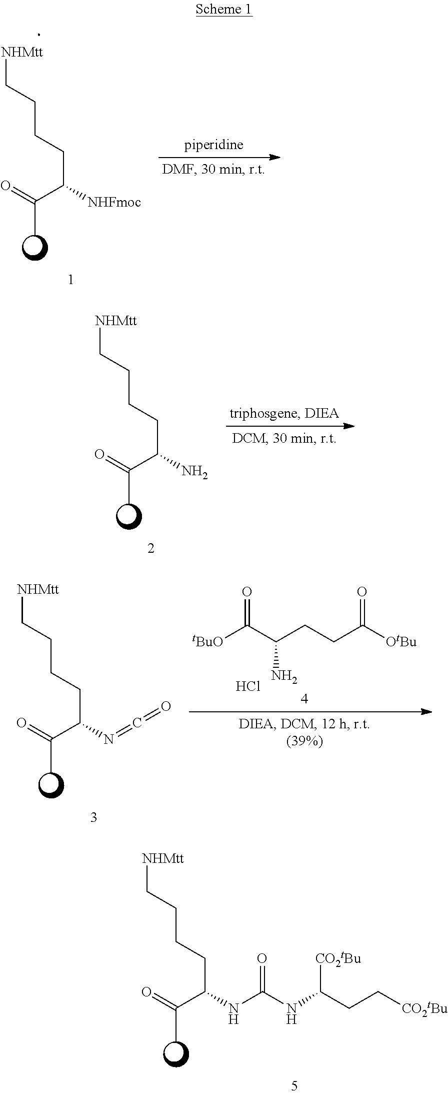

The synthesis of compound 7 as shown in Scheme 2 was synthesized starting from compound 5 on Wang resin (200 mg, 0.16 mmol) was treated with mixture of 1% TFA in DCM to selectively remove the Mtt protecting group. Acid 6 (280 mg, 0.64 mmol) was coupled to resin using PyBOP (111 mg, 0.22 mmol) in the presence of DIEA (112 μL, 0.64 mmol) for 12 h. After coupling, a small aliquot of resin was treated with a mixture of 20% piperidine in DMF in order to remove Fmoc group (since the Fmoc-free intermediate is easier to detect on LCMS). Finally, after washing and drying the Glu-urea-Lys-nap was cleaved from the resin by treatment with a mixture of TFA/TIS/H2O (95%/2.5%/2.5%). After cleavage from the resin the compound was lyophilized. The crude product was purified by reverse-phase semi-preparative HPLC and characterized by analytical HPLC and MS measurements. Yield: (0.021 g, 38%). 1H NMR of 7′ (500 MHz, CD3OD) δ 7.88 (d, J=10 Hz, 1H), 7.86 (m, 2H), 7.74 (s, 1H), 7.49 (m, 2H), 7.41 (m, 1H), 4.31 (dd, J=8.5, 3.5 Hz, 1H), 4.15 (dd, J=8.7, 3.8 Hz, 1H), 4.10 (t, J=7.5 Hz, 1H), 3.29 (m, 1H), 3.25 (m, 1H), 3.19 (m, 1H), 3.05 (m, 1H), 2.41 (m, 2H), 2.15 (m, 1H), 1.90 (m, 1H), 1.64 (m, 1H), 1.48 (m, 1H), 1.29 (m, 2H), 1.22 (m, 2H).

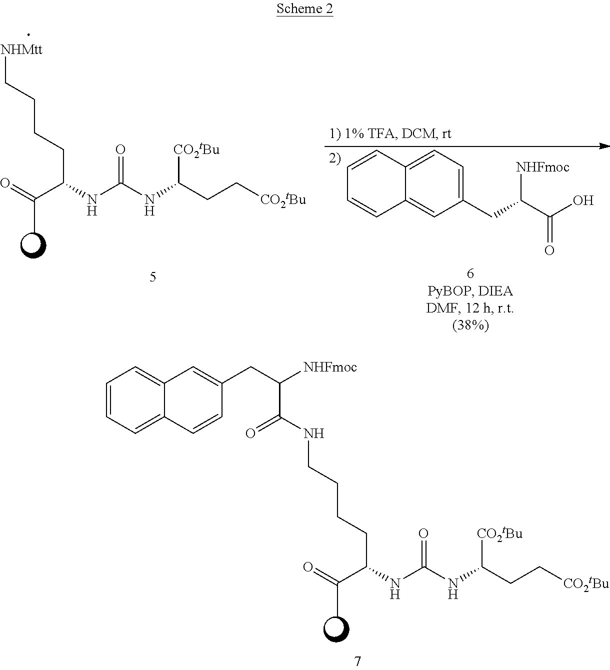

The synthesis of compound 9 as shown in Scheme 3 was synthesized starting from compound 7 on Wang resin (200 mg, 0.16 mmol) was treated with mixture of 20% piperidine in DMF. After washing, acid 8 (243 mg, 0.64 mmol) was coupled using PyBOP (167 mg, 0.32 mmol) in the presence of DIEA (112 μL, 0.64 mmol) for 12 h. After coupling, a small aliquot of resin was treated with mixture of 20% piperidine in DMF in order to remove Fmoc group (since the Fmoc-free intermediate is easier to detect on LCMS). After washing and drying the peptide was cleaved from the resin by treatment with a mixture of TFA/TIS/H2O (95%/2.5%/2.5%). After cleavage from the resin the compound was lyophilized. The crude product was purified by reverse-phase semipreparative HPLC and characterized by analytical HPLC and MS measurements. Yield: (0.019 g, 44%). 1H NMR of 9′ (500 MHZ, CD3OD) δ 7.79 (m, 3H), 7.68 (s, 1H), 7.43 (m, 2H), 7.40 (m, 1H), 4.67 (dd, J=8.7, 2.1 Hz, 1H), 4.31 (dd, J=8.7, 3.6 Hz, 1H), 4.18 (dd, J=8.5, 3.8 Hz, 1H), 3.23 (dd, J=13.7, 6.5 Hz, 1H), 3.13 (m, 2H), 3.07 (dd, J=11.0, 5.0 Hz, 1H), 2.75 (d, J=7.0 Hz, 2H), 2.41 (m, 2H), 2.16 (m, 2H), 1.87 (m, 3H), 1.77 (m, 1H), 1.68 (m, 1H), 1.63 (m, 1H), 1.54 (m, 2H), 1.44 (m, 1H), 1.37 (m, 2H), 1.29 (m, 3H), 1.01 (m, 2H). HRESIMS [M+H]+ calcd for C₃₃H₄₆N₅O₉ 656.3296, found 656.3291.

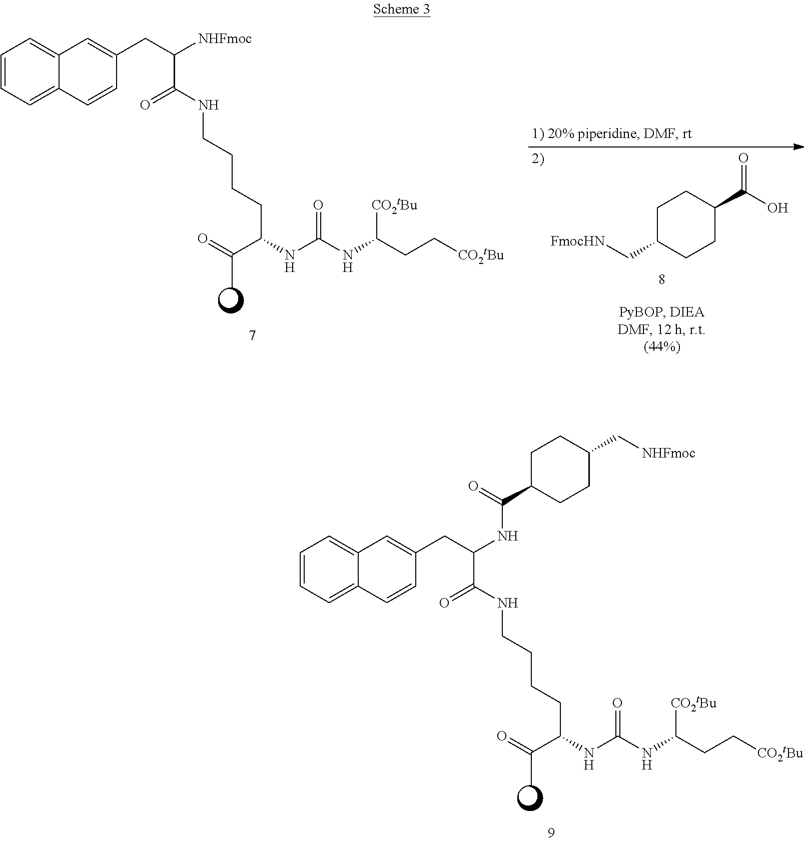

The synthesis of compound 11 as shown in Scheme 4 was synthesized starting from compound 9 on Wang resin (200 mg, 0.16 mmol) was treated with a mixture of 20% piperidine in DMF. After washing, acid 10 (400 mg, 0.64 mmol) was coupled to the resin using PyBOP (167 mg, 0.32 mmol) in the presence of DIEA (112 μL, 0.64 mmol) for 12 h. After coupling, a small aliquot of resin was treated with mixture of 20% piperidine in DMF in order to remove Fmoc group and subsequently this resin was treated with mixture of 1% TFA in DCM to selectively remove Mtt protecting group (since the Mtt/Fmoc-free intermediate is easier to detect on LCMS). Finally, after washing and drying the peptide was cleaved from the resin by treatment with a mixture of TFA/TIS/H2O (95%/2.5%/2.5%). After cleavage from the resin the compound was lyophilized. The crude product was purified by reverse-phase semi-preparative HPLC and characterized by analytical HPLC and MS measurements. Yield: (0.015 g, 42%). 1H NMR of 11′ (500 MHz, CD3OD) δ 7.79 (m, 3H), 7.68 (s, 1H), 7.43 (m, 2H), 7.39 (dd, J=8.4, 1.8 Hz, 1H), 4.65 (dd, J=8.5, 6.6 Hz, 1H), 4.32 (dd, J=8.5, 5.0 Hz, 1H), 4.17 (dd, J=8.7, 4.7 Hz, 1H), 3.81 (t, J=6.4 Hz, 1H), 3.24 (dd, J=13.7, 6.5 Hz, 1H), 3.17-3.00 (m, 5H), 2.92 (m, 2H), 2.41 (m, 2H), 2.14 (m, 2H), 1.86 (m, 5H), 1.69 (m, 4H), 1.62 (m, 1H), 1.54 (m, 1H), 1.44 (m, 3H), 1.36 (m, 3H), 1.27 (m, 3H), 0.95 (m, 2H). HRESIMS [M+H]+ calcd for C₃₉H₅₈N₇O₁₀ 784.4245, found 784.4237.

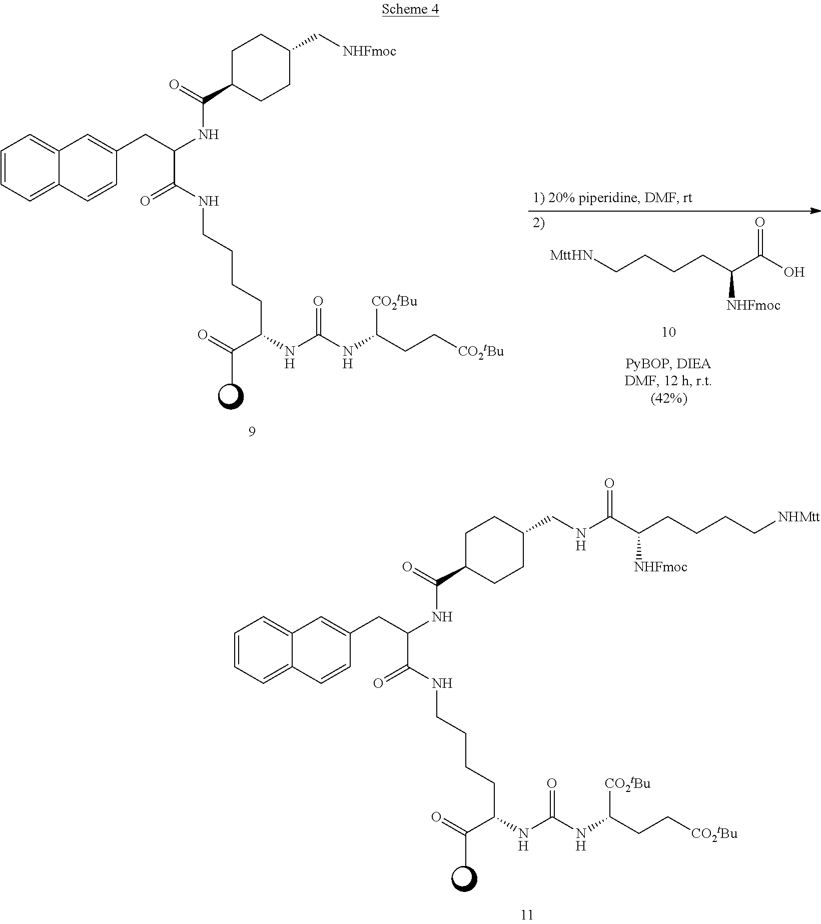

The synthesis of compound 13 as shown in Scheme 5 was synthesized starting from compound 11 on Wang resin (200 mg, 0.16 mmol) was treated with a mixture of 1% TFA in DCM. After washing, acid 12 (261 mg, 0.32 mmol) was coupled to the resin using PyBOP (167 mg, 0.32 mmol) in the presence of DIEA (112 μL, 0.64 mmol) for 16 h. After coupling and washing, the small aliquot of resin-bounded peptide was treated with mixture of 20% piperidine in DMF in order to remove Fmoc group (since the Fmoc-free intermediate is easier to detect on LCMS). Next, this aliquot of peptide was washed, dried and cleaved from the resin by treatment with a mixture of TFA/TIS/H2O (95%/2.5%/2.5%). After cleavage from the resin, the compound was lyophilized. The crude product was purified by reverse-phase semi-preparative HPLC and characterized by analytical HPLC and MS measurements. Yield: (0.023 g, 55%). 1H NMR of 13′ (500 MHz, CD3OD) δ 7.80 (m, 3H), 7.68 (s, 1H), 7.44 (m, 2H), 7.40 (dd, J=8.5, 1.8 Hz, 1H), 4.65 (dd, J=8.7, 6.5 Hz, 1H), 4.31 (dd, J=8.5, 5.0 Hz, 1H), 4.17 (dd, J=8.5, 4.7 Hz, 1H), 3.87 (t, J=6.4 Hz, 1H), 3.81 (bs, 6H), 3.29 (m, 16H), 3.23 (m, 5H), 3.17-3.00 (m, 5H), 3.00 (dd, J=13.4, 7.3 Hz, 1H), 2.41 (m, 2H), 2.14 (m, 2H), 1.86 (m, 5H), 1.70 (m, 2H), 1.62 (m, 1H), 1.55 (m, 3H), 1.38 (m, 5H), 1.29 (m, 3H), 0.96 (m, 2H). HRESIMS [M+H]+ calcd for C₅₅H₈₄N₁₁O₁₇ 1170.6047, found 1170.6036.

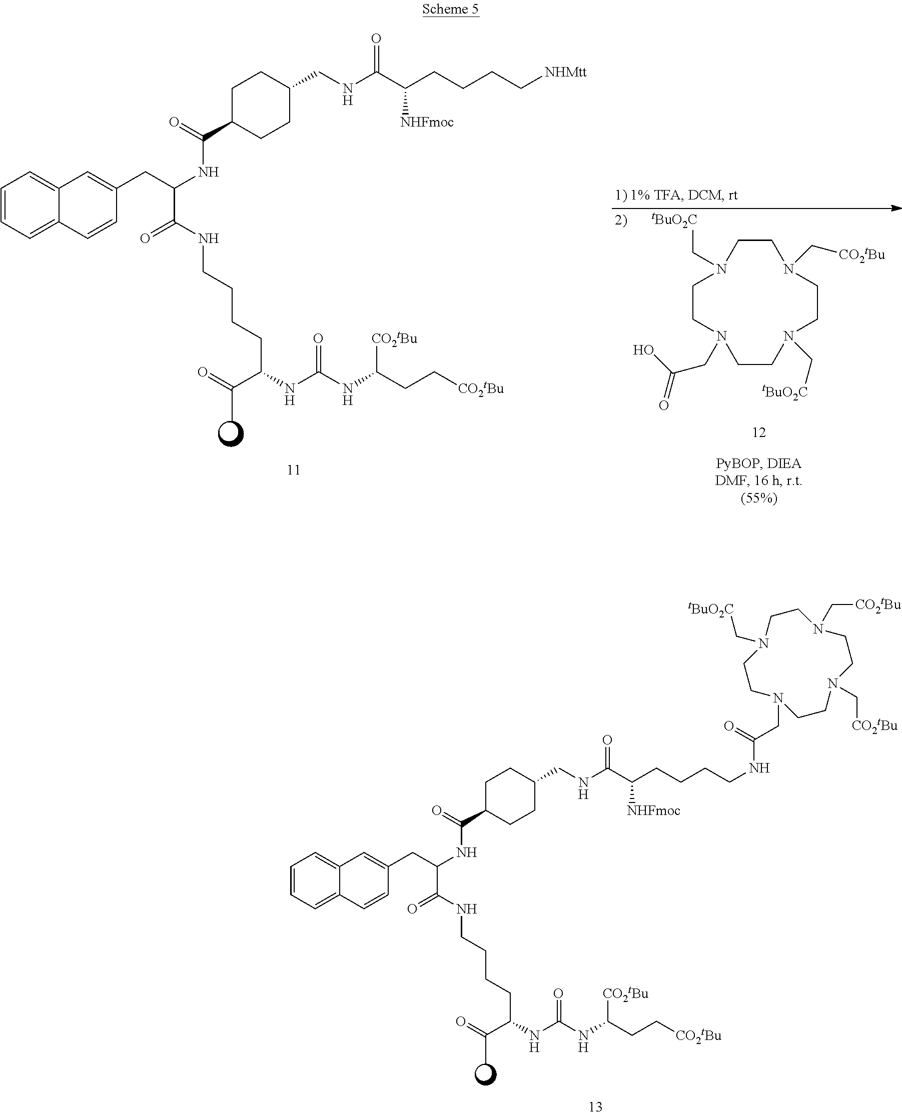

The synthesis of compound 15 as shown in Scheme 6 was synthesized starting from compound 13 on Wang resin (200 mg, 0.16 mmol) was treated with mixture of 20% piperidine in DMF in order to remove Fmoc group. After washing, acid 14 (94 mg, 0.64 mmol) was directly coupled using PyBOP (167 mg, 0.32 mmol) as a coupling reagent in the presence of DIEA (112 μL, 0.64 mmol) for 24 h. After coupling and washing, a small aliquot of resin-bounded peptide was cleaved from the resin by treatment with a mixture of TFA/TIS/H₂O (95%/2.5%/2.5%). After cleavage from the resin, the compound was lyophilized. The crude product was purified by reverse-phase semipreparative HPLC and characterized by analytical HPLC and MS measurements. Yield: (0.023 g, 55%). 1H NMR of 15′ (500 MHZ, CD3OD) δ 7.79 (m, 3H), 7.68 (s, 1H), 7.44 (m, 2H), 7.39 (dd, J=8.5, 1.8 Hz, 1H), 4.65 (dd, J=8.5, 6.5 Hz, 1H), 4.32 (dd, J=8.7, 5.0 Hz, 1H), 4.28 (dd, J=8.7, 5.8 Hz, 1H), 4.18 (dd, J=8.55, 4.7 Hz, 1H), 3.88 (bs, 6H), 3.29 (m, 16H), 3.24 (m, 5H), 3.16-2.94 (m, 6H), 2.41 (m, 2H), 2.31 (t, J=5.9 Hz, 2H), 2.28 (t, J=7.2 Hz, 2H), 2.14 (m, 2H), 1.89 (m, 1H), 1.78 (m, 3H), 1.70 (m, 2H), 1.62 (m, 5H), 1.54 (m, 3H), 1.37 (m, 6H), 1.26 (m, 3H), 0.92 (m, 2H). HRESIMS [M+H]+ calcd for C₆₁H₉₂N₁₁O₂₀ 1298.6520, found 1298.6514.

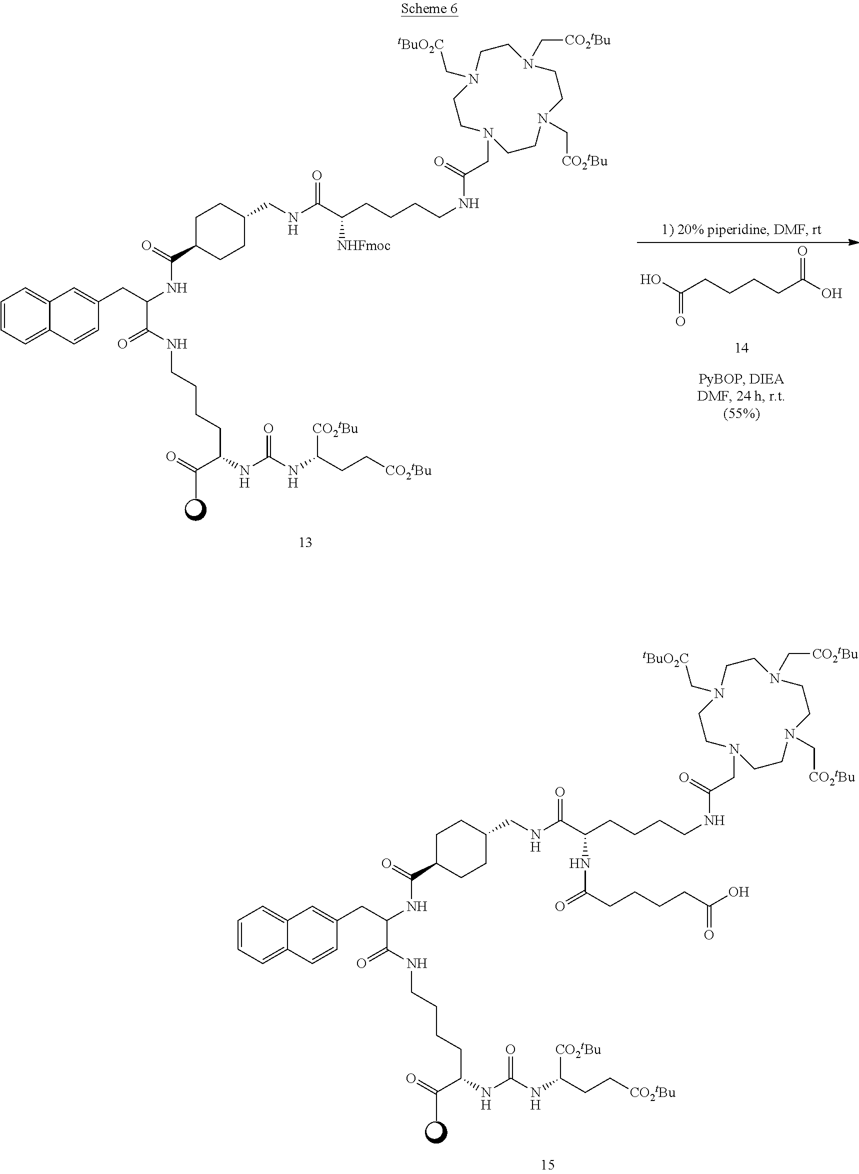

The synthesis of compound 17 as shown in Scheme 7 was synthesized starting from compound 15 on Wang resin (200 mg, 0.16 mmol) was washed with DMF and added to the solution of EDC*HCl (1.534 g, 8.00 mmol) in DMF in the presence of DIEA (1.393 mL, 8.00 mmol). After 10 min, a solution of 16 (1.104 g, 8.00 mmol) in DMF was directly added to the mixture followed by adding additional amount of DIEA (112 μL, 0.64 mmol). After 24 h, all resin-bound peptide was cleaved from the resin by treatment with a mixture of TFA/TIS/H2O (95%/2.5%/2.5%). After cleavage from the resin, the compound was lyophilized. The crude product was purified by reverse-phase semi-preparative HPLC and characterized by analytical HPLC and MS measurements. Yield: (0.087 g, 45%). 1H NMR of 17′ (500 MHZ, CD3OD) δ 7.80 (m, 3H), 7.68 (s, 1H), 7.44 (m, 2H), 7.39 (m, 1H), 4.65 (dd, J=8.5, 6.5 Hz, 1H), 4.31 (dd, J=8.5, 5.0 Hz, 1H), 4.28 (dd, J=8.7, 5.8 Hz, 1H), 4.18 (dd, J=8.55, 4.7 Hz, 1H), 3.88 (bs, 6H), 3.28 (m, 16H), 3.24 (m, 5H), 3.16-2.94 (m, 6H), 2.83 (s, 4H), 2.66 (m, 2H), 2.41 (m, 2H), 2.31 (m, 2H), 2.14 (m, 2H), 1.89 (m, 1H), 1.78 (m, 3H), 1.70 (m, 2H), 1.62 (m, 5H), 1.54 (m, 3H), 1.37 (m, 6H), 1.26 (m, 3H), 0.92 (m, 2H). HRESIMS [M+H]+ calcd for C₆₅H₉₅N₁₂O₂₂ 1395.6684, found 1395.6680.

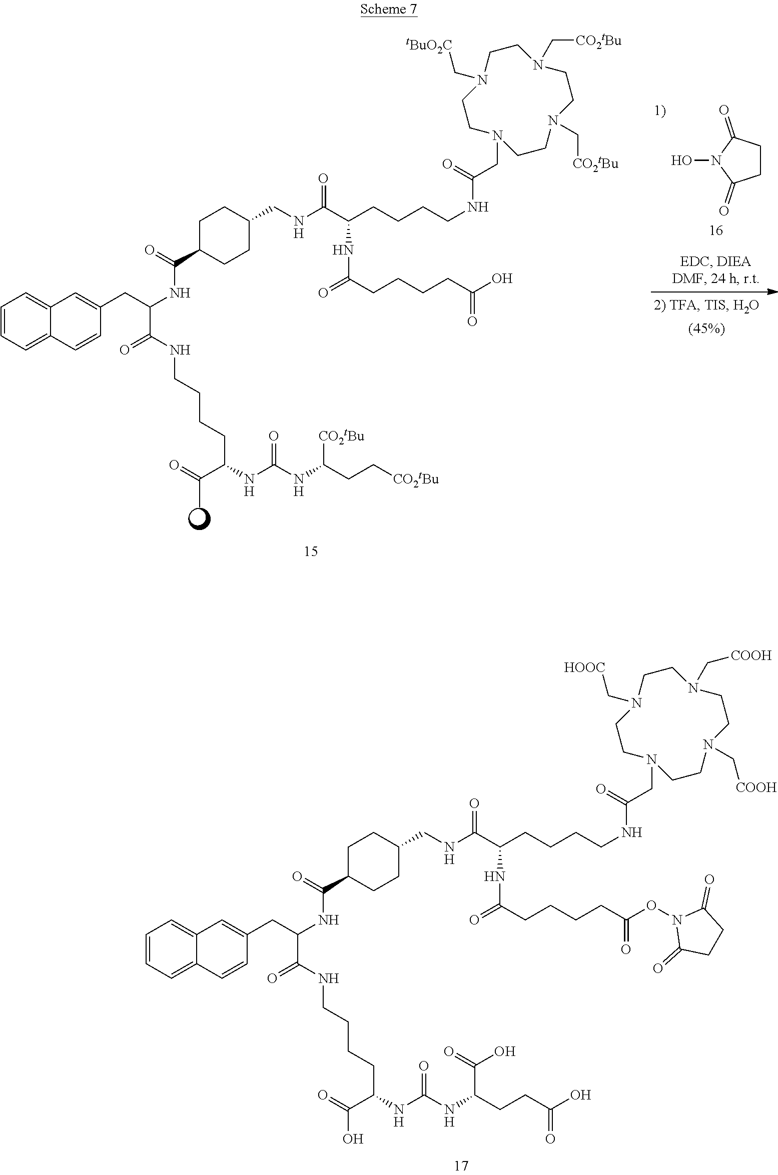 [SMILES: CC(=O)[C@H](CCCCNC(=O)C(Cc1ccc2ccccc2c1)NC(=O)[C@H]1CC[C@H](CNC(=O)[C@H](CCCCNC(=O)CN2CCN(CC(=O)OC(C)(C)C)C(C)CN(CC(=O)OC(C)(C)C)CCN(CC(=O)OC(C)(C)C)CC2C)NC(=O)CCCCC(=O)O)CC1)NC(=O)N[C@@H](CCC(=O)OC(C)(C)C)C(=O)OC(C)(C)C ; O=C1CCC(=O)N1O ; CC1CN(CC(=O)O)CCN(CC(=O)O)CC(C)N(CC(=O)NCCCC[C@H](NC(=O)CCCCC(=O)ON2C(=O)CCC2=O)C(=O)NC[C@H]2CC[C@H](C(=O)NC(Cc3ccc4ccccc4c3)C(=O)NCCCC[C@H](NC(=O)N[C@@H](CCC(=O)O)C(=O)O)C(=O)O)CC2)CCN1CC(=O)O]

The synthesis of compound 20 as shown in Scheme 8 was synthesized starting from compound 18 with the introduction of compound 19 according to the following: To 19 (38 mg, 0.087 mmol) in 0.5 mL of DMF under Argon was added DIPEA (0.053 mL, 0.304 mmol) followed by 18 (71.8 mg, 0.105 mmol) dissolved in 0.5 mL DMF, and allowed to stir at room temperature for 4 h. The reaction mixture was then concentrated under reduced pressure and purified using flash chromatography (dichloromethane:methanol 95:5 to 4:1) to give 20 as a white solid (59.4 mg, 0.065 mmol, 75.7%). 1H NMR of 20 (500 MHZ, (CD3)2SO) δ 10.00 (s, 1H), 8.19 (d, J=7.0 Hz, 1H), 7.89 (d, J=7.4 Hz, 2H), 7.75 (m, 4H), 7.57 (t, J=8.5 Hz, 3H), 7.42 (m, 3H), 7.33 (t, J=16.8, 9.9 Hz, 3H), 7.27 (d, J=8.5 Hz, 2H), 7.21 (d, J=8.2 Hz, 2H), 7.04 (t, J=8.2 Hz, 1H), 6.99 (d, J=8.2 Hz, 2H), 6.64 (bs, 2H), 5.83 (s, 2H), 4.94 (s, 2H), 4.42 (quin, J=14.0, 7.0 Hz, 1H), 4.30 (m, 1H), 4.22 (m, 2H), 4.13 (m, 2H), 3.93 (m, 1H), 2.89 (t, J=7.3 Hz, 2H), 2.00 (sextet, J=13.6, 6.7 Hz, 1H), 1.70 (quin, J=15.3, 7.5 Hz, 2H), 1.38 (sextet, J=15.1, 7.4 Hz, 2H), 1.30 (d, J=7.2 Hz, 3H), 0.87 (m, 9H). ESIMS [M+H]+ calcd for C₅₃H₅₇N₈O₆ 901.44, found 901.40.

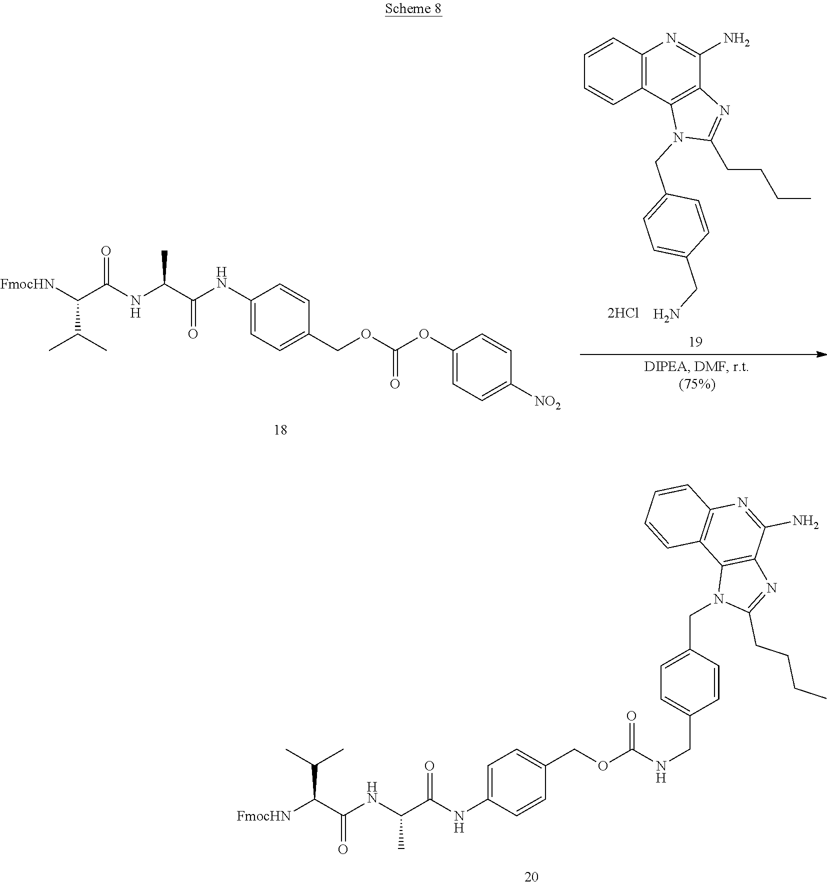

The synthesis of compound 21 as shown in Scheme 9 was synthesized starting from compound 20 according to the following: To 20 (69.4 mg, 0.077 mmol) in 0.5 mL of DMF was added piperidine (0.038 mL, 0.38 mmol) and allowed to stir at room temperature for 30 min. The reaction mixture was then loaded on to a 10 g reversed phase sep-pak (which had been washed with 100 mL of acetonitrile followed by 100 mL of water). Once loaded the column was eluted with water, 8:2, 7:3, 6:4, 5:5, 4:6 and 3:7 (H2O:MeCN, acidified to 0.001M using HCl) in 20 mL fractions. The fraction eluted with 6:4 H2O:MeCN contained 21 as confirmed by LCMS. This fraction was concentrated under reduced pressure and lyophilized overnight to yield 21 as the HCl salt (20.2 mg, 0.028 mmol, 36.6%). HRESIMS [M+H]+ calcd for C₃₈H₁₄₇N₈O₄ 679.3720, found 679.3716.

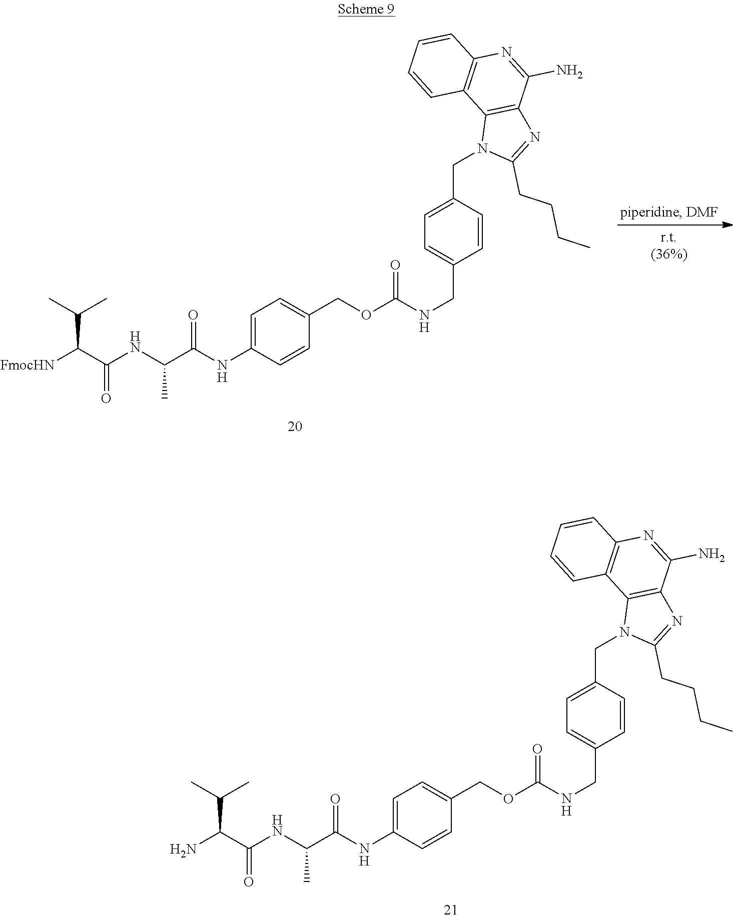

The synthesis of exemplary un-labeled RIMS-1 as shown in Scheme 10 was synthesized starting from compound 21 in the presence of compound 17 according to the following: To 17 (20 mg, 0.014 mmol) in DMF was added 21 (20.0 mg, 0.027 mmol) in DMF dropwise. After 5 min, DIEA (112 μL, 0.14 mmol) was added to the mixture and the reaction was allowed to stir for 48 h. After all starting materials were converted, the solvent was evaporated. The crude product was purified by reversed phase semi-preparative HPLC and characterized by analytical HPLC and MS measurements. Yield: (0.017 g, 61%). 1H NMR (700 MHZ, (CD3)2SO) d 8.41 (bs, 1H), 8.20 (d, J=6.9 Hz, 1H), 7.95 (m, 3H), 7.85 (d, J=7.8 Hz, 2H), 7.79 (m, 5H), 7.75 (t, J=5.9 Hz, 1H), 7.67 (s, 1H), 7.62 (t, J=7.7 Hz, 1H), 7.57 (, d, J=8.6 Hz, 2H), 7.45 (m, 2H), 7.38 (m, 2H), 7.26 (d, J=8.5 Hz, 2H), 7.20 (d, J=8.5 Hz, 2H), 7.03 (d, J=8.2 Hz, 2H), 6.54 (bs, 10H), 6.32 (d, J=8.1 Hz, 1H), 6.28 (d, J=8.1 Hz, 1H), 5.93 (s, 1H), 4.92 (s, 1H), 4.54 (m, 1H), 4.38 (p, 4.38, J=7 Hz, 1H), 4.19 (m, 2H), 4.14 (d, J=6.1 Hz, 2H), 4.09 (m, 2H), 4.00 (m, 2H), 3.84 (m, 4H), 3.08 (m, 16H), 2.96 (t, J=7.7 Hz, 3H), 2.92 (m, 2H), 2.85 (m, 2H), 2.23 (m, 3H), 2.11 (m, 3H), 2.06 (m, 1H), 1.96 (m, 2H), 1.72 (m, 3H), 1.64 (m, 2H), 1.58 (m, 4H), 1.44 (m, 8H), 1.39 (m, 4H), 1.29 (m, 6H), 1.22 (m, 8H), 1.14 (m, 1H), 1.02 (m, 1H), 0.86 (m, 6H), 0.82 (d, J=6.7 Hz, 3H), 0.748 (m, 2H). MALDI-TOF [M+H]+ calcd for C₉₉H₁₃₆N₁₉O₂₃ 1960.0090, found 1960.374.

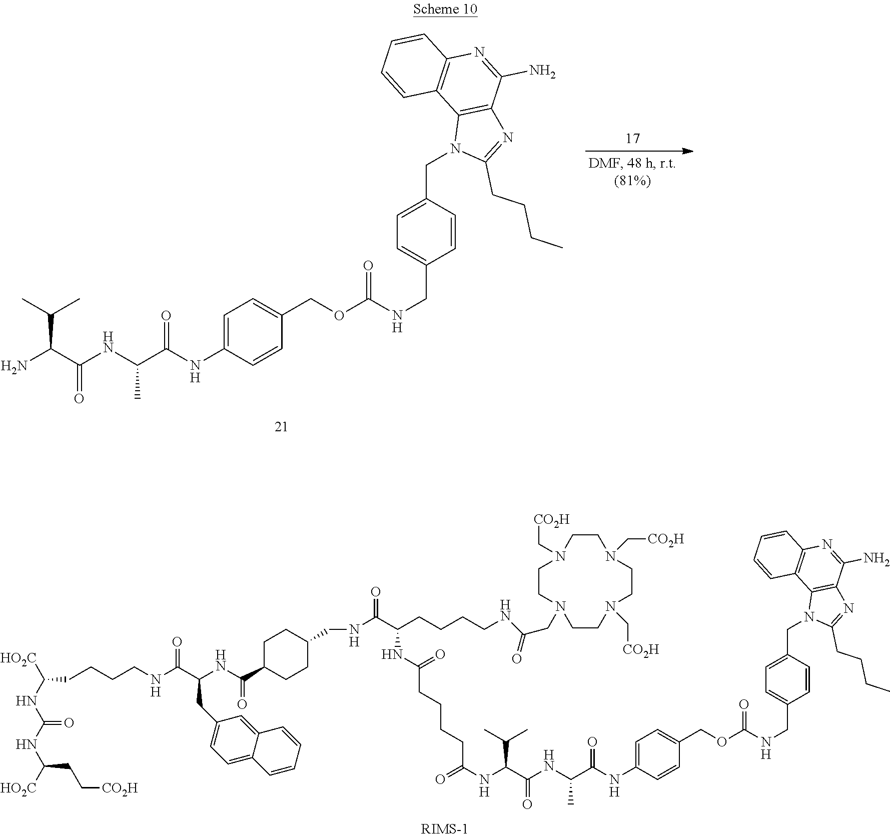

The synthesis of exemplary ^{nat}Lu-RIMS-1 as shown in Scheme 11 was synthesized starting from compound 21 in the presence of compound 17 according to the following: RIMS-1 (10 mg, 0.007 mmol) was dissolved in 100 μL of DMSO and 0.99 mL of 0.4 M NH₄OAc buffer was added. Next, LuCl₃·6H2O (5.45 mg, 0.014 mmol) was added to the reaction mixture. After 5 min, the reaction mixture was heated up to 80° C. for 1 hour. The solvent was evaporated, and the crude product was purified by reverse-phase semi-preparative HPLC and characterized by analytical HPLC and MS measurements. Yield: (0.01 g, 65%). MALDI-TOF [M+H]+ calculated for C₉₉H₁₃₃LuN₁₉O₂₃ 2130.9229, found 2130.079.

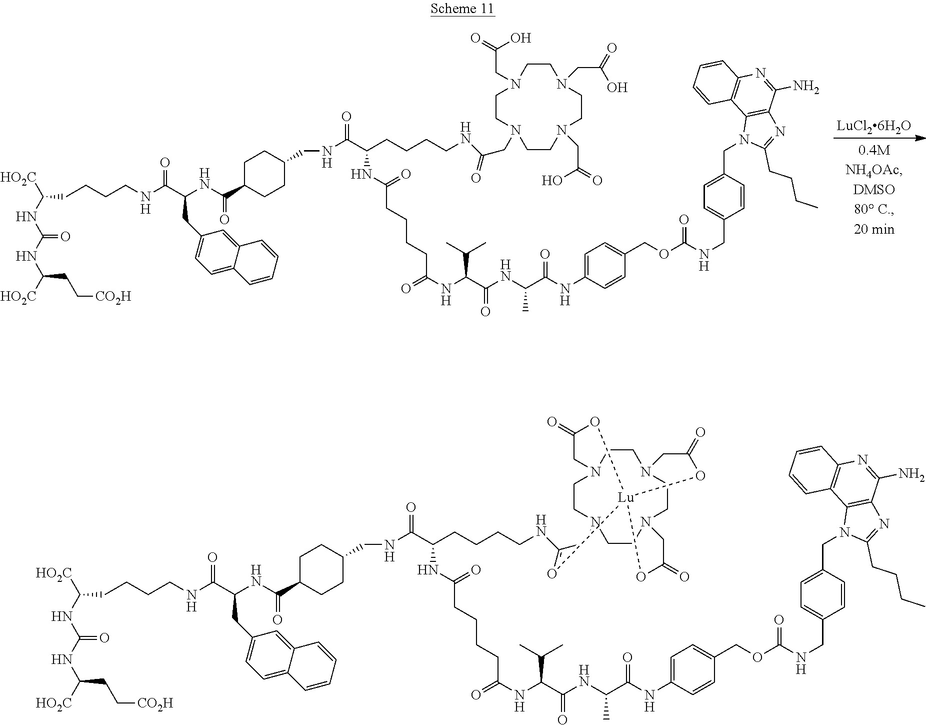

### Example 2: Synthesis and Characterization of Control Compounds

Control compounds PSMA-617 and ⁸⁹Zr-DFO-anti-CD3 were prepared and characterized according to conventional methods including MALDI-TOF MS spectrometry.

### Example 3: Stability Analysis of ¹⁷⁷Lu-RIMS-1

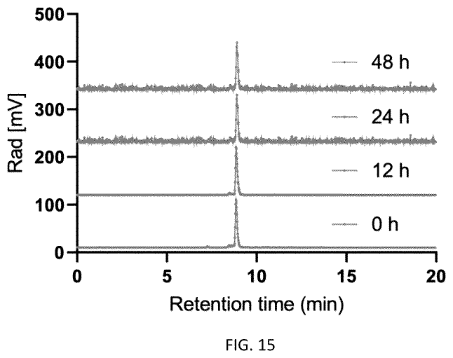

The stability for ¹⁷⁷Lu-RIMS-1 was evaluated by radio-HPLC as shown in FIG. 15 for radiolytic degradation and decomplexation at relevant time point concentrations for dose preparation, storage, and administration. ¹⁷⁷Lu-RIMS-1 was radiolabeled as previously described with a specific activity of 3.7 MBq/nmol and diluted in PBS. A 0.1 mL aliquot was taken, stored at room temperature and tested for stability by radio-HPLC at 12 h, 24 h and 48 h.

### Example 4: Animal Studies

### Example 4.1 hPSMA Expression in RM1-PGLS Tumors In Vivo with Pet Imaging

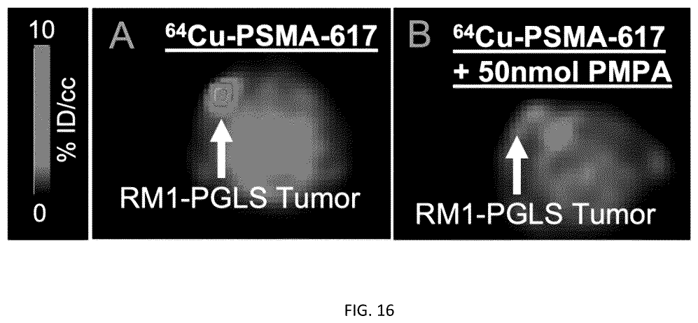

C57BL/6J mice bearing RM1-PGLS tumors (50-135 mm³) where randomized based on tumor volumes into 2 groups and administered compound intravenously. ⁶⁴Cu-PSMA-617 was synthesized according to conventional synthetic methods. (1) Mice in group 1 received 5.0 MBq of 64Cu-PSMA-617 (2 nmol, ⁶⁴Cu supplied by the University of Wisconsin in 0.1 M HCl) by intravenously tail-vein catheter. Mice in group 2 received 50 nmol of the PSMA inhibitor, 2-PMPA formulated in sterile PBS pH 7.4 followed by 5.0 MBq of ⁶⁴Cu-PSMA-617 (2 nmol). Mice were imaged at 90 min post-injection using Siemens Inveon PET/CT and images were reconstructed using ASIPro software. Region of interest (ROI) analyses on all images were performed using AMIDE with PET imaging results and analyses are shown in FIG. 16.

### Example 4.2 Post-RIMS Treatment Analyses of Murine Subjects Bearing RM1-PGLS Tumors

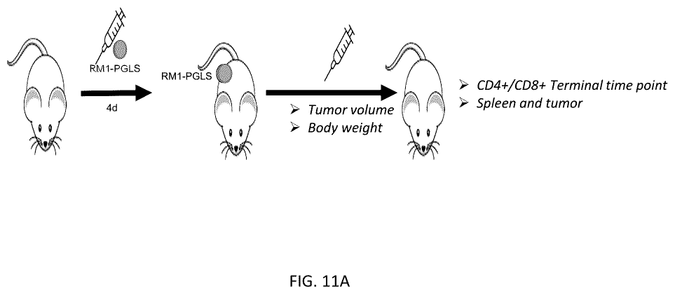

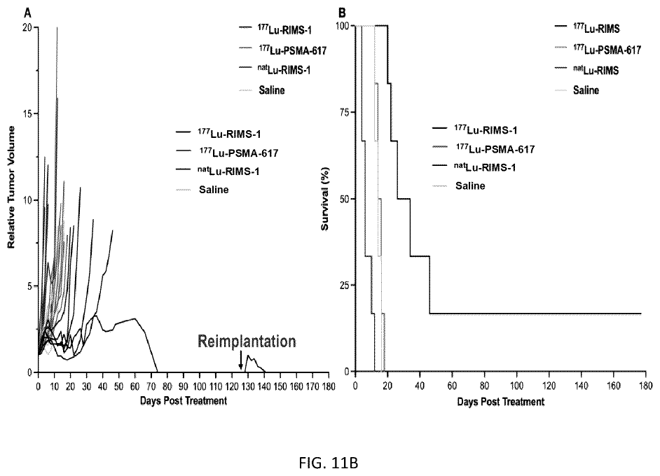

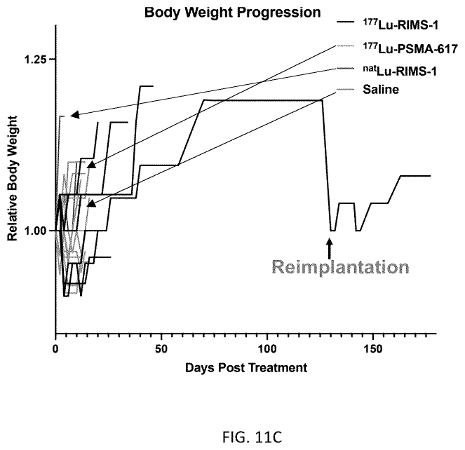

Murine subjects were treated with RIMS compounds and tumor volume (FIG. 11B, left), subject viability measured as percent survival (FIG. 11B, right), subject weight (FIG. 11C), were described and analyzed as shown in FIGS. 11A-11C. Overall survival for control RIMS compounds in comparison to exemplary novel ¹⁷⁷Lu-RIMS-1 are shown in Table 1. Median overall survivability (OS) in murine subjects treated with RIMS compounds and control therapies.

TABLE 1
Overall Survival of Murine Subjects in vivo post treatment

| Compound | Median OS |
|---|---|
| ¹⁷⁷Lu-RIMS-1 | 30 d |
| | (P < 0.0007) |
| ¹⁷⁷Lu-PSMA-617 | 15 d |
| ^{nat}Lu-RIMS-1 | 6 d |
| Vehicle | 14 d |

### Example 5. Biodistribution of ¹⁷⁷Lu-RIMS-1 vs ¹⁷⁷Lu-PSMA-617

Biodistribution analyses of ¹⁷⁷Lu-RIMS-1 and ¹⁷⁷Lu-PSMA-617 were carried out in a xenograft model of prostate cancer with a PC-3 PiP tumor (PSMA(+)) on the left shoulder of a murine subject, and a PC-3 flu tumor (PSMA(−)) on the right should of a murine subject. Murine subjects examined were Male NCr nude mice. Biodistribution results are shown in Table 2-Table 6.

TABLE 2
Biodistribution of 177Lu-RIMS-1 and 177Lu-PSMA-617 in a syngeneic
model of prostate cancer with a RM1-PGLS tumor on the right shoulder in C57BL/6J mice

| | ¹⁷⁷Lu-RIMS-1 | | | ¹⁷⁷Lu-PSMA-617 | | |
|---|---|---|---|---|---|---|
| | (% ID/g ± SD) | | | (% ID/g ± SD) | | |
| Organ | 2 h | 24 h | 72 h | 2 h | 24 h | 72 h |
| Blood | 2.28 ± 0.29 | 0.11 ± 0.01 | 0.2 ± 0.00 | 0.03 ± 0.02 | 0.00 ± 0.00 | 0.01 ± 0.01 |
| Heart | 0.83 ± 0.09 | 0.13 ± 0.11 | 0.03 ± 0.01 | 0.03 ± 0.01 | 0.01 ± 0.00 | 0.01 ± 0.00 |
| Liver | 23.35 ± 6.93 | 8.69 ± 1.53 | 7.28 ± 1.06 | 0.17 ± 0.12 | 0.02 ± 0.01 | 0.02 ± 0.01 |
| Spleen | 10.31 ± 2.96 | 2.16 ± 0.73 | 1.14 ± 0.36 | 0.08 ± 0.03 | 0.02 ± 0.01 | 0.02 ± 0.01 |
| Kidney | 15.27 ± 3.61 | 5.03 ± 2.20 | 1.66 ± 0.37 | 1.39 ± 0.48 | 0.09 ± 0.07 | 0.04 ± 0.01 |
| Sm Int | 0.55 ± 0.30 | 0.07 ± 0.02 | 0.05 ± 0.01 | 0.03 ± 0.01 | 0.01 ± 0.01 | 0.02 ± 0.01 |
| Muscle | 0.27 ± 0.08 | 0.04 ± 0.03 | 0.02 ± 0.01 | 0.05 ± 0.05 | 0.01 ± 0.01 | 0.01 ± 0.00 |
| Bone | 1.03 ± 0.43 | 0.24 ± 0.10 | 0.14 ± 0.02 | 0.05 ± 0.05 | 0.01 ± 0.01 | 0.01 ± 0.01 |
| RM1- | 1.98 ± 0.37 | 0.66 ± 0.24 | 0.23 ± 0.06 | 0.80 ± 0.18 | 0.25 ± 0.09 | 0.18 ± 0.06 |
| PGLS | | | | | | |

TABLE 3
Biodistribution of 177Lu-RIMS-1 and 177Lu-PSMA-617 in a xenograft model
of prostate cancer with a PC-3 PiP tumor (PSMA(+)) on the left shoulder and a PC-3 flu tumor
(PSMA(−)) on the right shoulder in Male NCr nude mice

| | ¹⁷⁷Lu-RIMS-1 | | | ¹⁷⁷Lu-PSMA-617 | | |
|---|---|---|---|---|---|---|
| | (% ID/g ± SD) | | | (% ID/g ± SD) | | |
| Organ | 2 h | 24 h | 72 h | 2 h | 24 h | 72 h |
| Blood | 3.36 ± 0.2 | 0.06 ± 0.01 | 0.01 ± 0.01 | 0.95 ± 0.53 | 0.21 ± 0.13 | 0.03 ± 0.01 |
| Heart | 1.28 ± 0.08 | 0.16 ± 0.02 | 0.06 ± 0.01 | 0.27 ± 0.12 | 0.09 ± 0.05 | 0.03 ± 0.01 |
| Liver | 7.95 ± 0.35 | 3.19 ± 0.38 | 1.36 ± 0.29 | 0.29 ± 0.14 | 0.08 ± 0.040 | 0.03 ± 0.01 |
| Spleen | 2.32 ± 0.052 | 0.77 ± 0.31 | 0.29 ± 0.11 | .027 ± 0.14 | 0.08 ± 0.040 | 0.04 ± 0.01 |
| Kidney | 24.54 ± 2.58 | 6.24 ± 1.03 | 2.10 ± 0.31 | 5.12 ± 1.95 | 0.57 ± 0.29 | 0.08 ± 0.05 |
| Sm Int | 1.15 ± 0.1 | 0.54 ± 0.8 | 0.05 ± 0.02 | 0.28 ± 0.15 | 0.06 ± 0.020 | 0.03 ± 0.01 |
| Muscle | 0.36 ± 0.06 | 0.04 ± 0.01 | 0.02 ± 0.0 | 0.14 ± 0.05 | 0.04 ± 0.010 | 0.02 ± 0.01 |
| Bone | 0.72 ± 0.15 | 0.17 ± 0.06 | 0.10 ± 0.04 | 0.21 ± 0.11 | 0.07 ± 0.010 | 0.05 ± 0.01 |
| PSMA | 7.16 ± 2.68 | 6.04 ± 1.76 | 2.65 ± 1.03 | 11.78 ± 1.46 | 8.24 ± 3.22 | 3.65 ± 0.59 |
| (+) | | | | | | |
| (PC3- | | | | | | |
| PIP) | | | | | | |
| PSMA | 1.65 ± 0.24 | 0.16 ± 0.01 | 0.07 ± 0.05 | 0.48 ± 0.2 | 0.18 ± 0.13 | 0.02 ± 0.01 |
| (−) | | | | | | |
| (PC3- | | | | | | |
| Flu) | | | | | | |

TABLE 4
Total CD8+ and CD4+ T-cell populations in the
spleen and tumor of RM1-PGLS bearing C57BL/6J mice administered
with 177Lu-RIMS-1, 177Lu-PSMA-617, natLu-RIMS-1 or vehicle

| | | ¹⁷⁷Lu-PSMA- | | |
|---|---|---|---|---|
| | ¹⁷⁷Lu-RIMS-1 | 617 | ^{nat}Lu-RIMS-1 | Vehicle |
| | Spleen | | | |
| CD4⁺ | 14.51 ± 2.24 | 12.73 ± 2.17 | 10.91 ± 3.73 | 12.33 ± 3.01 |
| CD8⁺ | 7.70 ± 0.84 | 8.78 ± 1.61 | 7.79 ± 3.25 | 9.26 ± 3.72 |
| | Tumor | | | |
| CD4⁺ | 2.07 ± 1.54 | 1.47 ± 0.59 | 1.46 ± 1.54 | 0.94 ± 0.92 |
| CD8⁺ | 4.72 ± 2.70 | 4.76 ± 1.40 | 3.83 ± 2.42 | 3.84 ± 2.17 |

TABLE 5
ROI data of 89Zr-DFO-anti-CD3 8-12 days after administration of therapy
% Injected Dose/cc of ⁸⁹Zr-DFO-anti-CD3

| Days | ¹⁷⁷Lu-RIMS-1 | ¹⁷⁷Lu-PSMA-617 | Vehicle | ^{nat}Lu-RIMS-1 |
|---|---|---|---|---|
| | Tumor | | | |
| 8 | 15.85 ± 3.12 | 10.27 ± 0.024 | 12.57 ± 4.23 | 11.89 ± 2.04 |
| 9 | 14.94 ± 2.65 | 7.83 ± 1.66 | 12.97 ± 2.08 | 12.79 ± 0.94 |
| 10 | 13.74 ± 3.74 | 6.67 ± 3.63 | 10.33 ± 1.93 | 12.92 ± 1.52 |
| 11 | 12.20 ± 4.43 | 3.90 ± 0.35 | 7.82 ± 1.97 | 12.71 ± 1.35 |
| 12 | 11.83 ± 5.22 | 2.72 ± 0.81 | 7.33 ± 3.11 | 121.70 ± 2.00 |

Spleen

| | | | | |
|---|---|---|---|---|
| 8 | 15.65 ± 0.96 | 21.55 ± 4.49 | 14.84 ± 2.96 | 19.86 ± 3.39 |
| 9 | 19.39 ± 3.46 | 24.84 ± 4.91 | 19.93 ± 4.85 | 21.09 ± 6.75 |
| 10 | 18.45 ± 1.30 | 27.09 ± 3.75 | 20.61 ± 6.40 | 19.71 ± 3.40 |
| 11 | 19.06 ± 2.34 | 23.82 ± 3.55 | 21.44 ± 7.78 | 19.34 ± 3.28 |
| 12 | 18.54 ± 1.04 | 24.65 ± 0.81 | 21.73 ± 6.87 | 21.10 ± 3.90 |

Blood

| | | | | |
|---|---|---|---|---|
| 8 | 7.95 ± 0.75 | 8.94 ± 0.34 | 6.75 ± 0.17 | 7.32 ± 0.87 |
| 9 | 7.075 ± 0.94 | 5.85 ± 2.22 | 6.53 ± 1.25 | 5.89 ± 1.72 |
| 10 | 4.98 ± 0.61 | 4.13 ± 0.68 | 5.42 ± 1.29 | 3.71 ± 0.53 |
| 11 | 4.33 ± 1.40 | 4.05 ± 1.24 | 4.04 ± 1.34 | 3.73 ± 0.65 |
| 12 | 3.07 ± 1.23 | 4.26 ± 0.62 | 4.15 ± 1.21 | 3.55 ± 0.40 |

TABLE 6
OLINDA Results: Dosimetry to Adult Male
from Mouse biodistribution (μSv/MBq)

| Target Organ | ¹⁷⁷Lu-RIMS-1 | ¹⁷⁷Lu-PSMA-617 |
|---|---|---|
| Adrenals | 8.71 | 1.32 |
| Brain | 0.02 | 0.00 |
| Esophagus | 2.03 | 0.25 |
| Eyes | 0.02 | 0.00 |
| Gallbladder Wall | 7.09 | 0.93 |
| Left Colon | 1.26 | 0.18 |
| Small Intestine | 3.60 | 1.54 |
| Stomach Wall | 2.02 | 0.23 |
| Right Colon | 1.94 | 0.27 |
| Rectum | 0.25 | 0.07 |
| Heart Wall | 16.60 | 1.57 |
| Kidneys | 262.00 | 54.70 |
| Liver | 219.00 | 27.80 |
| Lungs | 15.10 | 2.43 |
| Pancreas | 2.49 | 0.32 |
| Prostate | 0.34 | 0.10 |
| Salivary Glands | 0.05 | 0.01 |
| Red Marrow | 1.39 | 0.10 |
| Osteogenic Cells | 3.70 | 0.83 |
| Spleen | 228.00 | 0.25 |
| Tumor | 237.00 | 145.00 |
| Thymus | 0.95 | 0.11 |
| Thyroid | 0.36 | 0.05 |
| Urinary Bladder Wall | 10.90 | 7.66 |
| Today Body | 7.73 | 1.13 |

### Example 4.2 Immune Cell Recruitment

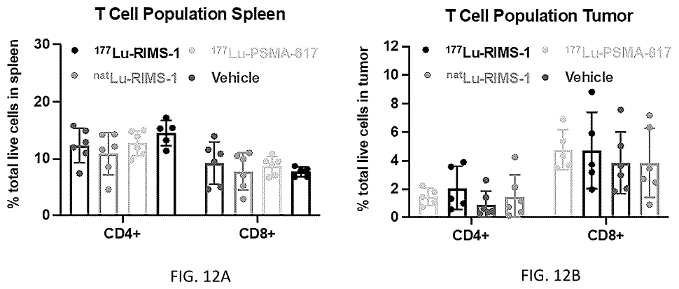

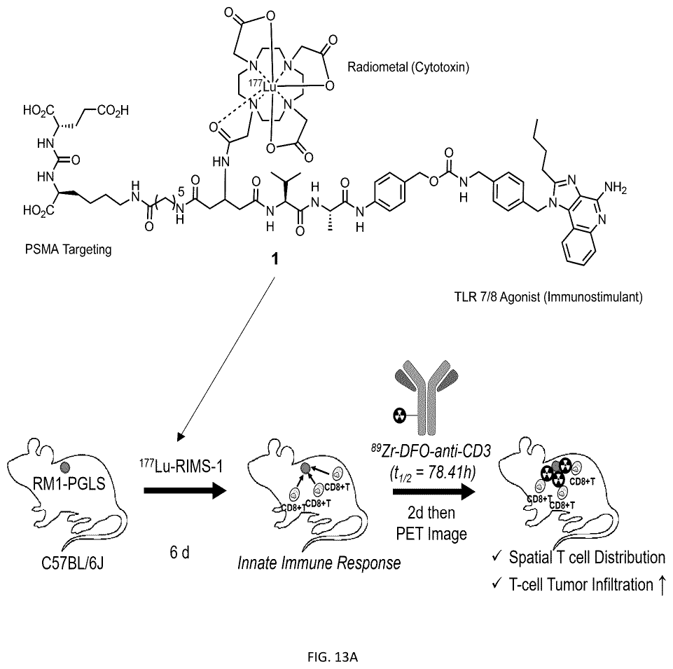

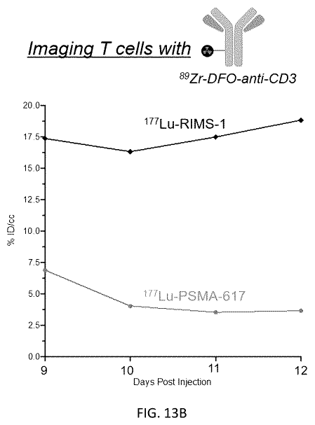

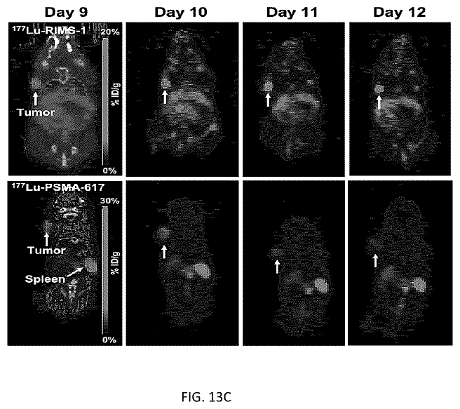

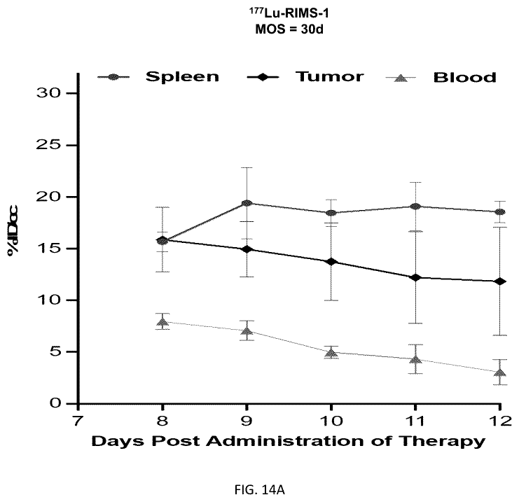

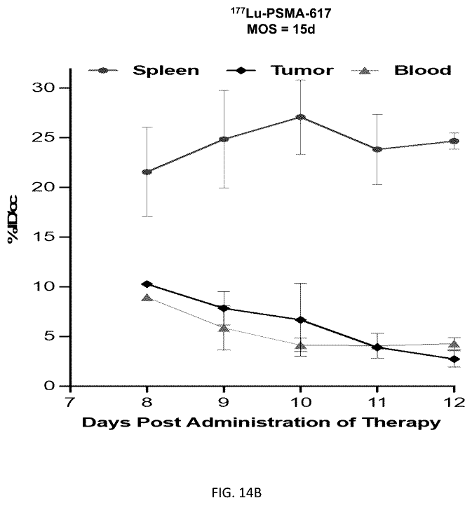

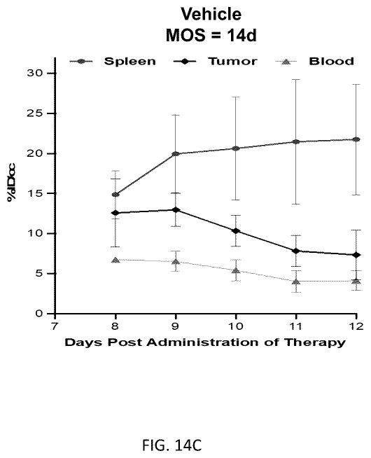

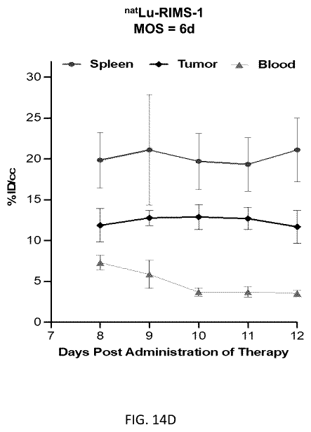

¹⁷⁷Lu-RIMS-1 and control compounds were evaluated for immune cell recruitment. T-cell populations were measured in the spleen and in the tumor as shown in FIG. 12A-12B.

### Example 6. Physiochemical Analysis of RIMS-1

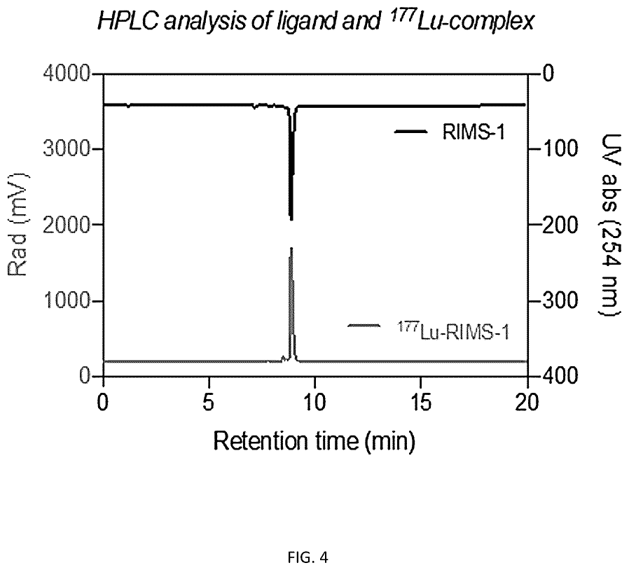

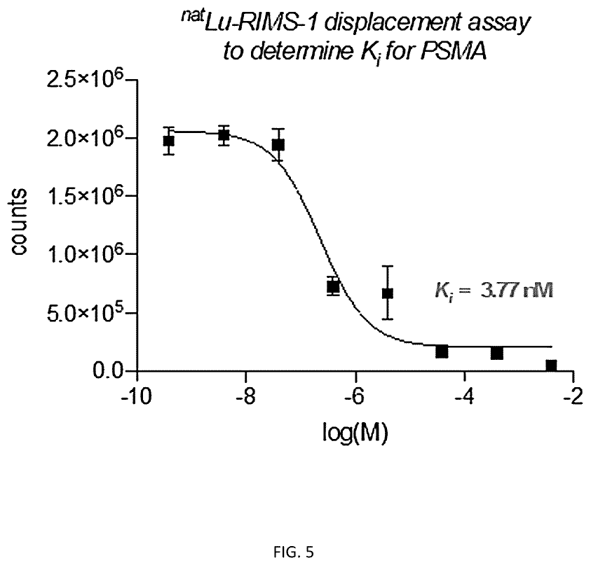

RIMS-1 was subjected to analysis prior to use in animal studies. HPLC analysis (FIG. 4) of the uncomplexed RIMS-1 with ¹⁷⁷Lu-RIMS-1 showed the same retention time, showing the labeled ¹⁷⁷Lu-complex is consistent with the un-complexed precursor. A displacement assay was conducted using PSMA expressing cells in the presence of ¹⁷⁷Lu-RIMS-1 (see FIG. 5). Half-life of the cleaved immunostimulant moiety from ¹⁷⁷Lu-RIMS-1 was also analyzed using conventional methods and found to have a t_{1/2}=8.8 hours.

### Example 7. Dosing Analysis for Total Administered Radioactivity

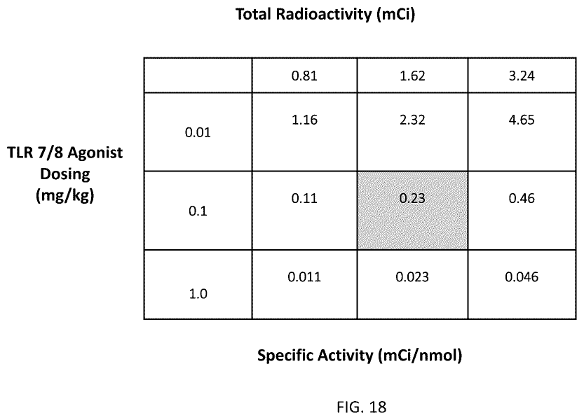

Non-therapeutic doses of ¹⁷⁷Lu-RIMS-1 were administered to analyze the effect of increased total administered radioactivity on treatment outcomes as shown in in FIG. 18.

### Example 8. Additional Exemplary Synthetic Schemes

Embodiments of the compositions described herein are synthesized according to Schemes 1-7 as well as methods known to those of skill in the art.

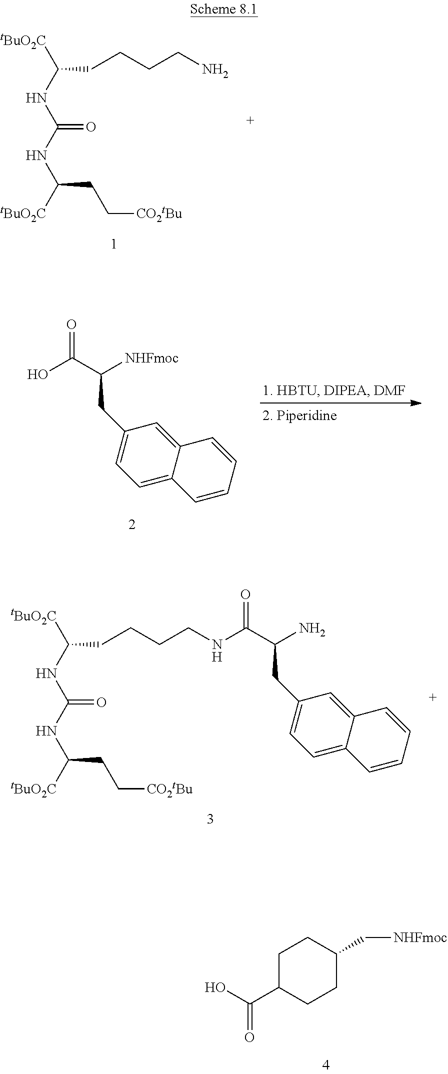

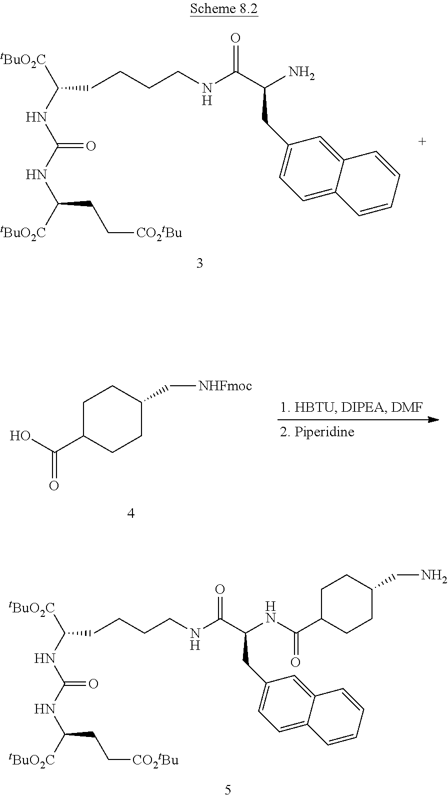

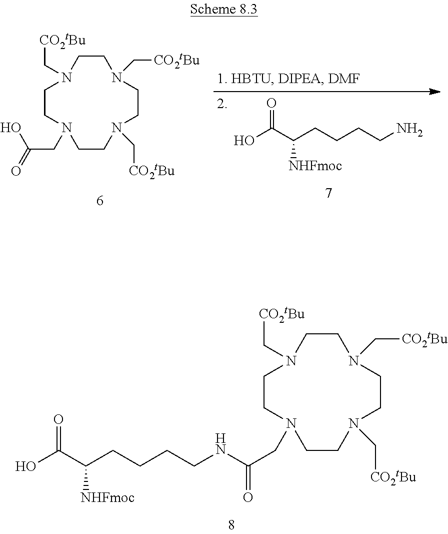

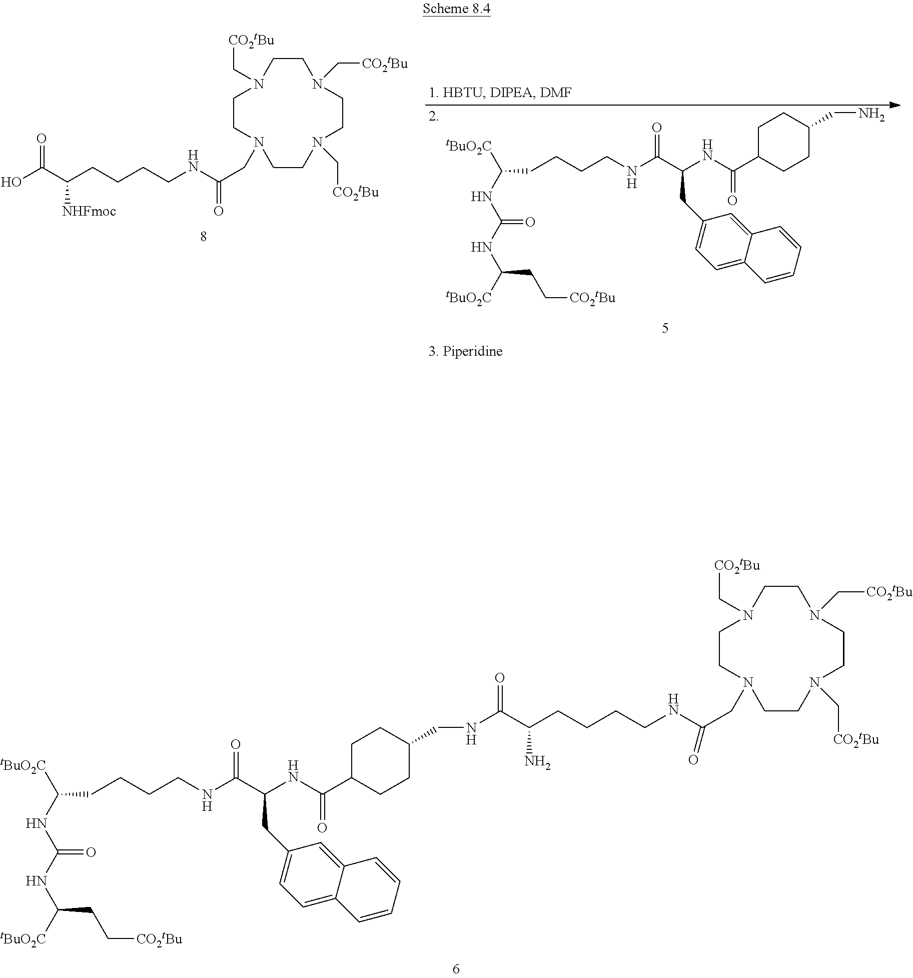

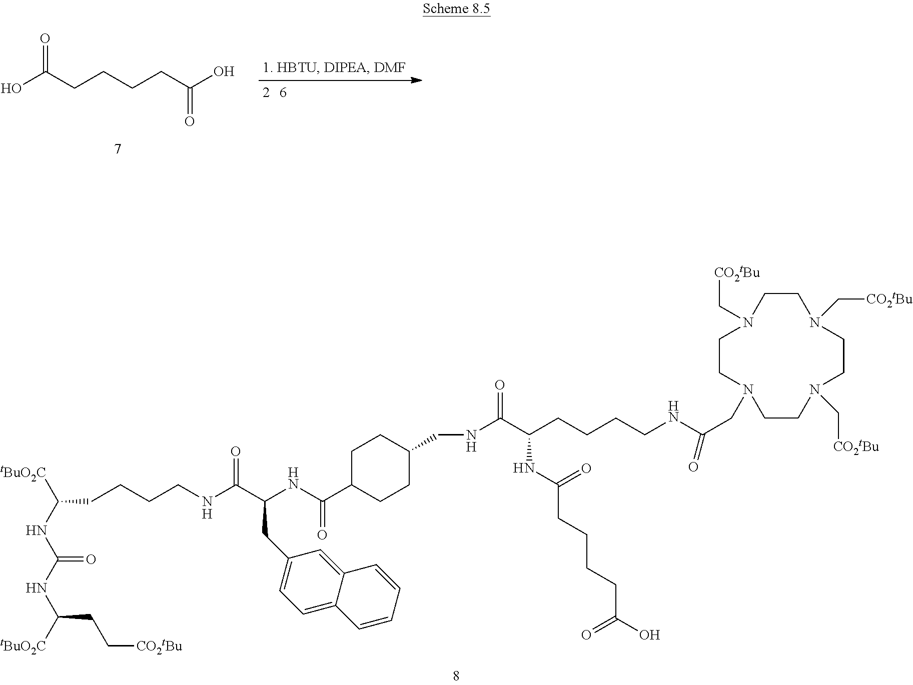

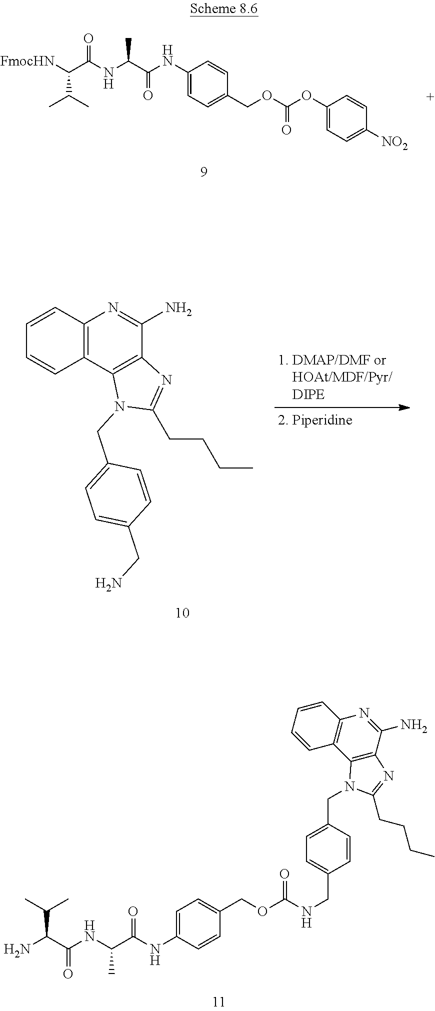

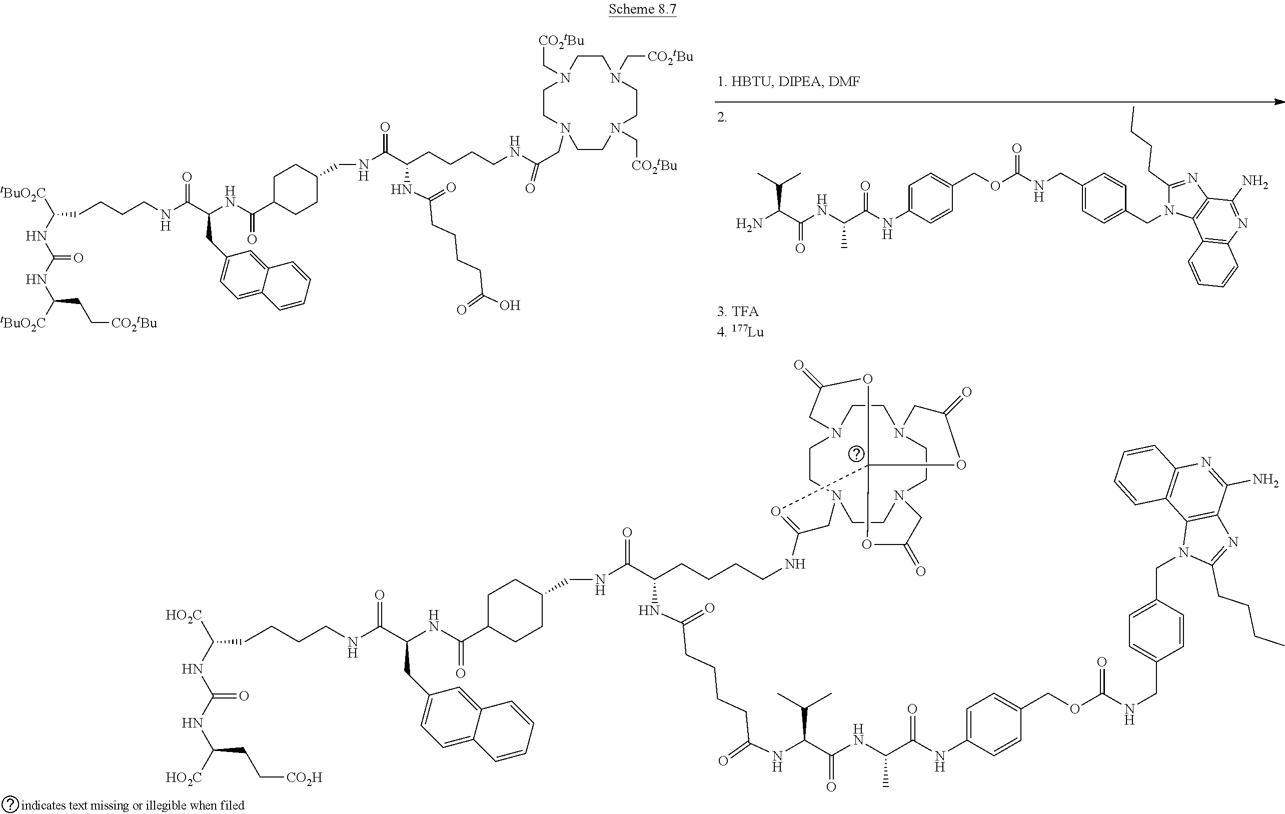

While the disclosure has been shown and described with reference to certain embodiments of the present disclosure thereof, it will be understood by those skilled in the art that various changes in form and details may be made therein without departing from the spirit and scope of the present disclosure and equivalents thereof. As one of skill in the art will readily appreciate, this disclosure has been presented for purposes of illustration and description. The disclosure above is not intended to limit the invention to the form or forms disclosed herein. Although the description of the disclosure has included description of one or more embodiments and certain variations and modifications, other variations and modifications are within the scope of the present disclosure.

1. A compound of Formula I: 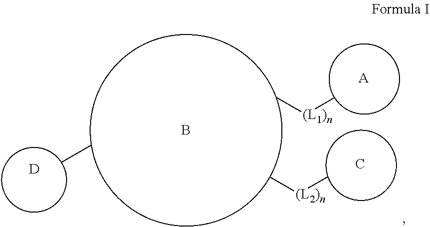
a diastereomer or enantiomer thereof, or a pharmaceutically acceptable salt of any of the foregoing, or a deuterated derivative or any of the forgoing, or a radioisotope of any of the forgoing, wherein:
A, L₁, B, D, L₂, and C groups are each independently covalently connected as one molecule;
A comprises a: taxoid, vinca alkaloid, anthracycline, maytansinoid, tubulysin, auristatin, exatecan, duocarmycin, Seco-Cyclopropabenzindol-4-One dimer or monomer, pyrrolobenzodiazepine dimer or monomer, hemiasterlin, ²¹²Pb-DOTA, ¹⁷⁷Lu-DOTA (1,4,7,10-Tetraazacyclododecane-1,4,7,10-tetraacetic acid), ⁹⁰Y-DOTA, ²²⁵Ac-DOTA, ⁴⁷Sc-DOTA, ⁶⁷Cu-DOTA, ¹³¹I-L-Tyrosine, or any combination thereof;
when A is ¹⁷⁷Lu-DOTA (1,4,7,10-Tetraazacyclododecane-1,4,7,10-tetraacetic acid), ⁹⁰Y-DOTA, ²¹²Pb-DOTA, ²²⁵Ac-DOTA, ⁴⁷Sc-DOTA, ⁶⁷Cu-DOTA, ¹³¹I-L-Tyrosine, or any combination thereof, A is covalently bound to L₁ by a C—C or C—N bond;
L₁ and L₂ are each independently selected from: (CH₂)ₙ, a dipeptide, a tripeptide, a disulfide, a hydrazone, a beta glucan, 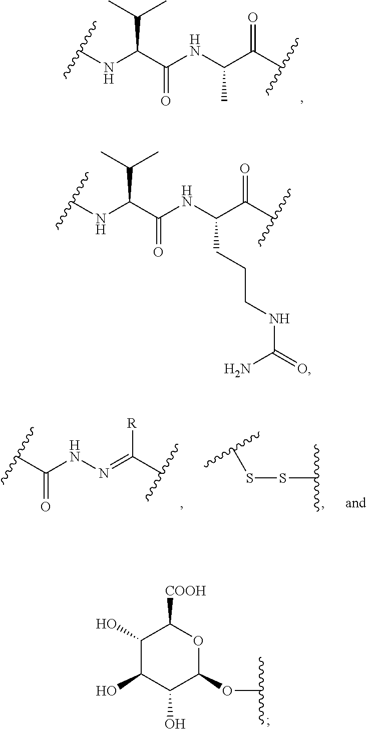
R is an aliphatic or unsaturated C1-C10, or an aromatic C6 or C10 group, wherein each R is optionally substituted with an amide, an ester, a C3-C10 cycloalkyl, an ether, or C1-C4 group;
each n is independently an integer 0-8;
B is selected from the group consisting of: 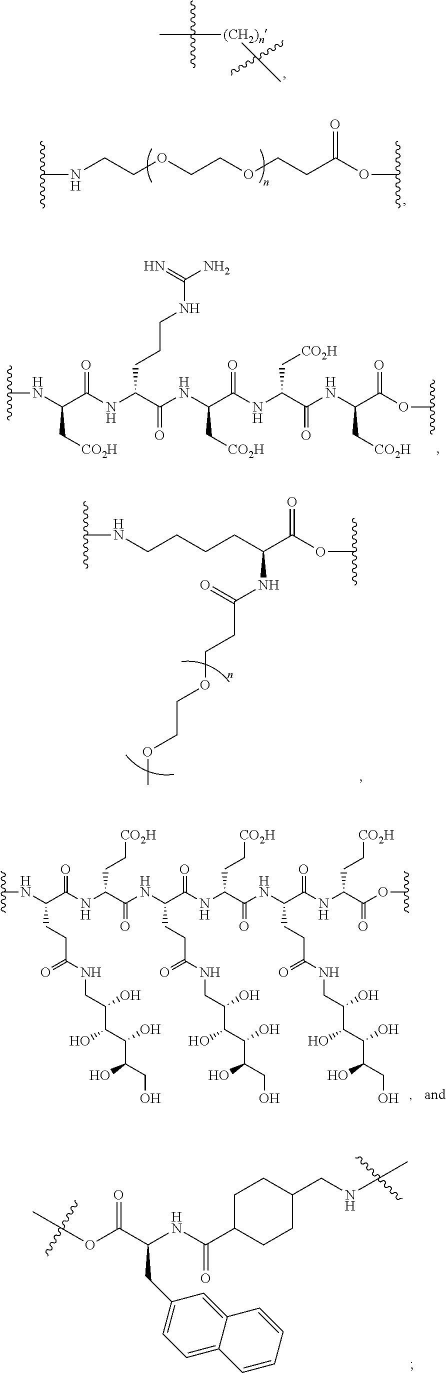
C is an immunostimulant toll-like receptor (TLR) ligand comprising a: TLR2 agonist, TLR3 agonist, TLR4 agonist, TLR5 agonist, TLR6 agonist, TLR7 agonist, TLR7/8 agonist, TLR8 agonist, TLR9 agonist, TLR10 agonist, nucleotide oligomerization domaine (NOD)-like receptor ligand, retinoic acid-inducible (RIG)-like receptor ligand, C-type lectin receptor (CLR) ligand, a cytosolic dsDNA sensor (CDS) ligand, inflammasome inducer or stimulator of interferon genes (STING) agonist, 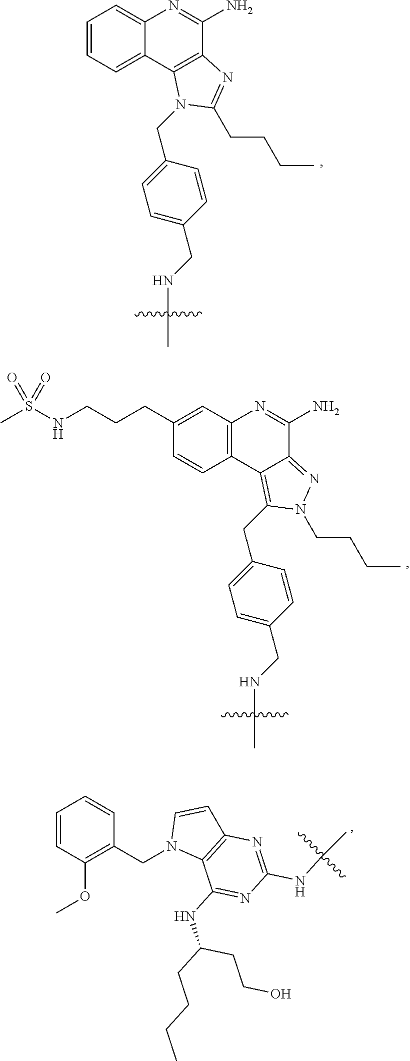 [SMILES: *NCc1ccc(Cn2c(CCCC)nc3c(N)nc4ccccc4c32)cc1 ; *NCc1ccc(Cc2c3c(nn2CCCC)c(N)nc2cc(CCCNS(C)(=O)=O)ccc23)cc1 ; *Nc1nc(N[C@H](CCO)CCCC)c2c(ccn2Cc2ccccc2OC)n1] 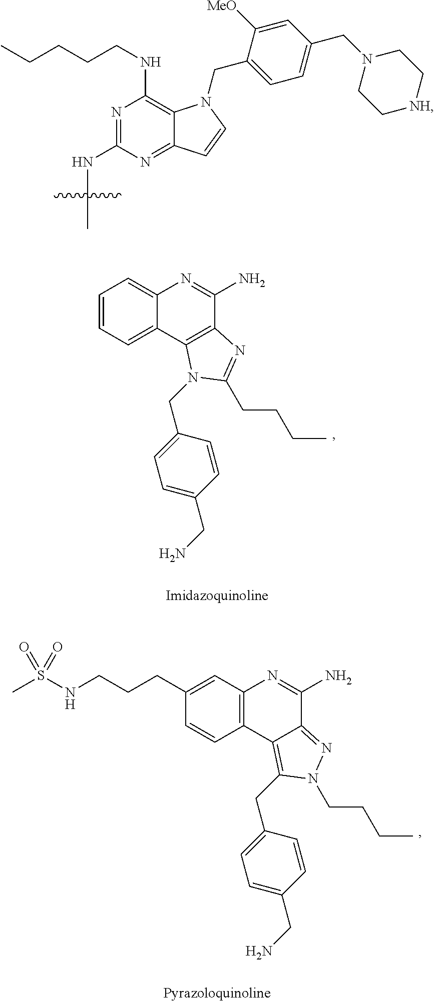 [SMILES: *Nc1nc(NCCCCC)c2c(ccn2Cc2ccc(CN3CCNCC3)cc2OC)n1 ; CCCCc1nc2c(N)nc3ccccc3c2n1Cc1ccc(CN)cc1 ; CCCCn1nc2c(N)nc3cc(CCCNS(C)(=O)=O)ccc3c2c1Cc1ccc(CN)cc1] 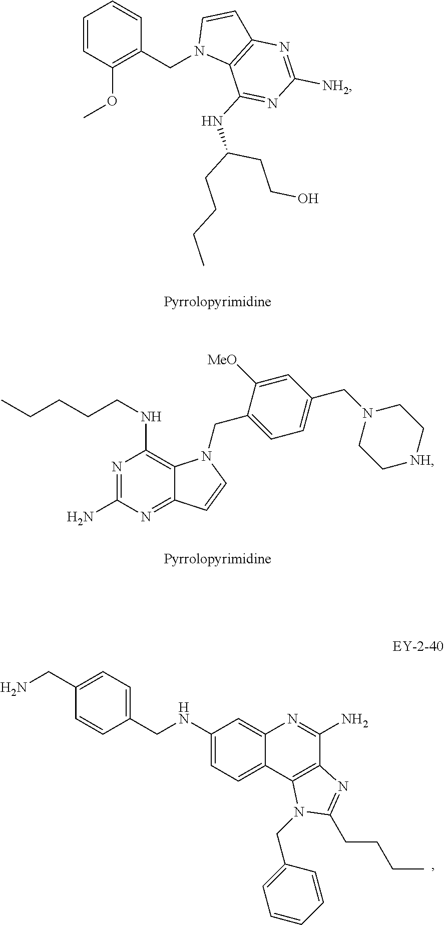 [SMILES: CCCC[C@@H](CCO)Nc1nc(N)nc2ccn(Cc3ccccc3OC)c12 ; CCCCCNc1nc(N)nc2ccn(Cc3ccc(CN4CCNCC4)cc3OC)c12 ; CCCCc1nc2c(N)nc3cc(NCc4ccc(CN)cc4)ccc3c2n1Cc1ccccc1] 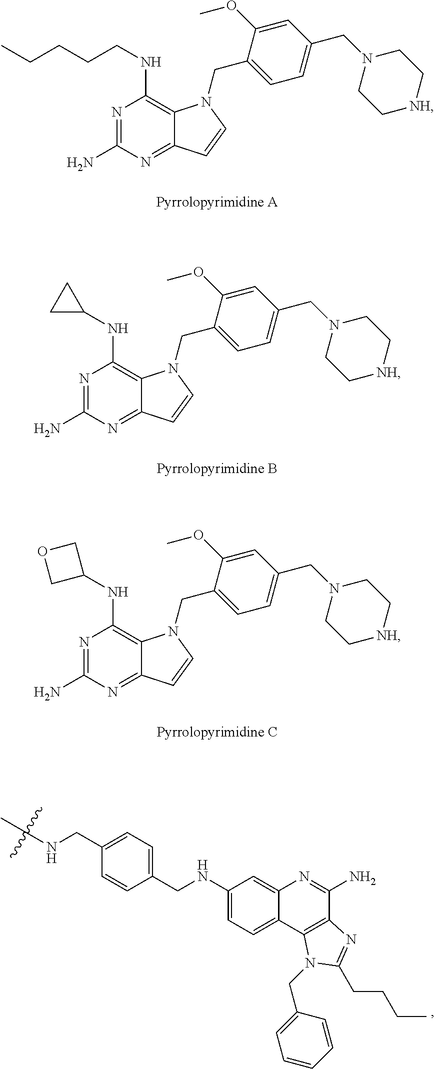 [SMILES: CCCCCNc1nc(N)nc2ccn(Cc3ccc(CN4CCNCC4)cc3OC)c12 ; COc1cc(CN2CCNCC2)ccc1Cn1ccc2nc(N)nc(NC3CC3)c21 ; COc1cc(CN2CCNCC2)ccc1Cn1ccc2nc(N)nc(NC3COC3)c21 ; *NCc1ccc(CNc2ccc3c(c2)nc(N)c2nc(CCCC)n(Cc4ccccc4)c23)cc1] 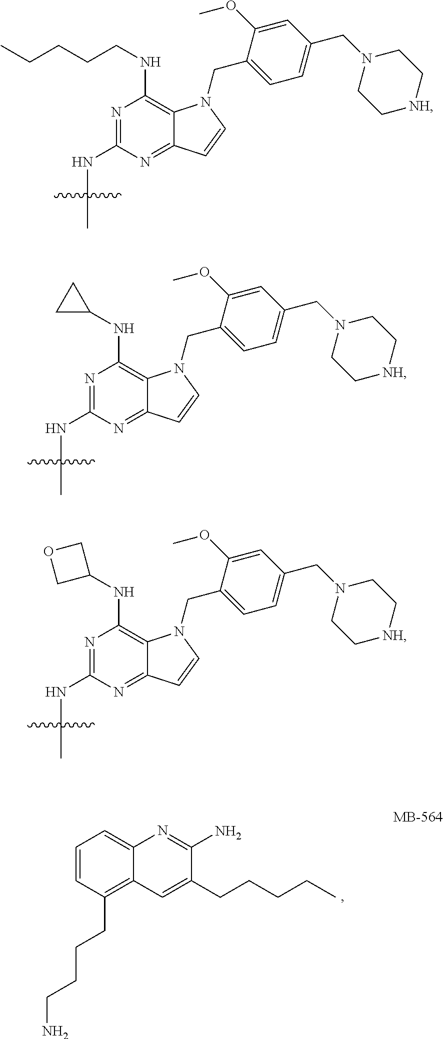 [SMILES: *Nc1nc(NCCCCC)c2c(ccn2Cc2ccc(CN3CCNCC3)cc2OC)n1 ; *Nc1nc(NC2CC2)c2c(ccn2Cc2ccc(CN3CCNCC3)cc2OC)n1 ; *Nc1nc(NC2COC2)c2c(ccn2Cc2ccc(CN3CCNCC3)cc2OC)n1 ; CCCCCc1cc2c(CCCCN)cccc2nc1N] 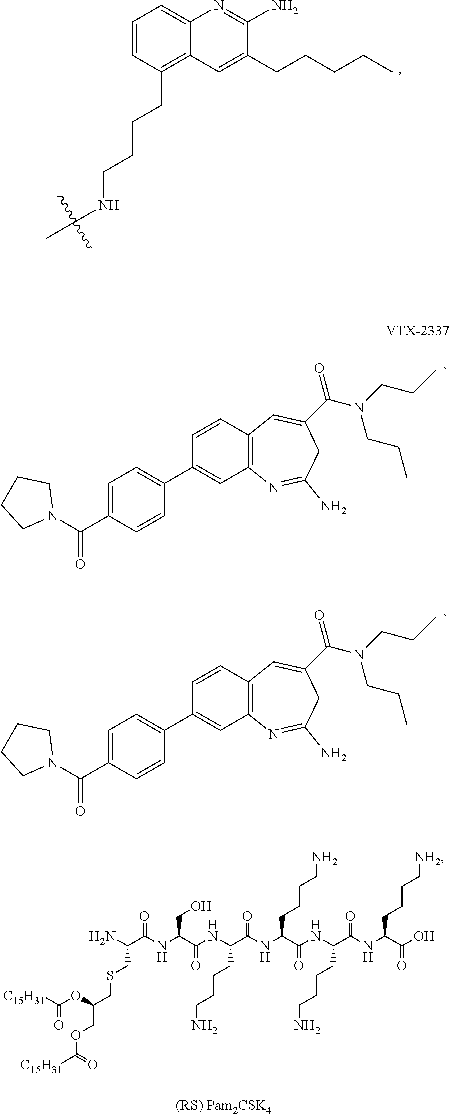 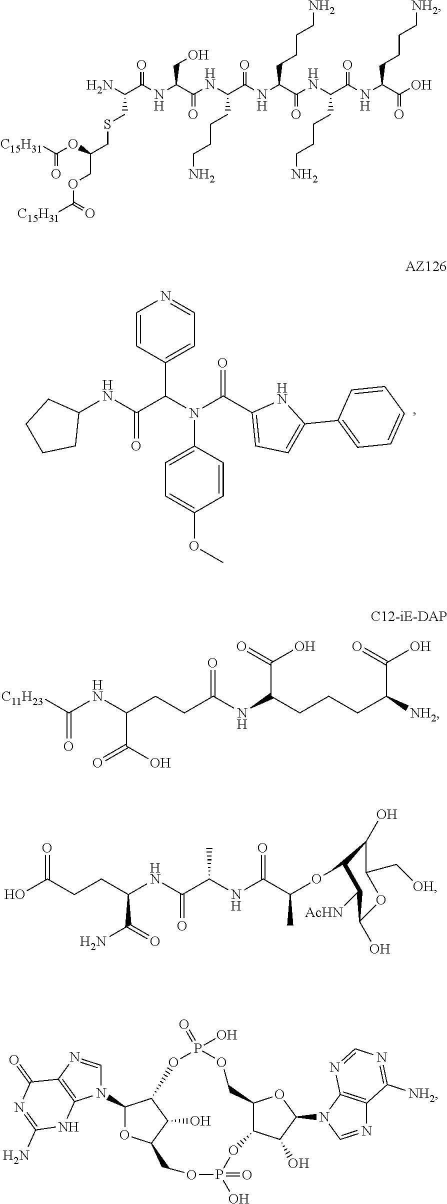
and D is

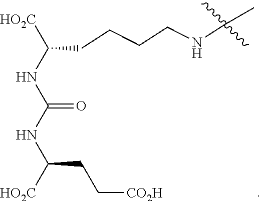 [SMILES: *NCCCC[C@H](NC(=O)N[C@@H](CCC(=O)O)C(=O)O)C(=O)O]

2. The compound of Formula I of claim 1, a diastereomer or enantiomer thereof, or a pharmaceutically acceptable salt of any of the foregoing, or a deuterated derivative or any of the forgoing, or a radioisotope of any of the forgoing, wherein:
A is selected from the group consisting of: ¹⁷⁷Lu-DOTA, ⁹⁰Y-DOTA, ²¹²Pb-DOTA, ²²⁵Ac-DOTA, ⁴⁷Sc-DOTA, ⁶⁷Cu-DOTA, and ¹³¹I-L-Tyrosine;
B is: 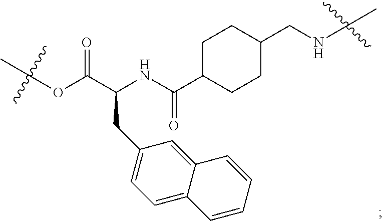 [SMILES: *NCC1CCC(C(=O)N[C@@H](Cc2ccc3ccccc3c2)C(=O)O*)CC1]
L₁ and L₂ are each independently selected from the group consisting up: (CH₂)ₙ, a dipeptide, a tripeptide, a disulfide, a hydrazone, a beta glucan, 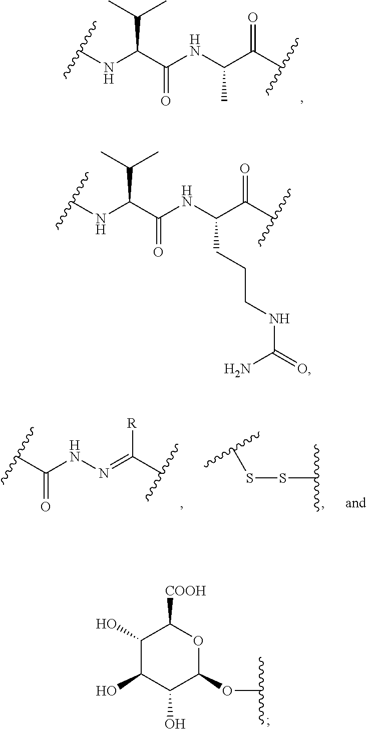
R is an aliphatic or unsaturated C1-C10, or an aromatic C6 or C10 group, wherein each R is optionally substituted with an amide, an ester, a C3-C10 cycloalkyl, an ether, or C1-C4 group;
each n is independently an integer 0-8;
C is selected from the group consisting of: 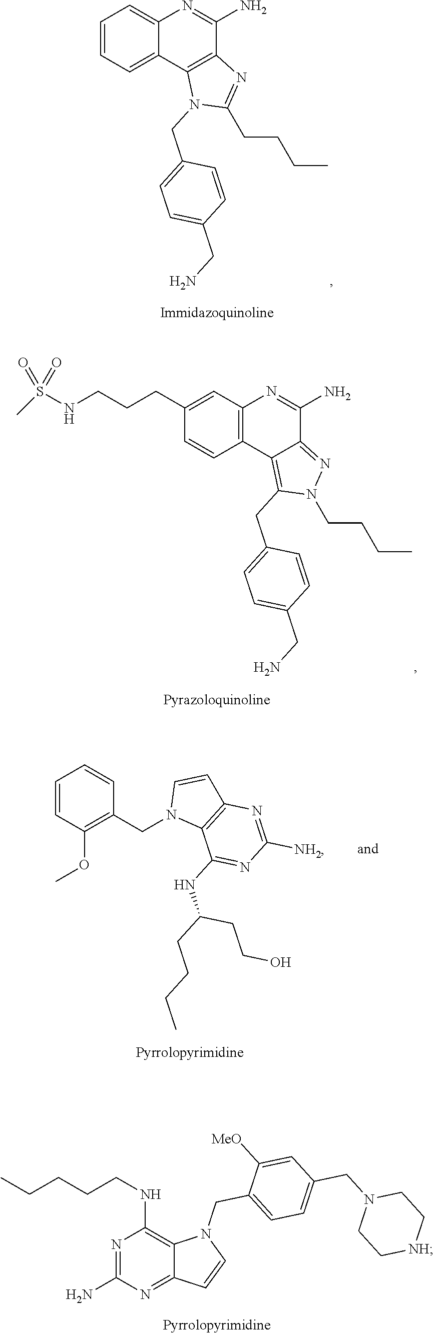
and
D is:

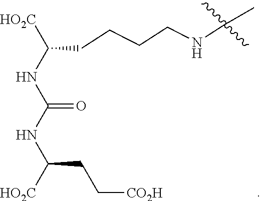 [SMILES: *NCCCC[C@H](NC(=O)N[C@@H](CCC(=O)O)C(=O)O)C(=O)O]

3. The compound of Formula I of claim 1, a diastereomer or enantiomer thereof, or a pharmaceutically acceptable salt of any of the foregoing, or a deuterated derivative or any of the forgoing, or a radioisotope of any of the forgoing, wherein
A is ¹⁷⁷Lu-DOTA;
B is: 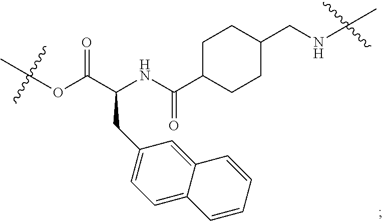 [SMILES: *NCC1CCC(C(=O)N[C@@H](Cc2ccc3ccccc3c2)C(=O)O*)CC1]
L₁ and L₂ are each independently selected from: (CH₂)ₙ, a dipeptide, a tripeptide, a disulfide, a hydrazone, a beta glucan, 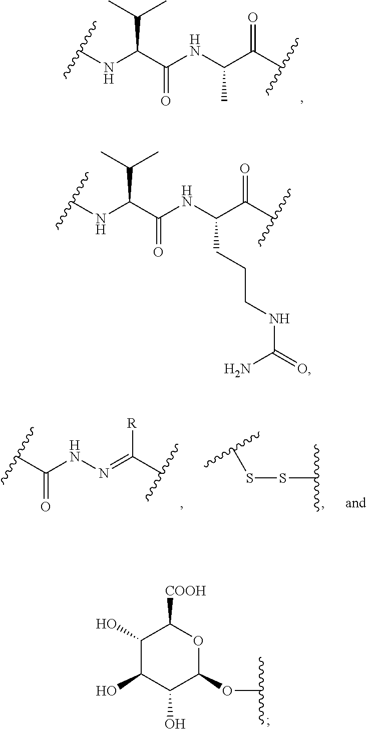
C is selected from the group consisting of: 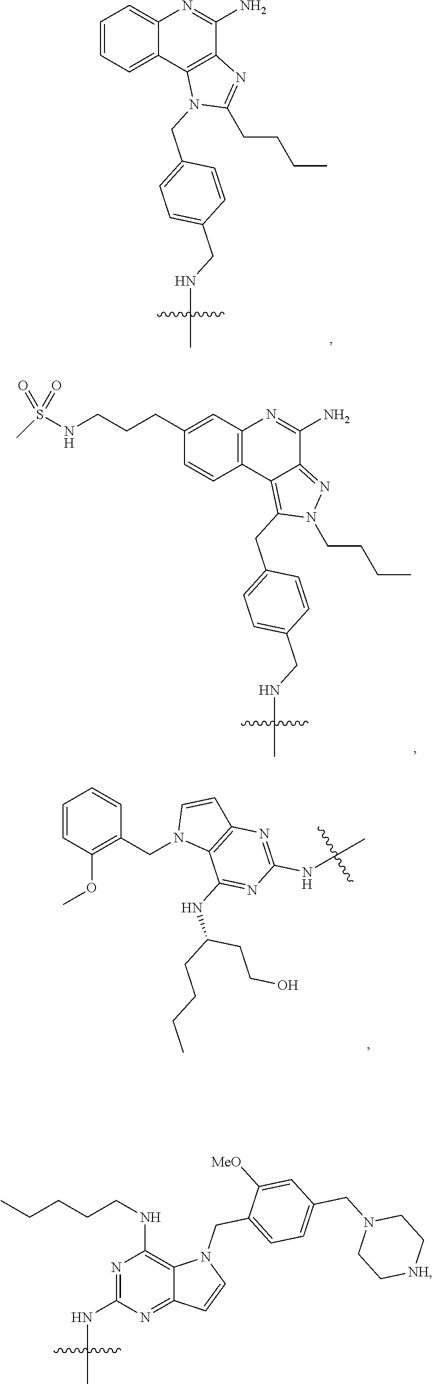 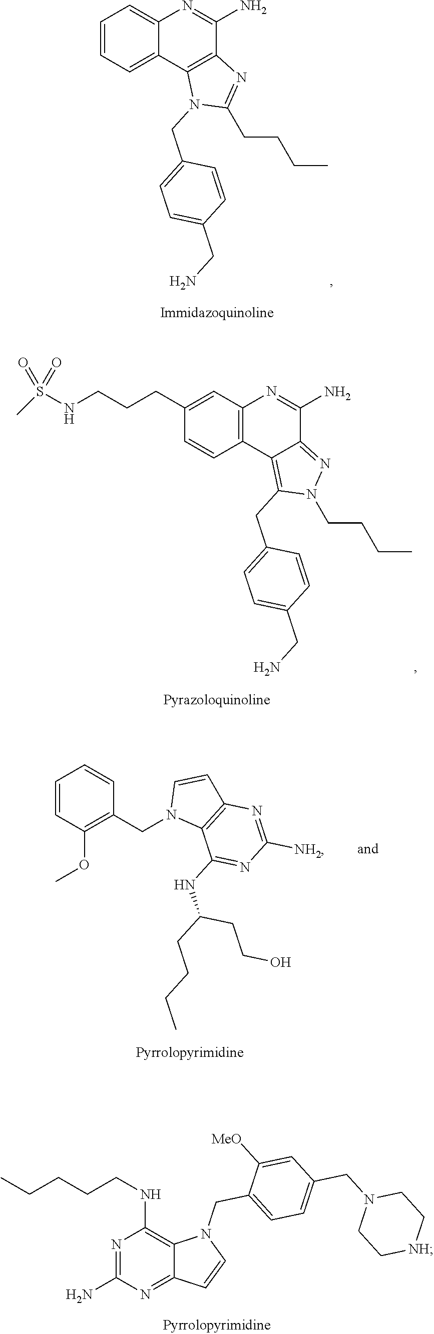
and
D is:

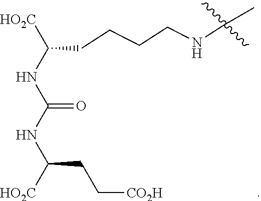 [SMILES: *NCCCC[C@H](NC(=O)N[C@@H](CCC(=O)O)C(=O)O)C(=O)O]

4. The compound of Formula 1 of claim 1, a diastereomer or an enantiomer of the compound, or a pharmaceutically acceptable salt of any of the foregoing, a deuterated derivative of any of the foregoing, or a radioisotope of any of the forgoing; wherein the compound of Formula I is selected from the group consisting of:

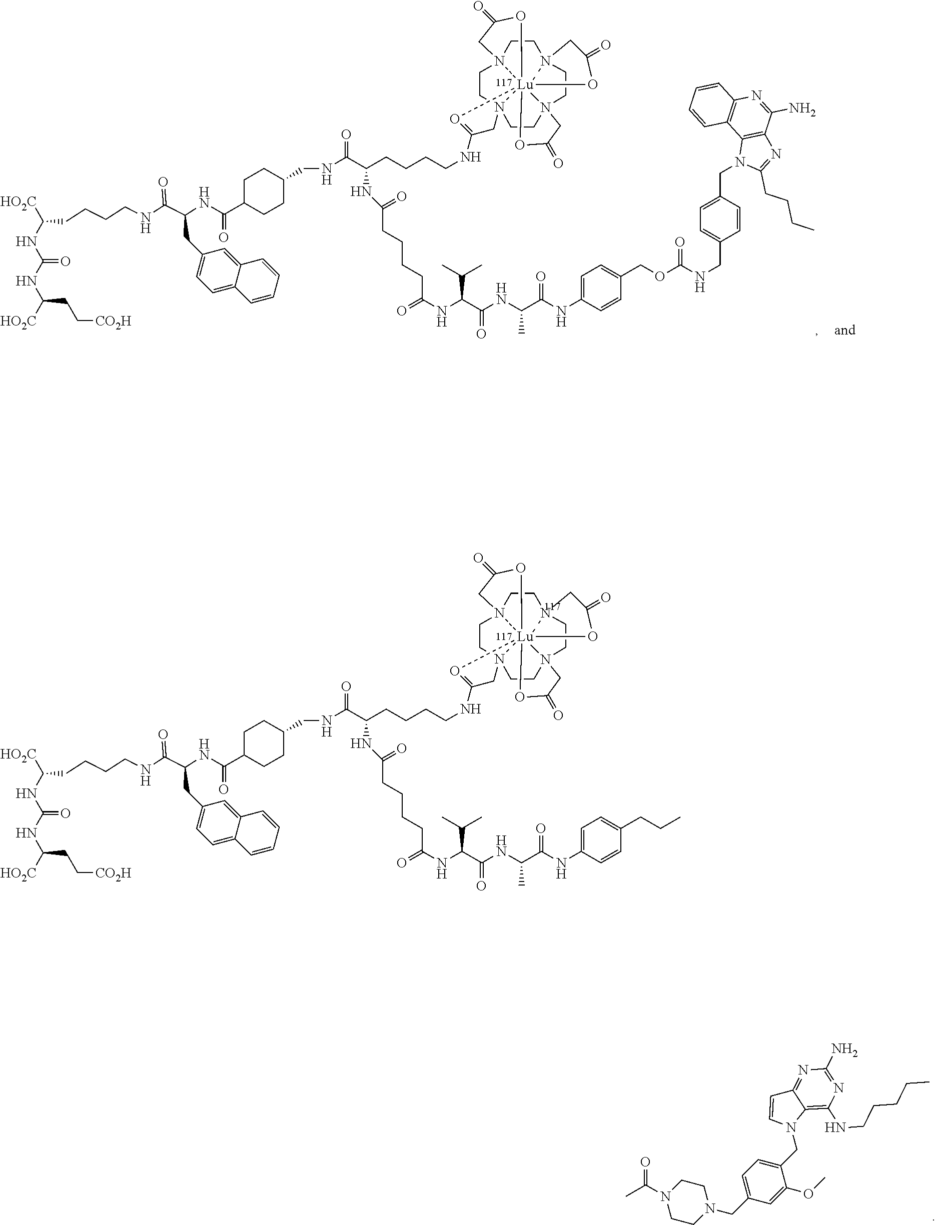

5. A pharmaceutical composition comprising, i) the compound of Formula I of claim 1; the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing; and ii) an excipient, diluent, or carrier.

6.-7. (canceled)

8. The pharmaceutical composition of claim 5, further comprising an additional active agent, or a pharmaceutically acceptable salt thereof.

9. The pharmaceutical composition of claim 5, that is in the form of a: tablet, powder, capsule, liquid, gel, emulsion, or suspension.

10. The pharmaceutical composition of claim 5, wherein the compound of Formula I, the diastereomer or the enantiomer of the compound, or a stereoisomeric mixture thereof of any of the foregoing, or the pharmaceutically acceptable salt of any of the foregoing, a deuterated derivative of any of the foregoing, or a radioisotope of any of the forgoing, is present in the pharmaceutical composition in an amount ranging from 0.001 mg to about 25,000 mg.

11. The pharmaceutical composition of claim 5, wherein the compound of Formula I is a radio-immunostimulant.

12.-17. (canceled)

18. A method of treating a cancer in a subject, the method comprising administering the pharmaceutical composition of claim 5 to the subject in a therapeutically effective amount, thereby treating the cancer.

19.-24. (canceled)

25. The method of claim 18, wherein the administering is: as needed, once per day, twice per day, three times per day, once per week, once per two weeks, once per three weeks, once per month, once every six months, once per year, or for life.

26. The method of claim 18, wherein the therapeutically effective amount ranges from about 0.001 mg to about 40,000 mg.

27. The method of claim 18, wherein the pharmaceutical composition comprises a radio-immunostimulant (RIMS).

28.-29. (canceled)

30. The method of claim 18, wherein the subject has a: prostate cancer, ovarian cancer, kidney cancer, colorectal cancer, NSCL cancer, castrate resistant prostate cancer.

31. The method of claim 30, wherein the cancer comprises the castrate resistant prostate cancer that has progressed to metastatic castrate resistant prostate cancer.

32.-33. (canceled)

34. A pharmaceutical composition comprising an immunostimulant, a cancer antigen targeting agent, a spacer molecule, a first linker, a second linker, and a cytotoxic agent, wherein:
the immunostimulant is a: TLR8 agonist, TLR7 agonist, TLR2 agonist, TLR4 agonist, NOD2 agonist, NOD1 agonist, or a STING agonist, wherein
(i) the immunostimulant is covalently bound to the first linker, and wherein the first linker is configured to release the immunostimulant when contacted with a cathepsin B mediated enzymatic cleavage;
the second linker is covalently bound to the cytotoxic agent, the spacer molecule, or a combination thereof; and
the cytotoxic agent comprises a radiotherapeutic, a small molecule, or a combination thereof, and wherein
the cytotoxic agent is covalently bound to the second linker, the spacer molecule, or a combination thereof, and wherein
the cancer antigen targeting molecule targets a PSMA cell, and is covalently bound to the spacer molecule; and
(ii) a pharmaceutically acceptable diluent, excipient, carrier.

35. The pharmaceutical composition of claim 34, wherein the pharmaceutical composition is a cancer vaccine in situ.

36. The pharmaceutical composition of claim 34, wherein the cytotoxic agent is selected from the group consisting of a: taxoid, vinca alkaloid, anthracycline, maytansinoid, tubulysin, auristatins, ¹⁷⁷Lu-H₃mpaten, ¹⁷⁷Lu-(picaga)-DUPA, ¹⁷⁷Lu-DOTA, ⁹⁰Y-DOTA, ²²⁵Ac-DOTA, ⁴⁷Sc-DOTA, ⁴⁷Sc-(picaga)-DUPA, ⁴⁷Sc—(H₃mpatcn) ⁶⁷Cu-DOTA, and ¹³¹I-L-Tyrosine.

37.-46. (canceled)

47. A composition according to the following formula: 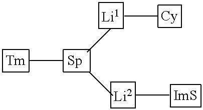
wherein:
Tm is a cancer antigen targeting molecule;
Sp is a spacer molecule coupled to the targeting molecule;
Li¹ is a first cleavable or non-cleavable linker coupled to the spacer molecule;
Li² is a second cleavable or non-cleavable linker coupled to the spacer molecule;
Cy is a cytotoxic small molecule coupled to the first cleavable or non-cleavable linker; and
ImS is a small molecule immunostimulant coupled to the second cleavable or non-cleavable linker.

48.-51. (canceled)

52. The compound of formula I of claim 1, comprising a compound according to the following formula:

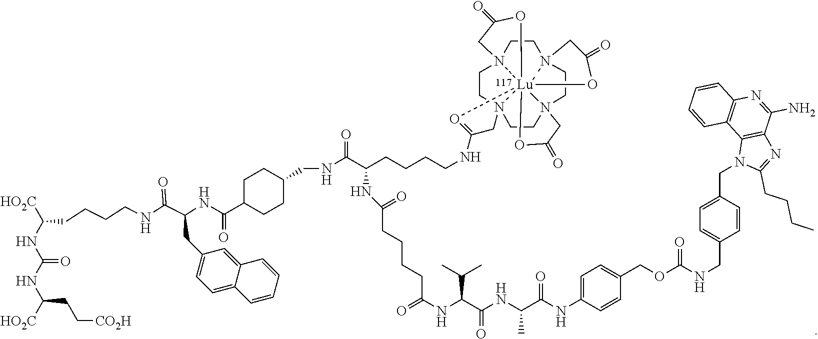

53.-56. (canceled)
